(19) United States
(12) Patent Application Publication
(10) Pub. No.: US 2021/0309702 A1
(43) Pub. Date: Oct. 7, 2021

(54) Programmable Designer Therapeutic Fusogenic Secreted Gectosome Vesicles For Macromolecule Delivery And Genome Modification

(71) Applicant: The Regents of the University of Colorado, a body corporate, Denver, CO (US)

(72) Inventors: Xuedong Liu, Denver, CO (US); Xiaojuan Zhang, Denver, CO (US); Zeyu Liu, Denver, CO (US); Quanbin Xu, Denver, CO (US)

(21) Appl. No.: 17/164,624

(22) Filed: Feb. 1, 2021

Related U.S. Application Data
(63) Continuation-in-part of application No. PCT/US2019/044686, filed on Aug. 1, 2019.
(60) Provisional application No. 62/713,289, filed on Aug. 1, 2018.

(51) Int. Cl. C07K 14/005; A61K 9/51; C12N 7/00; C12N 15/11; C12N 9/22; A61K 38/46; A61K 31/7088

(52) U.S. Cl. CPC C07K 14/005; A61K 9/5184; C12N 7/00; C12N 15/11; C12N 9/22; A61K 38/465; C12N 2800/80; C12N 2760/20022; C12N 2760/20033; C07K 2319/055; C07K 2319/035; C12N 2310/20; A61K 31/7088

(57) ABSTRACT

The invention includes systems, methods, and compositions for designing secreted fusogenic ectosome vesicles, or gectosomes, that selectively encapsulate specific target proteins, nucleic acids and/or other small molecules in a predetermined manner. These engineered gectosomes can be used to deliver desired cargos to receipt cells in vitro, ex vivo, or in vivo and may further reprogram target cellular phenotypes in a dose-dependent manner, as well as perform genome editing functions, among others.

### CROSS-REFERENCE TO RELATED APPLICATIONS

This application is a continuation-in-part application of PCT Application No. PCT/US2019/044686 having an international filing date of Aug. 1, 2019, which designated the United States, which PCT application claimed the benefit of U.S. Application Ser. No. 62/713,289, filed Aug. 1, 2018, both of which are incorporated by reference in their entirety.

### STATEMENT OF GOVERNMENT INTEREST

This invention was made with government support under grant numbers AR068254 and GM113141 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 1, 2021, is named “90245-00082-Sequence-Listing.txt” and is 6.95 Kbytes in size.

### TECHNICAL FIELD

The present inventive technology relates to systems, methods and compositions for the encapsulation and delivery of target molecules to recipient cells through secreted fusogenic vesicles.

### BACKGROUND

Effective delivery of genome editing enzymes, therapeutic RNAs, proteins, or small molecules into a cell transiently is vital to basic research and therapeutic development. For example, there has been tremendous progress in developing methods for gene modification and interfering with mRNA expression. Similarly, much effort has been put towards methods targeting inactivation of mRNA using antisense oligonucleotides, RNA interference, or the recently developed Cas13 for knocking down gene expression in the short term. All of these methods rely on delivering nucleic acid or protein-nucleic acid complexes to recipient cells. Those studying gene function in mammalian cells or animals have conventionally used virus- or lipid-mediated transfections to introduce DNA- or RNA-modifying machinery into the cell. Although these methods are universally employed and often effective, they have significant limitations in therapeutic applications. For example, virus-based delivery systems have been reported to increase patients' cancer risk and human immunity, in part due to persistent expression of Cas9. Lipid-based nanoparticles are limited by inefficient cargo release from endosomes, low targeting/fusion efficiency in vivo, poor cell or organ specificity, and relatively high toxicity. As a result, alternative methods for pharmacologically delivering cell function-modifying biologics are highly sought after.

One are of promising development has been in the use of extracellular vesicles (EVs) to deliver the encapsulated cargos, or target molecules including proteins, nucleic acids and small molecules to recipient cells in vitro and in vivo. EVs are heterogeneous nano-sized membrane vesicles constantly released by all cell types. Recent studies have identified EVs as an important mechanism for intercellular communication. Based on their size and biogenesis, EVs have been classified either as exosomes or microvesicles, also known as ectosomes. Microvesicles are formed and released by budding from the cell's plasma membrane and are generally 150-1,000 nm in diameter. Exosomes are smaller vesicles generally 40-150 nm in diameter and originate from endosomal compartments known as multivesicular bodies. The distinction between these two types of vesicles is complicated by the fact that both are highly heterogeneous with overlapping ranges of size and variable composition.

Until recently, mixtures of both types of vesicles were often investigated due to a lack of purification methods to separate them effectively. Adding to the complexity of their differentiation is the presence of other nanoparticles of similar size, such as apoptotic bodies, arrestin domain-containing protein 1-mediated microvesicles, and nucleosomes in the media or bodily fluids. Consequently, the functional capabilities of these two types of vesicles remain poorly understood. For example, EVs are known to encapsulate a variety of bioactive molecules, including proteins, nucleic acids, and lipids. Available evidence suggests that the proteome and cargo of microvesicles appear to be different from exosomes. This is not entirely unexpected, as exosomes are intraluminal vesicles formed by the inward budding of the endosomal membrane while swallowing up cytosolic proteins and RNAs during maturation of multivesicular endosomes. Release of exosome content occurs upon fusion of multivesicular endosomes with the cell membrane. In contrast, microvesicles are produced by an outward budding at the plasma membrane. However, it is still unclear how these vesicles selectively engulf cytosolic proteins or nucleic acids. Lack of control of the cargo encapsulated in either type of vesicle, coupled with their inherent heterogeneity, have hindered their functional analysis and the delineation of the basic rules governing cargo loading.

Notably, exosomes and microvesicles have emerged as a new way to deliver the encapsulated cargos, or target molecules including proteins, nucleic acids and small molecules to recipient cells in vitro and in vivo. Importantly, formation of ectosomes can be enhanced by overexpression of certain viral proteins such as vesicular stomatitis virus (VSV-G). Despite this, the use of ectosomes as a vehicle to deliver target molecules to eukaryotic cells is limited. For example, Mangeoti et al., (U.S. patent application Ser. No. 13/505,506), suggests using microvesicles as a delivery vehicle for proteins of interest in an in vitro system. However, such a system lacks the ability to program the microvesicle to provide the necessary specificity in the selection and transport of proteins necessary for precise diagnostic and therapeutic applications. Moreover, such unspecific application of microvesicles can result in unwanted cellular RNA contamination. For ectosomes to be an effective biologics delivery tool there has to be a way to control the type of cargos ectosomes can encapsulate without compromising its production and fusogenic activity.

To overcome the limitations outlined above, the present inventors present a general method for making programmable, highly fusogenic vesicles, which we call “Gectosomes” (such as, in one embodiment a VSV-G protein ectosomes), as vehicles for the dose-controlled delivery of bioactive macromolecules in vitro and in vivo. Incorporating mechanisms of vesicular stomatitis viral delivery and proficient fusogenic activity of vesicular stomatitis virus G protein (VSV-G), the present inventors developed an active cargo-loading strategy for Gectosomes using the split GFP system. Modeling and experimental studies show that active loading of Gectosomes via GFP complementation greatly increases the efficiency of cargo delivery to target cells and reduces non-specific encapsulation of cellular proteins.

The present inventors further demonstrated the versatility and broad applicability of this approach by the successful intracellular delivery of cytosolic and nuclear enzymes, resulting in the execution of DNA recombination, RNA interference, and gene editing in cultured cells and mice liver tissues in vivo. Since Gectosomes are genetically encoded, highly programmable, easy to prepare, and amenable to purification based on their cargo, this approach simplifies genome modification experiments and can be adapted to wide-ranging research and possible therapeutic applications.

### SUMMARY OF THE INVENTION

On aspect of the inventive technology generally includes systems, methods and compositions for an improved system for the encapsulation and delivery of target molecules to recipient cells through an EV, such as secreted fusogenic ectosome vesicles. In one preferred embodiment, the invention includes programmable or engineered secreted fusogenic ectosome vesicles, which may preferably be a gectosome (G protein ectosomes), configured to selectively encapsulate and deliver specific proteins, nucleic acids and small molecules to a recipient cell in a predetermined manner. Embodiments of the invention may also include a programmable or engineered gectosome vesicle that is configured to selectively encapsulate and deliver specific proteins, nucleic acids and small molecules, generally referred to as target molecules, to a recipient cell in a predetermined manner through the use of a split complement system, such as a split protein system and/or a protein-protein motifs. For example, in one preferred embodiment, a split protein system selected from the group consisting of: a split GFP system, a NanoBiT (Promega) split ubiquitin system, a split beta-gal system, a split luciferase system, a split mCherry system and the like.

Another aspect of the current inventive technology generally includes systems, methods and compositions for an improved system for the encapsulation and delivery of target genome-editing molecules to recipient cells through secreted gectosomes. In one preferred embodiment, the invention includes a programmable gectosome that is configured to selectively encapsulate and deliver specific genome-editing proteins to a recipient cell in a predetermined manner. Examples, may include, but not be limited to: Meganucleases (MGN), Zinc-Finger Nucleases (ZFN), Transcription Activator-Like Effector Nucleases (TALENs) and/or proteins related to the genome editing process known as Clustered Regularly Interspaced Short Palindromic Repeats (CRISPR), such as Cas9 or Cas13. In one specific embodiment, the invention includes systems and methods for pharmacologically delivering bioactive proteins, RNA-interfering machinery, and Cas(9 or 13)/sgRNA complexes, among other gene editing components in vitro and in vivo through the novel use of gectosomes. In one preferred embodiment, one or more gectosomes may be programed to effectuate the high-efficient intercellular transfer of their cargo to a variety of cell lines in vivo and in vitro, as well as select somatic tissue in live animals. In certain embodiments, the invention allows for the high-level purified of homogenous microvesicles with respective to their target cargo, thereby reducing undesirable bioactive contaminants.

Another aspect of the invention includes generalizable methods for active loading and purification of highly specific ectosome vesicles, or gectosomes, which are capable of effectively delivering genome-modifying tools to a variety of cells in vitro and in vivo. In one preferred embodiment, such gectosomes and are designed to co-encapsulate vesicular stomatitis virus G protein (VSV-G) with bioactive proteins, nucleic acid-modifying enzymes such as Cas9 or 13 via split protein complementation, such as a split GFP complement system. These fluorescent gectosomes can be purified away from contaminating extracellular vesicles and display higher specific activity due to the reduction of nonspecific incorporation of cellular proteins, overcoming a major obstacle of heterogeneity typically associated with extracellular vesicles. In other embodiments, gectosomes may be engineered that encapsulate various therapeutically relevant proteins, such as Cre, Ago2, SaCas9, and LwaCas13, that can execute designed modifications of endogenous genes in cell lines in vitro and somatic tissues in vivo, allowing for the targeted gectosome-mediated delivery of therapeutics for a wide range of human diseases.

Additional aspect may further include systems and methods for the generation of high-efficiency persistent gectosomes that may resist clearance by the immune system, for example through the expression of surface biomarkers that prevent immune system clearances. In one preferred embodiment, such a high-efficiency persistent gectosomes that may resist clearance by the macrophages through the overexpression and presentation of CD47 proteins on the surface of the gectosomes. In alternative aspects, in one embodiment the invention may include the overexpression of antibodies, or in a preferred embodiment a nanobody, such as anti-CD47 nanobody that may promote depletion of an EV or gectosome from circulation. In this embodiment, such EVs or gectosomes may be rapidly untaken by macrophage or dendritic cells and may more rapidly and/or effectively deliver a tumor antigen peptides to elicit an immune response.

One aspect of the inventive technology include systems, methods and compositions for making programmable, highly fusogenic gectosomes, which may be uses as vehicles for the dose-controlled delivery of specific pharmacological agents in vitro and in vivo. Another aspect of the inventive technology may include the novel use of vesicular stomatitis virus G protein (VSV-G) to stimulate production of fusogenic vesicles and mediate intercellular protein transfer. In certain preferred embodiment, a VSV-G promoted vesicle may encapsulate predetermined proteins and nucleic acids through a simple complementation process. In this preferred embodiment, the C-terminus of VSV-G protein may be coupled with a protein sequence element that drives loading of the desired interacting partners into VSV-G vesicles.

In another aspect, the invention may include systems, methods and compositions for making programmable, highly fusogenic gectosomes, which may be uses as vehicles for the dose-controlled delivery of specific pharmacological agents in vitro and in vivo, further incorporating one or more target proteins that may enhance cargo delivery to a target cell. In one preferred aspect, a terminus of VSV-G protein may be coupled with a protein sequence that increases delivery efficiency of the desired interacting partners into VSV-G vesicles. In one preferred aspect, a this increase cargo delivery efficiency may be accomplished through the expression of a peptide, or peptide fragment containing a p6^{Gag} peptide domain with a VSV-G protein. Co-expression of the p6^{Gag} peptide with a VSV-G protein may promote cargo escape from the endosome once a gectosomes enters a target cell.

In another aspect of the invention, a split GFP system may be used as a driver between VSV-G and the desired cargo proteins as such fluorescent gectosomes may be efficiently formed during shedding to the extracellular space. In another aspect, gectosomes with a desired cargo may be purified by fluorescence-activated cell sorting (FACS) to obtain nearly homogeneous particle populations. In additional aspects, the invention may include systems, methods and compositions for the cellular uptake of gectosomes and release of the cargo after cell contact with said gectosomes in a variety of cell lines and primary cells both in vivo and in vitro. In such a preferred embodiment, the invention may allow for homologous recombination, RNA interference, gene editing, and RNA ablation with designed gectosomes, for example in in vitro and in vivo systems. Additional aspects of the invention may include the clinical application of gectosomes for therapeutics by achieving in vivo editing of target genetic elements by transient delivery of genome editing molecules, such as Cas9/sgRNA among other target nucleases as well as other therapeutic compositions.

Yet, another aspect of the current inventive technology generally includes systems, methods and compositions for an improved system for the encapsulation and delivery of target ribonucleic acid or therapeutic RNA molecules to recipient cells through secreted fusogenic ectosome vesicles. In one preferred embodiment, the invention includes a programmable fusogenic ectosome vesicle, such as a gectosome, may be configured to selectively encapsulate and deliver specific RNAs and RNA-interference mediating proteins configured to elicit or enhance RNA-mediated interference in a recipient cell in a predetermined and/or dose-dependent manner.

In yet another aspect, the current inventive technology generally includes systems, methods and compositions for an improved system for the encapsulation and delivery of target polypeptides or therapeutic protein molecules, such as biologics, to recipient cells through secreted fusogenic ectosome vesicles. In one preferred embodiment, the invention includes a programmable fusogenic ectosome vesicle, such as a gectosome, that is configured to selectively encapsulate and deliver specific proteins, preferably therapeutic proteins to a recipient cell in a predetermined or dose dependent manner. In one preferred embodiment, such a protein, or protein fragment may be recognized as an antigen by the recipient cell and induce an immune response. As such, the current invention may include systems, methods and compositions for the vaccinating or prophylactically treating a recipient host.

Additional aspects of the current inventive technology generally includes systems, methods and compositions for an improved system for the encapsulation and delivery of target antibodies to recipient cells through secreted fusogenic ectosome vesicles. In one preferred embodiment, the invention includes a programmable fusogenic ectosome vesicle, such as a gectosome that is configured to selectively encapsulate and deliver specific antibodies to a recipient cell in a predetermined or dose dependent manner.

Additional aspects of the current inventive technology generally includes systems, methods and compositions for an improved system for the encapsulation and delivery of target small molecules or compounds to recipient cells through secreted fusogenic ectosome vesicles. In one preferred embodiment, the invention includes a programmable fusogenic ectosome vesicle that is configured to selectively encapsulate and deliver specific small molecules and/or compounds to a recipient cell in a predetermined or dose dependent manner.

One aspect of the invention may include systems, methods and compositions for the expression of a variety of viral glycoproteins that may be used to transfer programmable cargos between cells.

Another aspect of the inventive technology may include systems, methods and compositions for a programmable fusogenic ectosome vesicle, such as gectosome, that is configured to deliver one or more target molecules to a specific cell, and/or tissue and/or organism type. In a preferred embodiment, this may be accomplished through the expression of one or more viral glycoproteins that exhibit a distinct host and/or cell range.

Yet another aspect of the invention may generally include systems, methods and compositions for the formation and/or detection of ectosome formation through human Gag-like proteins. Another aspect of the current invention may include the use of programmable fusogenic ectosome vesicle that is configured to deliver one or more target molecules to treat a disease condition, preferably in humans.

Still further aspect of the invention may include systems, methods and compositions for the signal amplification of an immune system response in a subject. In one preferred embodiment, a donor cell may be transfected to heterologously express a fusion deficient fusogenic protein coupled with a first component of a split complement system as well as a second component of a split complement system fused with an antibody peptide or a tumor specific antigen peptide. The antibody peptide or a tumor specific antigen peptide may be anchored to a membrane capable of forming an EV by reconstituting said split complement system which may further encapsulate antibody peptide or a tumor specific antigen peptide in an EV. In this preferred embodiment, one or more epitopes of the antibody peptide or a tumor specific antigen peptide may be presented on the surface of the EV. As noted elsewhere, the reconstitute split complement system, or other tag may be detected and used to help isolate the subject EVs. A therapeutically effective amount of said isolated EVs may then be administered to a subject in need thereof wherein the antibody peptide or tumor specific antigen peptide presented on the surface of said isolated EVs may elicit an immune response in the subject.

Additional aspects of the inventive technology will be evident from the detailed description and figures presented below.

### BRIEF DESCRIPTION OF THE FIGURES

FIGS. 1A-K. Development of a Two-Component Fluorescent Gectosome for Intercellular Transfer of Specific Proteins. (A) The size distribution of VSV-G-sfGFP particles by flow cytometry using FACSAria Fusion Cell Sorter. Size reference beads were used as the standard. Dot plots are representative of three individual experiments. (B) Nanoparticle tracking analysis of the size distributions and concentrations of extracellular vesicles in the supernatants from sfGFP, VSV-G-sfGFP, or VSV-GGFP11/BlaM-Vpr-GFP1-10-transfected cells. Data are mean±SD for technical replicates (n=3) and are representative of 10 individual experiments. (C) Representative TEM and TEM immunogold images of VSV-G-sfGFP vesicles. Primary antibody: 8G5F11 VSV-G antibody; secondary antibody: goat antimouse IgG/M 6 nm. (D) Schematic illustration showing VSV-G Gectosome-mediated BlaM protein transduction and detection in the target cell. The schematic model is not drawn to the scale. (E and F) Flow cytometric analysis of CCF2-AM dye-loaded target HeLa cells (˜3 3 105) incubated with vesicles collected from the supernatants of the same number of producer 293T cells (˜106) transfected with the same amount of plasmids as shown. Representative dot plots and the quantification of BlaM-positive cells are reported (mean±SD, n=3). Data are representative of two individual experiments. (G) Schematic diagram of the Cre transduction experiment (not to scale). (H and I) Flow cytometric analysis of the color conversion of target cell 293ColorSwitch. 293ColorSwitch cells (˜1 3 105) were treated for 48 h with extracellular vesicles (total particle number, ˜4 3 109; GFP-positive particle number, ˜4 3 108) collected from supernatants of 293T cells transfected with plasmids as shown. Data are mean±SD (n=3), and dot plots are representative of three individual experiments. (J) Color conversion of target 293ColorSwitch cell (˜13105) with VSV-G/Cre Gectosomes (˜83108) or VSV-G-NJ/Cre Gectosomes (˜83108) in the presence of VSV-G antibody (8G5F11, 1:100) or control IgG; VSV-G-NJ is the G protein from vesicular stomatitis virus New Jersey strain. Data are mean±SD (n=3). (K) Performance of Gectosomes versus artificial liposomes in Cre delivery to 293ColorSwitch cells. The plot shows the percentage of cells switched upon exposure to the indicated liposomes or Gectosomes containing the indicated amount of Cre as determined by flow cytometry analysis. Data are mean±SD (n=3). Statistical significance for (F), (I), and (J) was assessed using Student's t test, ***p<0.001; n.s., not significant.

FIGS. 2A-I. Functional Separations of Gectosomes from Exosomes. (A) Schematic diagram of experimental design (not to scale). (B) Representative histograms of flow cytometric analysis of 293T cells transiently transfected with plasmids as shown. (C) Nanoparticle tracking analysis of EVs from culture supernatants of 293T cells transfected with the indicated plasmids. Data are mean±SD (n=3). (D) Flow cytometric analysis of 293ColorSwitch cells incubated with Cre Gectosomes, BlaM Gectosomes, or CD9/CD81-labeled exosomes. Representative dot plots and the quantification of switched cells are reported (mean±SD, n=3). (E) Western blot of Munc13-4, CD9, and GW130 in wild-type and Munc13-4 knockout 293T cells. (F) Flow cytometric analyses of the transfection efficiency of CD9-mCherry plasmid in wild-type and Munc13-4 knockout 293T cells. (G) Nanoparticle tracking analysis of the percentage of CD9 GFP11/Cre-GFP1-10-positive exosomes secreted from wild-type and Munc13-4 knockout 293T cells. (H) Nanoparticle tracking analyses of the concentrations of BlaM Gectosomes secreted from wild-type and Munc13-4 knockout 293T cells. (I) Flow cytometric analysis of CCF2 cleavage in HeLa cells transduced with BlaM Gectosomes from Munc13-4 knockout or wild type 293T cells. Data for (F)-(I) are mean±SD (n=3). Statistical significance was assessed using Student's t test. **p<0.001; n.s., not significant. See also FIGS. 10 and 11.

FIGS. 3A-F. Purification, Quantitation, and Mathematical Modeling of Gectosomes. (A) The flowchart of the Gectosome purification procedure. (B and C) Flow cytometric and western blotting analysis of Cre Gectosome fractions off the IZON qEVoriginal column. EVs pre-cleaned by 10,000 3 g centrifugation were loaded onto the IZON qEVoriginal column. The fractionations 1-8 were incubated with VSV-G antibody crosslinked magnetic beads. The beads were washed, and a portion of the beads was subjected to flow cytometric analysis (B) and western blotting analysis (C). UC denotes samples prepared by ultracentrifugation (100,000 3 g, 90 min). (D) The ratios of VSV-G-GFP11/Cre-GFP1-10 in factions 2 and 3 or UC were calculated based on band intensities in (C). (E) The percentages of VSV-G-GFP11, CD9, and GM130 in the fractions 2 and 3 versus the corresponding proteins in the UC sample were calculated based on relative band intensity in (C). (F) 3D mathematical modeling of the Cre Gectosome. The left panels show an outside view of a modeled prototypical Gectosome and its local zoom-in view. The right panel shows the middle intersection view of a Gectosome and its local zoom-in view. This 3D model is illustrated according to the space-filling of 5,620 VSVG-GFP11 molecules and 933 Cre-GFP1-10 molecules in a Cre Gectosome. The numbers of proteins of interest in this model are derived from the quantitative western blotting results in FIGS. 11B-11D and 3C. Data are mean±SD (n=3); statistical significance for (D) was assessed using Student's t test (**p<0.01; ***p<0.001). UC, ultracentrifugation. See also FIG. 11.

FIGS. 4A-F. Active Loading of Gectosomes via the Split GFP System Reduces the Passive Incorporation of Cellular Proteins. (A) Illustration of the experimental design showing the competitive encapsulation of cargo protein of interest into Gectosomes. Cre-GFP1-10 is the cargo protein of interest, and untagged BlaM is used as a proxy for measuring non-specific incorporation of proteins into Gectosomes. (B) The expression of VSV-G-GFP11, Cre-GFP1-10, and BlaM proteins in 293T cells lysates (left panel, ˜105 cells/lane) transfected with plasmids shown, and ultracentrifugation concentrated supernatants (right panel, ˜8 3 109 particles/lane). Balance refers to non-specific DNA that was included to ensure the same amount of total input DNA.

FIGS. 5A-G. Gectosomes Can Deliver Versatile Cargos into Target Cells and Program Gene Expression. (A) Confocal images of HeLa-Venus-Parkin-RFP-Smac cells transduced with Gectosomes carrying the indicated cargo proteins or nucleic acids. (B) Quantification of the percentage of cells with Parkin on mitochondria after 10-mM CCCP treatment for 2 h in (A). n>200 cells for each condition from 3 replicates. Data are mean±SD (n=3). (C) The RT-qPCR analysis of the efficiency of PINK1 knockdown in cells treated as indicated. The expression levels of PINK1 were normalized to that of GAPDH. Results are shown as the averages±standard error of the mean from two independent replicates (n=2). (D) Western blotting analysis of PINK1 protein in HeLa-Venus-Parkin cells treated as indicated. (E) Confocal images of HeLa-Venus-Parkin-RFP-Smac cells transduced with SaCas9/sgPINK1 Gectosomes or SaCas9/sgCtrl Gectosomes. (F) Quantitation of the percentage of cells showing Venus-Parkin accumulation on mitochondria with 10-mM CCCP for 2 h n>200 cells for each condition from three replicates. Data are mean±SD (n=3). (G) Western blotting analysis of PINK1 protein in HeLa-Venus-Parkin-RFP-Smac cells treated as shown. The relative amount of PINK1 in (D) and (G) was quantified by densitometry. Statistical significance for (B), (C), and (F) was assessed using the Student's t test. See also FIG. 12.

FIGS. 6A-F. CD47 Suppresses Gectosome Clearance by Macrophages. (A) Schematic illustration of the experimental procedure for evaluating the effect of CD47 on Gectosome clearance. (B) Western blotting analysis of cargo proteins in 293T cells and released Gectosomes. (C) Effect of CD47 or CD47 nanobody expression on the efficiency of Gectosome delivery of BlaM to HeLa cells. CCF2-loaded HeLa cells were incubated with VSV-G-GFP11/BlaM-GFP1-10 Gectosomes (with/without CD47-Myc-GFP11 or C47nb-Myc-GFP11 expression) and analyzed by flow cytometry. Data are mean±SD (n=3) and are representative of two individual experiments. (D) Measuring Gectosome depletion by RAW 264.7 cells. BlaM Gectosomes (with/without CD47 or C47 nanobody co-expression) (2 mL, ˜2 3 108/mL) were incubated with RAW 264.7 macrophage cells (˜106 cells/well in 6-well plate) for indicated time (0, 3, 6 h). After incubation, supernatants were retrieved by centrifugation at 1,000 rpm for 10 min to remove RAW264.7 cells. Subsequently, the retrieved supernatants (2 mL) were incubated with HeLa cells (˜33105 cells/well in 6-well plate) for 16 h, and then cells were loaded with CCF2-AM before they were harvested for flow cytometric analyses. The percentage of BlaM-positive cells was normalized to 0-h incubation. Data are mean±SD (n=3) and are representative of two individual experiments. Statistical significance was assessed using the Student's t test (**p<0.01; ***p<0.001). (E) Western blotting analysis of the proteins in Gectosomes collected from supernatants of 293T cells transfected with VSV-G-sfGFP with/without CD47-Myc-GFP11. (F) The levels of VSV-G-sfGFP Gectosomes in the mouse blood circulation 3 h after intravenous injection. Approximately 109 VSV-G-sfGFP Gectosomes resuspended in 150 mL PBS were injected per mouse. Fluorescent Gectosomes in plasma were collected by aldehyde sulfate beads as described in STAR Methods. These results are expressed as mean±SD (n=9) from three independent measurements for each mouse (n=3 mice). Statistical significance for (C) and (F) was assessed using the Student's t test; n.s., not significant. See also FIG. 13.

FIGS. 7A-E. PCSK9 Gene Editing in Mouse Livers through Systemic Gectosome Delivery of Gene-Editing Machinery. (A) Schematic diagram of in vivo mouse experiment. (B) Time course of serum PCSK9 levels in VSV-G/SaCas9/sgRosa26 group (n=3 mice), VSV-G/Sa-Cas9/sgPCSK9 group (n=3 mice), and VSV-G/SaCas9/sgPCSK9/CD47 group (n=4 mice). Each mouse received approximately 109 particles by tail vein injection four times at 48-h interval. (C) Western blotting analysis of PCSK9 in liver tissue of mice harvested from the control and treated groups. For the SaCas9/sgPCSK9/CD47 group, three out of four mice were randomly selected. Quantitation of PCKS9 levels normalized to the loading control (b-actin) is shown below the blot. (D) Time course of serum LDL cholesterol concentrations in mice injected with Gectosomes as in (B). (E) The body weights of mice were not significantly different between the treatment groups. Arrows show the times of tail vein injection. Data are mean±SEM; statistical significance for (C) was assessed using the Student's t test (*p<0.05). Two way ANOVA was used to determine the differences of all titers between groups in (B), (D), and (E) (*p<0.05; **p<0.01; ***p<0.001). See also FIG. 14.

FIGS. 8A-H. Development of a two-component fluorescent Gectosome for intercellular transfer of specific proteins. (A) Representative histograms of flow cytometric analysis of 293T cells transiently transfected with the plasmids as indicated. More than 10,000 events were scored for each condition. (B) Representative graphs of nanoparticle tracking analysis of extracellular vesicles in the culture supernatant from sfGFP, VSV-G-sfGFP, or VSV-G-GFP11 plus BlaM-Vpr-GFP1-10 transfected 293T cells. The profiles of supernatant in the clear scatter channel, and FITC channel are shown for each sample. Note that the scale of the y-axis is adjusted by NanoSight software for each sample for easy profile comparison. The size distributions (S) and particle concentrations (C) shown in the insets are the stock concentrations of each supernatant. Error bars indicate SD (n=3). (C) VSV-G-sfGFP Gectosomes bound with anti-VSV-G antibody-conjugated magnetic beads. The scale bar shows 400 μm. (D) Schematic illustration of one-component and two-component Gectosomes anchored by VSV-G fusion with sfGFP or split GFP. The schematic model is not drawn to the scale. (E) Confocal images of 293T cells transfected with plasmids as shown. (F) Western blot analysis of VSV-G or mutant VSV-G fusion proteins and BlaM fusion cargo proteins in Gectosomes from producer 293T cells. Each lane was loaded with a comparable number of cells or vesicles from a comparable number of cells. (G and H) Flow cytometric analysis of CCF2 dye loaded (or unloaded) target 293T cells (˜3×105) transduced with vesicles from the same number of producer 293T cells (˜106) transfected with the plasmids as shown. Data are mean±SD (n=3).

FIGS. 9A-J. Development of a two-component fluorescent Gectosome for intercellular transfer of specific proteins. (A) Representative histograms of flow cytometric analysis of 293T cells transiently transfected using VSV-G-GFP11/Cre-GFP1-10 or VSV-G-NJ-11/Cre-GFP1-10 plasmids. More than 10,000 events were scored for each condition. (B) Nanoparticle tracking analysis of VSV-G/Cre Gectosomes and the corresponding statistical result (n=10, mean±SD). (C) Confocal images of 293ColorSwitch cells after intake of VSV-G/Cre Gectosomes and VSV-G/BlaM (as control). (D and E) Flow cytometric analysis of target cell 293ColorSwitch cells after intake of VSV-G/Cre Gectosomes at the indicated time points (n=3, mean±SD). (F) The efficiency of Cre cargo delivery by two-component split GFP Gectosomes versus one-component VSV-G-Cre Gectosomes. 293ColorSwitch cells were exposed a similar number of EVs. The efficiency of switching was determined by flow cytometric analysis. Two independent biological replicates were performed with one-component Gectosomes. More than 10,000 events were scored for each condition. (G) Western blot analysis of VSV-G-Cre proteins present in secreted media supernatant (Sup) or 293T cell lysates transfected with VSV-G-Cre or mock. (H) Comparison of delivery efficiency of different vehicles with Cre cargo. The amounts of encapsulated Cre in Gectosomes and liposomes were estimated by Western Blotting using purified recombinant His-Flag-Cre protein as a protein standard. (I) Representative flow cytometric results of 293ColorSwitch cells transduced with Gectosomes or Liposomes loaded with the indicated amount of Cre. Data presents 10,000 events for each condition. (J) Curve fitting was generated by plotting the mean % ColorSwitch by the concentration of Cre loaded in Gectosomes. The green star denotes the conversion efficiency of liposome loaded with 510 nM of Cre is equivalent to the efficiency of Gectosomes with 0.8 nM of Cre.

FIGS. 10A-G. Functional separations of Gectosomes from exosomes. (A) CRISPR editing of Munc13-4 reduces exosome production without affecting Gectosome production. Representative histograms of flow cytometry of analysis of 293T and 293T Munc13-4 edited producer cells transfected with CD9-GFP11/Cre-GFP1-10. (B) NanoSight analysis of CD9 exosomes in the supernatant of transfected cells. (C) The depletion of CD9 exosomes does not affect Gectosome delivery. 293ColorSwitch cells (1×105) were incubated with a serial of dilution of Gectosomes that were treated with unconjugated magnetic beads or anti-CD9 magnetic beads. After the removal of magnetic beads, supernatants were incubated with 293ColorSwitch cells for 48 hr and analyzed by flow cytometry. (D) Quantitation of flow cytometry results in (C). Data are mean±SD (n=3). (E) Immunoblotting of CD9 in supernatants (˜1.5×108 particles) and on beads. (F) The effect of GW4869 treatment on Gectosome delivery of Cre. Immunoblotting of the pellets collected from supernatants of 293T cells treated with DMSO or 10 μM of GW4869 for 16 hr. Supernatants were cleared by 14K spin first followed by 100K ultracentrifugation spin to collect the pellet. The 100K pellets were used in the Western blot experiment. (G) 293T cells were treated with DMSO or 10 μM of GW4869 for 48 hr and supernatants were collected. Gectosomes were quantified by NanoSight. A serial dilution of Gectosomes was incubated with 293ColorSwitch cells (1×105). 48 hr after incubation, treated cells were analyzed by flow cytometry and the percentage of cells that were switched was quantified. Data are mean±SD (n=3).

FIGS. 11A-H. Purification, Quantitation, and Mathematical Modeling of Gectosomes. (A) Western blotting analysis of exosomal or ectosomal marker proteins in extracellular vesicles obtained from the indicated procedures. (B-D) Quantitative Western blotting analysis of the amounts of VSV-G-GFP11 and Cre-GFP1-10 in Cre Gectosomes. A known amount of recombinant GFP (rGFP) and His-Flag-Cre proteins were used as standards for quantitation. Gel image is representative of 3 individual experiments. (E) Schematic representation of the parameters and the scale used for the development of the 3D Cre Gectosome molecule model. (F) Log2Intensity distribution of identified proteins in immunoaffinity purified Gectosome samples. (G) MS analysis showing Peptide numbers, Intensity, and LFQ values of identified proteins in the indicated Gectosomes. Fold change for each parameter is shown below (VSV-G-GFP11/BlaM vs.VSV-G-GFP11/Cre-GFP1-10/BlaM). (H) Protein levels in Log2LFQ intensity values as determined by mass spectrometry of VSV-G/Cre-GFP1-10/BlaM Gectosomes (x-axis) compared with VSV-G/BlaM Gectosomes (y-axis). Red lines represent the gate of 4-fold change (FC).

FIGS. 12A-E. The versatility of loading Gectosomes with cargo proteins. (A) Representative histograms of flow cytometric analyses of 293T cells transiently transfected with the indicated expression vectors. (B) Confocal images of 293T cells transfected with the shown vectors. Scale bars, 10 (C) Western blotting analysis of proteins of interest in the indicated Gectosomes. (D) Representative histograms of flow cytometric analysis of HeLa-EGFP-PINK1 cells transduced with SaCas9/sgPINK1 Gectosomes. (E) Quantitation of the percentage of EGFP-PINK1 positive target cells treated with SaCas9/sgPINK1 and SaCas9/sgCtrl Gectosomes (Top panel); Western blotting showing EGFP-PINK1 protein levels in cells with the two treatments. Data are mean±SD (n=3); statistical significance for (E) was assessed using student's t test.

FIGS. 13A-C. CD47 suppresses Gectosome clearance by macrophages. (A) Effect of CD47 or CD47nanobody expression on the efficiency of Gectosome delivery of BlaM. The panel shows flow cytometric analyses of BlaM activity in HeLa cells loaded with the fluorescent CCF2-AM after incubation with VSV-G-GFP11/BlaM-GFP1-10 or VSV-G-GFP11/BlaM-GFP1-10/CD47-GFP11 or VSV-G-GFP11/BlaM-GFP1-10/CD47nb-GFP11 Gectosomes. (B) Depletion of VSV-G-GFP11/BlaM-GFP1-10 Gectosomes by RAW 264.7 macrophages after 16 h incubation as measured by fluorescence intensity using flow cytometry. Data are mean±SD (n=3) and are representative of 2 independent experiments. Statistical significance for (B and C) was assessed using the student's t-test (*p<0.05, **p<0.01). (C) Representative flow cytometric dot plots of VSV-G-sfGFP Gectosomes collected on Aldehyde Sulfate beads before or 3 h after intravenous injection. More than 10,000 events were scored for each condition.

FIGS. 14A-J. PCSK9 gene editing in mouse livers through systemic Gectosome delivery of gene-editing machinery. (A) Time course of serum PCSK9 levels in mice dosed by tail-vein injection of 109 particles of SaCas9/sgPCSK9 Gectosomes three times at 48 h interval. (n=5 for all titers and time points, error bars indicate SEM). (B) Western blotting analysis of PCSK9 protein in the liver tissue of three mice randomly chosen from the experimental groups in (A). Quantitation of PCKS9 levels normalized to the loading control ((3-actin) is shown below the blot. (C) Time course of serum LDL-cholesterol concentrations in mice as in (A). (n=5 for all titers and time points, error bars indicate SEM). (D) The body weights of mice were not significantly different between the treatment groups. Arrows indicate the time of injection. Data are mean±SEM; statistical significance for (C) was assessed using student's t test (*p<0.05). Two-way ANOVA was used to determine the differences of all titers between groups in (B, D, and E) (*p<0.05; **p<0.01; ***p<0.001). n.s., not significant. (E and F) Alignment of the PCSK9 amplified region from mouse livers edited by VSV-G/SaCas9/sgPCSK9 Gectosomes. The DNA sequencing results were shown in Supplemental Table 4. (G) Immunostaining of VSV-G and Cre in HeLa cells following VSV-G-GFP11/Cre-GFP1-10 Gectosomes exposure. Confocal imaging of HeLa cells at the indicated time points following Gectosomes incubation. Green signals represent split GFP complemented Gectosomes. The red channel shows VSV-G or Cre, respectively. DAPI stains nucleus. Orange boxes show the zoom-in section of the indicated areas. (H) Immunostaining of Cre and VSV-G of HeLa exposed to VSV-G-GFP11/Cre-GFP1-10 for 12 h. Red channel: Cre, white channel: VSV-G. The nucleus was stained with DAPI. (H) Immunostaining of split GFP, EEA1, and Lamp1 markers in HeLa cells following VSV-G-GFP11/Cre-GFP1-10 Gectosomes exposure. (I) Confocal imaging of HeLa cells at the indicated time points following Gectosomes incubation. Green signals represent split GFP complemented Gectosomes. Red signals show either EEA1 (top panel) or Lamp1 (Bottom panel). DAPI stains the nucleus. Orange boxes show the zoom-in section of the indicated areas. (J) Quantitation of co-localization of split GFP with EEA1 or Lamp-1. Data are expressed as Pearson's coefficient (r). The results show that the co-localization observed between split GFP and EEA1 is higher than that between split GFP and Lamp1. Data represent >37 cells from 3 replicates for each condition and are representative of 2 individual experiments. The statistical significance was assessed using student's t-test. **p<0.01.

FIGS. 15A-B. Visualization of Cre loaded gectosomes. (A) Confocal imaging of 293ColorSwitch cells following incubation with control and Cre gectosomes. Untreated control cells were positive for red fluorescence and no green cells were visible. Gectosome delivery of Cre should facilitate removal of DsRed cassette allowing eGFP expression. Because DsRed has a longer half-life, switched cells were positive for both DsRed and eGFP at the time of the measurement. (B) Quantitation of the efficiency of Cre-mediated color switch. Error bar, standard deviation.

FIGS. 16A-B. Gectosome-mediated protein transduction. (A) Gectosome-mediated protein transduction in immortalized or cancer cell lines. Cell lines were tested for BlaM activity after 16 h incubation with identical amounts of VSV-G-GFP-BlaM gectosomes. (B) Gectosomes can mediate protein transduction into MEF1, iPS, and primary cells isolated from mouse organs.

FIGS. 17A-G. Dose and kinetics of VSV-G gectosome delivery of bioactive proteins in cultured cells. (A) Efficiencies of BlaM-vpr protein transfer by gectosomes. The number of VSV-G-GFP11/BlaM-vpr-GFP1-10 fluorescent vesicles per mL was determined by NanoSight. A fixed number of HeLa cells (1×10⁶) were incubated with increasing number of gectosomes for 16 h prior to flow cytometric analysis of BlaM-positive cells. (B) Time course of BlaM cargo transfer. BlaM gectosomes were incubated with HeLa cells for indicated times prior to flow cytometric analysis of BlaM activity. (C) Intracellular degradation of BlaM protein in recipient cells. HeLa cells were incubated with a saturating dose of BlaM gectosomes for 16 h before media exchange, and the fraction of HeLa cells positive for BlaM at various hours following media exchange was determined by flow cytometry. (D) BlaM activity in recipient cells is not a result of new protein synthesis. HeLa cells transfected with BlaM expression plasmid or incubated with BlaM gectosomes were treated with cycloheximide (CHX) prior to flow cytometric analysis for BlaM activity. CHX was added to HeLa cells before exposure to gectosomes. (E) Schematic of Cre knockdown experiment to test whether gectosome-mediated protein transduction depends on its encoding mRNA or DNA. (F-G) 293ColorSwitch cells were programmed to be immune to incoming nucleic acid encoding Cre by transient expression of LwaCas13a with or without Cre sgRNA (unprogrammed) for 36 h. The unprogrammed control and programmed 293ColorSwitch cells were then exposed to Cre gectosomes or transfected with Cre-GFP1-10 expression vector. The efficiency of Cre transduction or Cre expression was measured by flow cytometric analysis of cells exposed to gectosomes or Cre overexpression. (F) Western blotting showing the expression LwaCas13-GFP1-10 and Cre-GFP1-10 proteins in 293ColorSwitch cells with GAPDH as the loading control. (G) percentage of switched cells.

FIGS. 18A-C. Purification of VSV-G gectosomes. (A) Schematic diagram of gectosome purification. (B) Immunoblotting analysis of VSV-G-sfGFP by two different methods of enrichment. The number of extracellular particles for each method as determined by NanoSight or flow cytometry loaded on the gel is indicated. The indicated amount of recombinant VSV-G protein (AlphaDiagnostics) was used as the standard for quantification of VSV-G-sfGFP in the particles. Bottom panel, Ponceau S staining of the nitrocellulose membrane prior to immunoblot. The 69 kDa band is probably bovine albumin protein from serum. (C) Quantitative immunoblotting analysis of cargo enrichment in gectosomes by two methods of purification as described in (B). The indicated amount of recombinant BlaM was loaded on the gel as the standard for quantification.

FIG. 19. Western blot analysis. Western blotting shows the VSV-G-GFP11, Cre-GFP1-10, and BlaM proteins harvested from HEK293T cells (the left panel) and the supernatant (the right panel, ultracentrifuged) from HeLa cells transfected using VSV-G-GFP11 with Cre-GFP1-10 and BlaM plasmids as in (A). The bottom panel shows the GAPDH loading control.

FIG. 20. Munc13D gene knockdown has no effect on the secretion and uptake of VSV-G-GFP-BlaM gectosomes. HEK293T cells were treated with CRISPR/Cas9/sgMunc13D and then transiently transfected using VSV-G-GFP11/BlaM-vpr-GFP1-10. After 48 h the supernatant was collected to infect HeLa cells, and then HeLa cells were loaded with the fluorescent CCF2 β-lactamase substrate to test BlaM activity. To investigate whether gectosome production is affected by Munc13-4 suppression, wild-type and Munc13-4 mutant cells were transfected with VSV-G-GFP11 and BlaM-vpr-GFP1-10. Gectosomes collected from these two cell lines were found to be equally potent in protein transfer upon incubation with HeLa cells; suggesting perturbation of Munc13-4 has minimal effect on gectosome secretion. Therefore, gectosomes and exosomes differ in protein transduction activity and requirements for their biogenesis. Munc13D gene knockdown has no effect on the secretion and uptake of VSV-G-GFP-BlaM gectosomes. HEK293T cells were treated with CRISPR/Cas9/sgMunc13D and then transiently transfected using VSV-G-GFP11/BlaM-vpr-GFP1-10. After 48 h the supernatant was collected to infect HeLa cells, and then HeLa cells were loaded with the fluorescent CCF2 13-lactamase substrate to test BlaM activity.

FIGS. 21A-B. Collection and purification of gectosomes. (A) RNAseq signal value of shRNA of PINK1 gene in VSV-G-GFP11/AGO2 gectosomes. HEK293T cells were transfected using VSV-G-GFP11/AGO2-GFP1-10 plasmids with control or shPINK1 plasmid. GFP-positive VSV-G-GFP11/AGO2 gectosomes were harvested and purified through flow cytometry using a BDAria Fusion cell sorter. RNA was extracted from sorted green particles and then submitted for RNAseq analysis. (B) RNAseq signal value of sgRNA of PINK1 gene in VSV-G-GFP-SaCas9 gectosomes. HEK293T cells were transfected using VSV-G-GFP11/SaCas9-GFP1-10 plasmids with an sgPINK1 or mock plasmid. GFP-positive VSV-G-GFP11/SaCas9 gectosomes were harvested and purified through flow cytometry using a BDAria Fusion cell sorter. RNA was extracted from sorted green particles and then submitted for RNAseq analysis.

FIGS. 22A-B. VSV-G like viral glycoproteins and human endogenous Gag-like can be repurposed for ectosome mediated intercellular transfer of biologics and genome editing. (A) Nanosight analyses of ectosomes produced by 293T cells transfected with GFP11 tagged viral glycoproteins and human Gag-like proteins co-expressed with BlaM-Vpr-GFP1-10. (B) Cell type specificity of CNV-G ectosomes in transferring of proteins.

FIGS. 23A-B. (A) Schematic illustration of vectors for production of Gectosomes, including exemplary use of p6^{Gag} peptide motif from human immunodeficiency virus type 1 (HIV-1) Gag protein, to enhance gectosome protein (Cre) cargo delivery efficiency, such constructs comprising: VSV-G-p6^{Gag}-GFP11; VSV-G-GFP11; and Cre-GFP1-10. (B) Comparison of the efficiency of Cre-GFP1-10 delivery to 293T or HeLa ColorSwitch cells by VSV-G-GFP11 or VSV-G-p6^{Gag}-GFP11. Equal number (1.25×10⁹) of VSV-G-GFP11/Cre-GFP1-10 or VSV-G-p6^{Gag}-GFP11 Gectosome were incubated with ˜1×10⁵ 293 T ColorSwitch or HeLa ColorSwitch cells for 48 hr before they were harvested for flow cytometry analysis. Percentage of switched cells is indicated in the plots. Cargo delivery of Cre-GFP1-10 is at least 20% more efficient with p6^{Gag} variant of VSV-G.

FIG. 24. (A) Schematic diagram of a two-hybrid gectosome system for mRNA loading and intercellular mRNA transfer. (B) RT-PCR analysis of gectosomes purified from 293T cells transfected with indicated combination of expression vectors. MCP: MS2-coat protein; MS2: MS2 coat protein binding sequence. CFP: cyan fluorescence protein. M:1 kb DNA ladder.

FIG. 25. Knock down expression of HBx in Hep3B cells using gectosomes encapsulated with indicated cargoes in (A). (B) qPCR quantitation of the knockdown effects. (C) Immuno-staining of HBx in treated cells.

FIG. 26A-B. (A) Schematic illustration of vectors for production of Gectosomes. (B) Delivering Cre mRNA with gectosomes. L7Ae is a RNA binding protein that interacts with mRNA through CD Box. Equal number (1.25×10⁹) of VSV-G-p6Gag-GFP11 with L7Ae-GFP1-10, Cre-GFP1-10 or L7Ae-GFP1-10 plus Cre-BoxCD mRNA. Gectosome were incubated with ˜1×10⁵ 293 T ColorSwitch cells for 48 hr before they were harvested for flow cytometry analysis. Percentage of switched cells is indicated in the plots.

### DETAILED DESCRIPTION OF THE INVENTION

Generally, the inventive technology includes systems, methods and compositions for making a specific type of EVs called Gectosomes, which facilitate cargo loading and their endosomal escape simultaneously. In one embodiment, Gectosomes contain two major components: an engineered VSV-G and the cargo of interest tethered to one another via split Split-Fluorescent proteins (SFPs) including Split-Green Fluorescent Proteins (GFP). Complementation of split-GFP enables more efficient loading of the specific cargo and purification of desired fluorescent Gectosomes. As detailed below, such engineered Gectosomes may be configured to deliver proteins or protein/RNA complexes designed to modify genotypes in mammalian cells in vitro and in vivo.

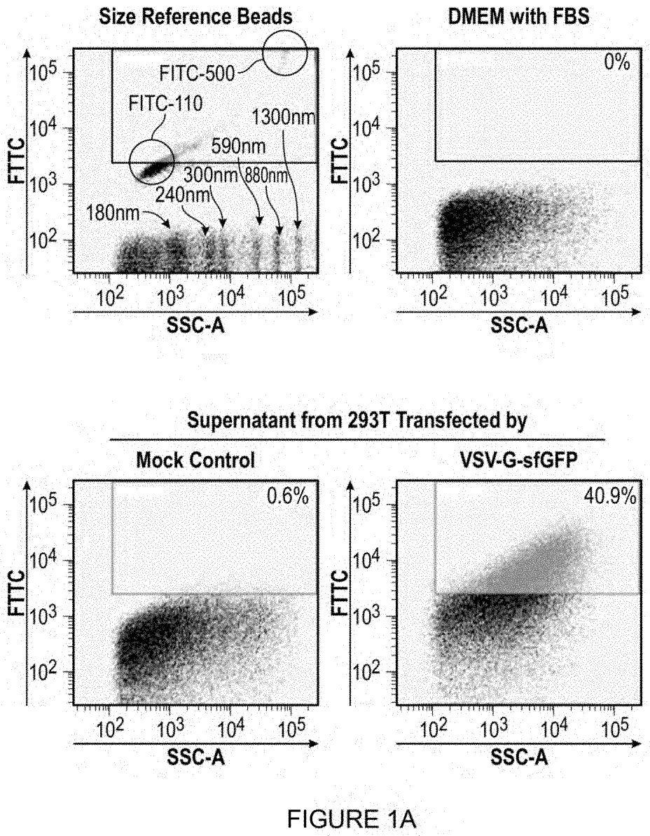

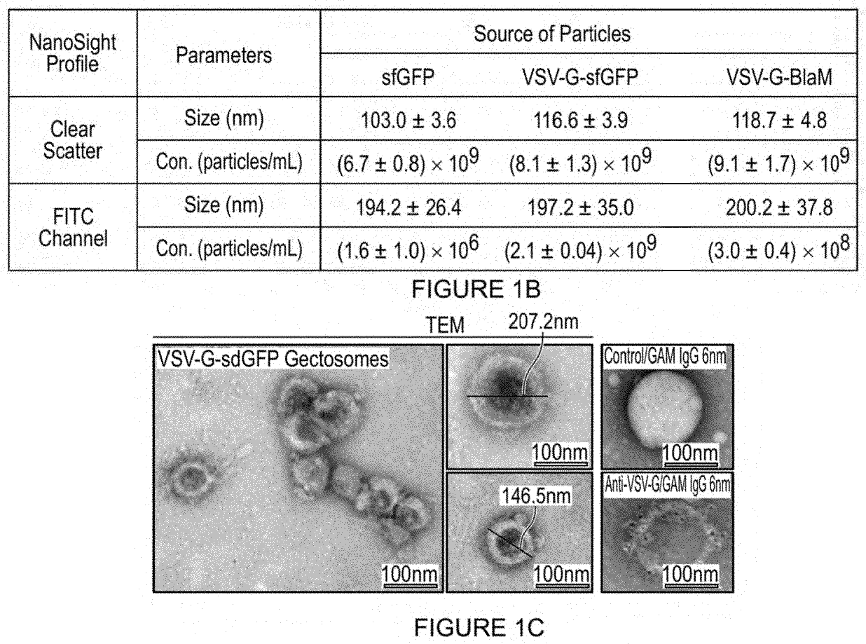

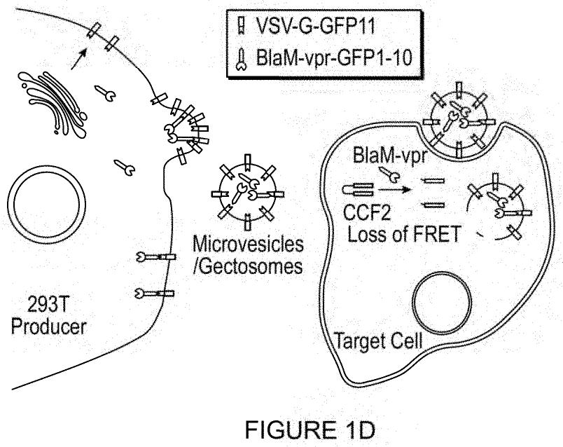

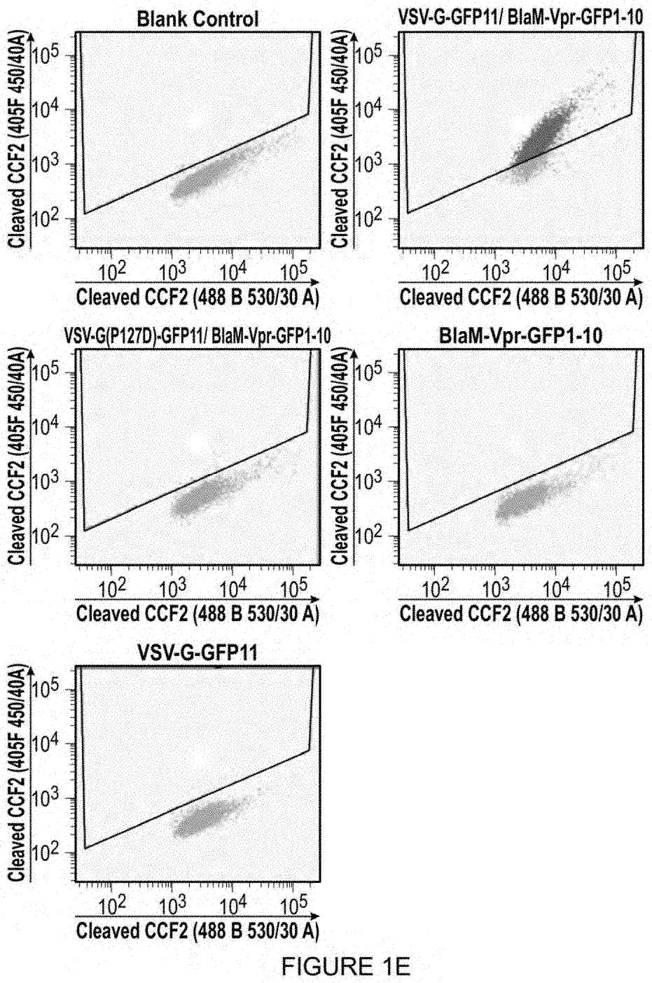

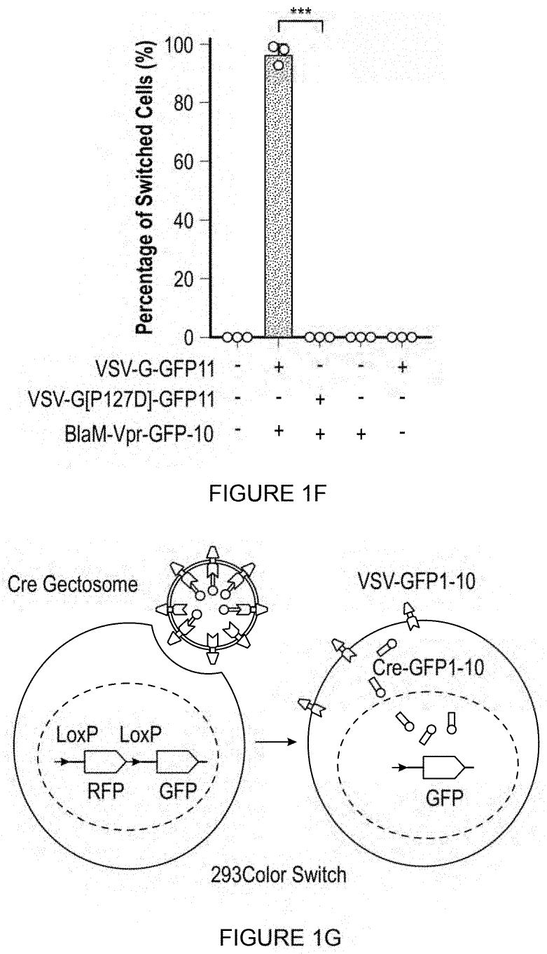

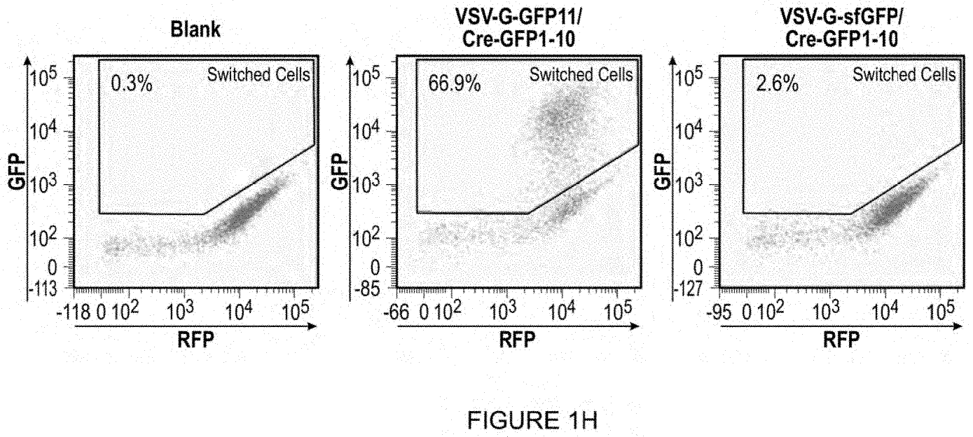

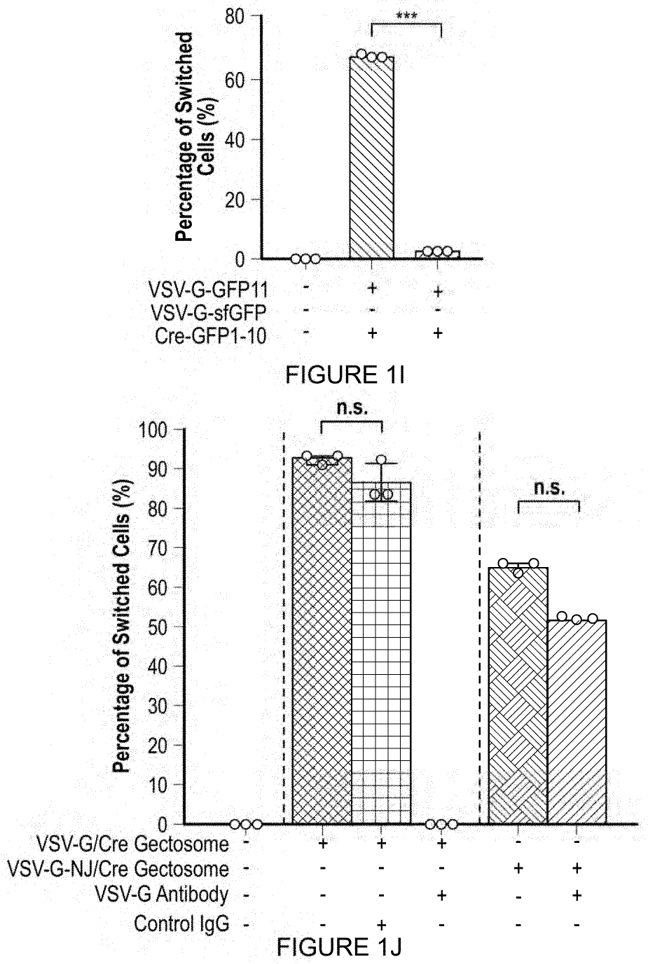

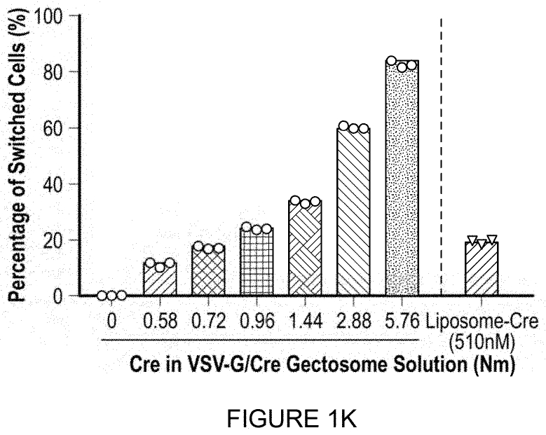

In one preferred aspect, the inventive technology includes systems, methods and compositions for the in vitro and/or in vivo generation of engineered or programmable fusogenic secreted vesicles that may be configured to be loaded with one or more specific target molecules. As generally shown in the FIG. 1D, in one embodiment, a donor cell may be engineered to generate fusogenic secreted vesicles having a targeting moiety expressed on the surface of the vesicles. This targeting moiety may include a protein, or protein fragment that may bind to a moiety present on a target or recipient cell. As described below, in one preferred embodiment an engineered fusogenic secreted vesicles may include a VSV-G protein that has been engineered to further include an interacting moiety that can be recognized by encapsulated proteins directly or indirectly to form interacting complexes. This interacting moiety may further be configured to be recognized by encapsulated proteins that may further bind proteins, protein fragments, nucleic acids, and/or small molecules as generally described herein.

Again, referring generally to FIG. 1D, in one embodiment the invention may further include methods for the generation and/or loading of engineered fusogenic secreted vesicles, such as gectosomes, with one or more proteins, nucleic acids, and/or small molecules as generally described herein. In one preferred embodiment, an engineered fusogenic secreted vesicle may be loaded with a target cargo through electroporation, liposomal transfection or fusion with other types of vesicles among other mechanisms known in the art. The invention may further include systems, methods and compositions for the generation of engineered fusogenic secreted vesicles, generally referred to as gectosomes, having VSV-G, or related viral G proteins and/or other microvesicle producing proteins that contain an interacting moiety that can be recognized by encapsulated proteins that bind proteins/peptides, nucleic acids or small molecules as generally outlined herein.

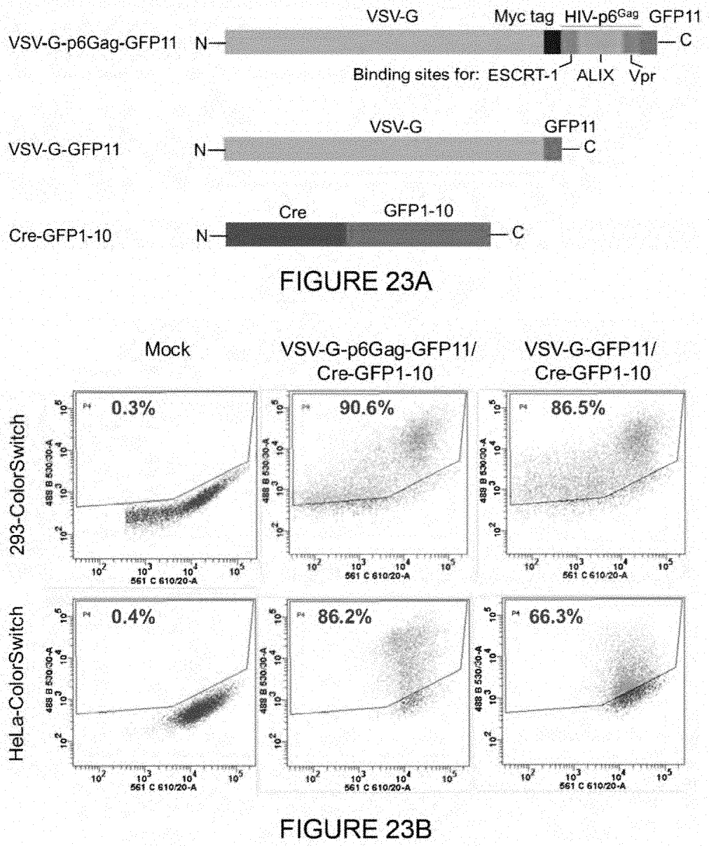

Generally referring to FIG. 23, in a preferred embodiment the invention may include systems, methods and compositions for making programmable, highly fusogenic gectosomes, which may be uses as vehicles for the dose-controlled delivery of specific pharmacological agents in vitro and in vivo, further incorporating one or more target proteins that may enhance cargo delivery to a target cell. In one preferred aspect, a terminus of VSV-G protein (SEQ ID NO. 1) may be coupled with a protein sequence element that increases delivery efficiency of the desired interacting partners into VSV-G vesicles. In one preferred aspect, a this increase cargo delivery efficiency may be accomplished through the expression of a peptide, or peptide fragment containing a p6^{Gag} peptide domain with a VSV-G protein. Co-expression of the p6^{Gag} peptide (SEQ ID NO. 2) with a VSV-G protein may promote cargo escape from the endosome once a gectosomes enters a target cell. In one embodiment, expression of VSV-G protein and a p6^{Gag} peptide may be from the same expression cassette forming a fusion protein. In this embodiment, the VSV-G protein and a p6^{Gag} peptide may be coupled with a linker or other spacer element, or a tag, such as a myc-tag. The p6^{Gag} peptide may include a domain directed to the Endosomal Sorting Complex Required for Transport (ESCRT), with binding sited for ESCRT-1, ALIX and Vpr.

The invention may further include systems, methods and compositions for the generation of engineered fusogenic secreted vesicles having human gag-like endogenous proteins and an interacting moiety that can be recognized by encapsulated proteins that bind proteins/peptides, nucleic acids or small molecules as generally outlined herein. In a preferred embodiment, an engineered fusogenic secreted vesicle may include human gag-like endogenous proteins and an interacting moiety that can perform perturbation of gene functions such as Cas9, dCas9, SaCas9, dSaCas9, LwaCas13, Cas13, C2c1, C2C3, C2c2, Cfp1, CasX, base editor, CRISPRi, CRISPRa, CRISPRX, CRISPR-STOP and base editors as generally described herein.

In one embodiment the invention may include the loading of a recombinase enzyme, such as a Cre recombinase, into an engineered fusogenic secreted vesicle, such as a gectosome. This Cre recombinase may further be transported via VSV-G mediated transfer from donor cells to target cells resulting in a permanent change coding genome in the recipient cell. As described below, HEK293 CRE reporting cell line expresses a reporter gene containing DsRed with a stop codon flanked by two LoxP sites upstream of GFP. Without CRE, CMV promoter drives the DsRed high expression to the stop codon and cells display strong red fluorescence. The downstream GFP ORF was not expressed because of the stop codon after the DsRed. Upon introduction of Cre via VSV-G ectosomes, the CRE excises/deletes the DNA fragment between two loxP sites, which remove the stop codon, resulting in strong green fluorescence as detected by flowcytometry. The examples provided below further demonstrate the conversion efficiencies of VSV-G, VSV-G-GFP11 with Cre-GFP1-10 in this embodiment.

As noted above, in one preferred embodiment an engineered fusogenic secreted vesicles may include a VSV-G that has been engineered to further include an interacting moiety that can be recognized by encapsulated proteins directly or indirectly to form interacting complexes. In one preferred embodiment, one or more target molecules may be selected through direct and/or indirect interaction with VSV-G, or other fusogenic proteins, such as viral glycoproteins. For example, in certain preferred embodiments, VSV-G like proteins in Ebola, Rabbies, Heptatis C, Lymphocytic choriomeningitis (LCMV), Autographa californica nuclear polyhedrosis virus (AcMNPV) and Chandpura (CNV) may be utilized to produce programmable ectosomes that can be used for transferring proteins, RNAi and/or genome editing agents as generally described herein. As described herein, such fusogenic proteins may not only promote production of programmable ectosomes but may also exhibit a distinct host and/or cell range. For example, in one embodiment, a viral G protein, such as CNV-G may be used to generate programmable ectosomes. Such CNV-G derived programmable ectosomes may predominantly target neuronal and lymphocytes. As such, the inventive technology allows for the generation of cell, tissue, and/or organisms' specific programmable secreted fusogenic ectosome vesicles.

In another embodiment, the inventive technology may further include the generation of secreted fusogenic vesicles, such as gectosomes, that may further be introduced to proteins, nucleic acids or small molecules of the type generally described herein. In one embodiment, secreted fusogenic vesicles that may be electroporated or transfected with proteins, nucleic acids or small molecules as generally identified herein. In one embodiment, self-complementing split fluorescent proteins (SFPs) may be used to generate two-component fluorescent gectosomes with recombinant VSV-G variants. Such VSV-G ectosomes may be configured to mediate the transfer of VSV-G interacting proteins from a donor cell to a target cell. SFPs are a protein complex composed of two or more protein fragments that individually are not fluorescent, but, when formed into a complex, result in a functional (that is, fluorescing) fluorescent molecule. Complementary sets of such fragments are also known as a SFP system, and typically include a SFP detector (comprising 9-10 strands of an 11 β-barrel fluorescent protein) and one or two SFP tags (comprising the remaining strands of the fluorescent protein). The SFP detector complements with the heterologous SFP tag (or tags) to form a functional (that is, fluorescing) fluorescent protein. Thus, an SFP tag and the complementary SFP detector are two complementing fragments of an SFP. In certain embodiment, a split GFP system may include a detector of GFP1-10 and a GFP11 tags (See FIG. 23). Polypeptides comprising Split-GFP fragments are known to the skilled artisan and further described herein. See, e.g., U.S. Pat. App. Pub. No. 2005/0221343 and Int. Pat. App. Pub. No. WO/2005/074436, and Cabantous et al., Nat. Biotechnol., 23:102-107, 2005; Cabantous and Waldo, Nat. Methods, 3:845-854, 2006. Other variations are also available; see, e.g., U.S. Pat. App. Pub. No. 2005/0221343. The polypeptides comprising complementing Split-GFP fragments disclosed herein will form a functional GFP molecule when complemented.

Construction of a test protein fused to a SFP tag or SFP detector is typically accomplished via cloning of the nucleic acid encoding the test protein into a nucleic acid construct encoding the SFP tag or SFP detector. SFPs, SFP systems, a number of specifically engineered tag and detector fragments of a SFP, such as split GFP systems, as well as DNA constructs and vectors use thereof are disclosed herein and known to the skilled artisan. See, e.g., U.S. Pat. App. Pub. No. 2005/0221343; Int. Pat. App. Pub. No. WO/2005/074436; Cabantous et al., Nat. Biotechnol., 23:102-107, 2005; Cabantous and Waldo, Nat. Methods, 3:845-854, 2006.) Typically, the SFPs include two SFP fragments, such as a SFP tag (typically corresponding to GFP11) and a SFP detector (typically corresponding to GFP1-10). Other SFPs are disclosed herein.

In certain embodiments, several VSV-G variants may be generated. Such VSV-G variants may contain a short peptide tag derived from a split protein system which enables VSV-G to form stable complex with any protein(s) that is fused to its complementary fragment. For example, in one embodiment a VSV-G was fused to a 16 amino acid peptide tag (GFP11). This fusion generates fluorescence when co-expressed with its complementary fragment, GFP1-10. In one preferred embodiment, an amino acid peptide tag GFP1-10 may be fused with a target molecule, such as a protein that can modify gene expression or have some other phenotypic or therapeutic effect on the target cell. In this embodiment, the GFP1-10-fusion may be co-expressed with, for example, VSV-G-GFP11, resulting in the transfer functionality from donor cells to recipient cells with high fidelity.

As noted above, the invention may include the use of secreted fusogenic vesicles, such as gectosomes, to transfer new and/or enhanced phenotypic, enzymatic, or even metabolic changes to a recipient cell. For example, in one embodiment, secreted fusogenic vesicles that help transfer enzymes responsible for production of signaling molecules including, but not limited to cAMP and cGMP-AMP may be included in the invention. As noted above, in certain embodiments the invention may include systems, methods and compositions for an improved system for the encapsulation and delivery of target ribonucleic acid or therapeutic RNA molecules to recipient cells through secreted fusogenic ectosome vesicles. In this preferred embodiment, a gectosome may be generated from a donor cell that may be configured to encapsulate protein-RNA complexes to target suppression of gene of interests by RNAi.

As noted above, Transducing nucleic acids or proteins into live cells to alter cellular function is crucial for studying gene function in the research space and growing increasingly more consequential to therapeutics due to the explosion of biologics as a compelling therapeutic modality. Viral-mediated gene transfer for overexpression, RNA interference, and gene editing works well for research but poses safety concerns for therapeutics. For decades, liposomes have been the preferred delivery vesicle for drugs and other cargo of interest. Despite their intense research development, major barriers including low stability, short circulation life, endosome degradation, high toxicity in vivo, inefficient loading for hydrophobic drugs, and difficulty in targeting remain to be overcome. EVs are heterogeneous nano-sized membrane vesicles constantly released by all cell-types. EVs have been classified either as exosomes or microvesicles, also known as ectosomes. Microvesicles are formed and released by budding from the cell's plasma membrane and are 150-1,000 nm in diameter. Exosomes are smaller vesicles of 40-150 nm in diameter and originate from endosomal compartments known as multivesicular bodies. While it has been well documented that exosomes can encapsulate small RNAs, its capability of carrying larger mRNA is still unproven. Furthermore, active loading of EVs with pre-determined cargoes and purifying them to homogeneity are required for development of EVs as therapeutics.

As generally described herein, the inventive technology described herein includes novel embodiment for making programmable, highly fusogenic microvesicles, or gectosomes which we call, as vehicles for the dose-controlled delivery of biologics. Gectosomes may include microvesicles decorated with vesicular stomatitis virus G protein (VSV-G), a viral glycoprotein that stimulates: 1) outward budding of vesicles at the plasma membrane of host cells; 2) internalization of vesicles into target cells; 3) efficient cargo release from endosomes. Due to proteins like VSV-G, viruses are proficient in delivering macromolecules to intracellular space. Borrowing from mechanisms of viral delivery, the gectosomes of the invention are configured to encapsulate predetermined proteins and nucleic acids through a simple complementation process. The pH-sensitive split GFP serves as the tether between VSV-G and the desired cargo proteins, which allows for efficient purification of cargo-loaded gectosomes using fluorescence-activated cell sorting after fluorescent gectosomes are formed during shedding to the extracellular space. During uptake of gectosomes by target cells, VSV-G catalyzes low pH-induced membrane fusion between gectosomes and endosomes, resulting in cargo release to the cytosol. The gectosomes of the invention, can execute efficient RNAi, CRISPR, and RNA ablation in target cells or CRISPR in live animal tissues. Gectosome technology harnesses the unique biological properties of VSV-G, the competitive binding principle to realize active loading of specific cargo, pH sensitivity, and fluorescence upon complementation to build a vehicle that can deliver cell modifying solutions.

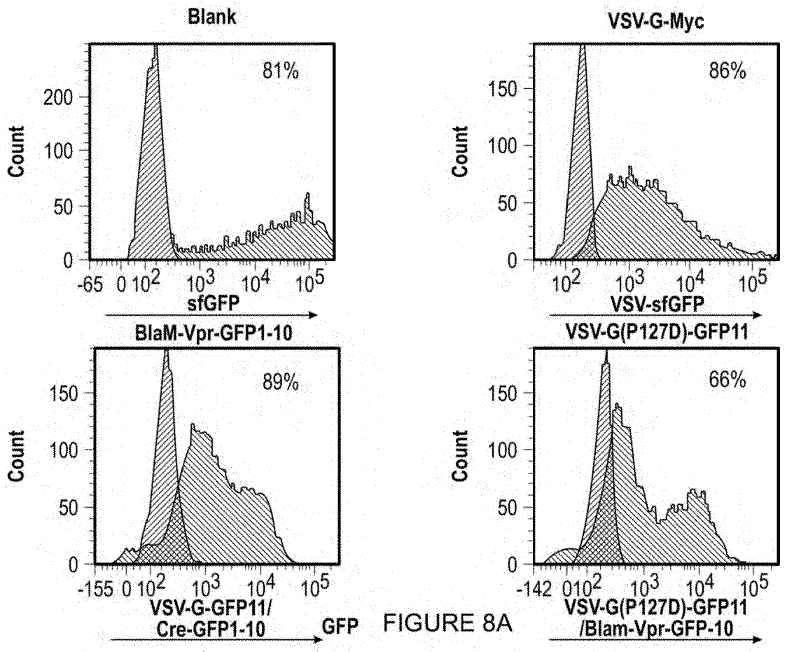

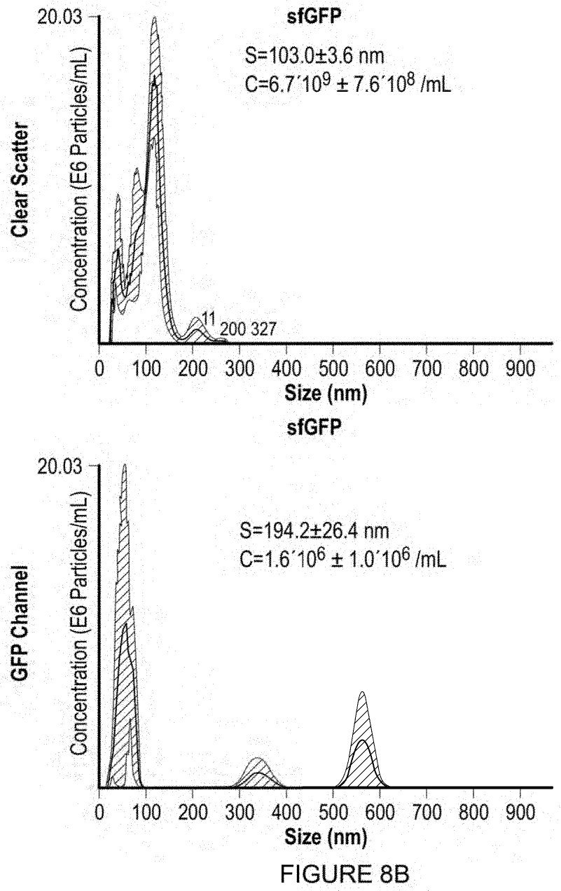

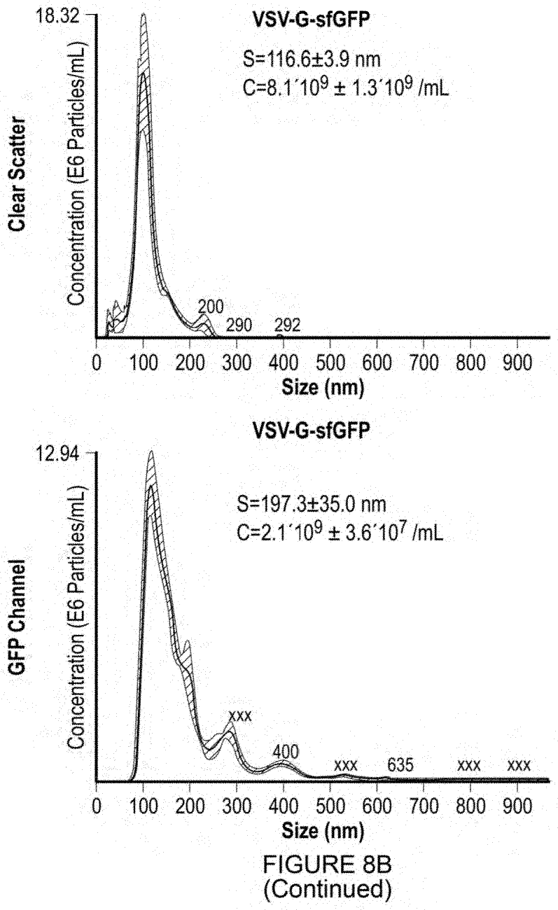

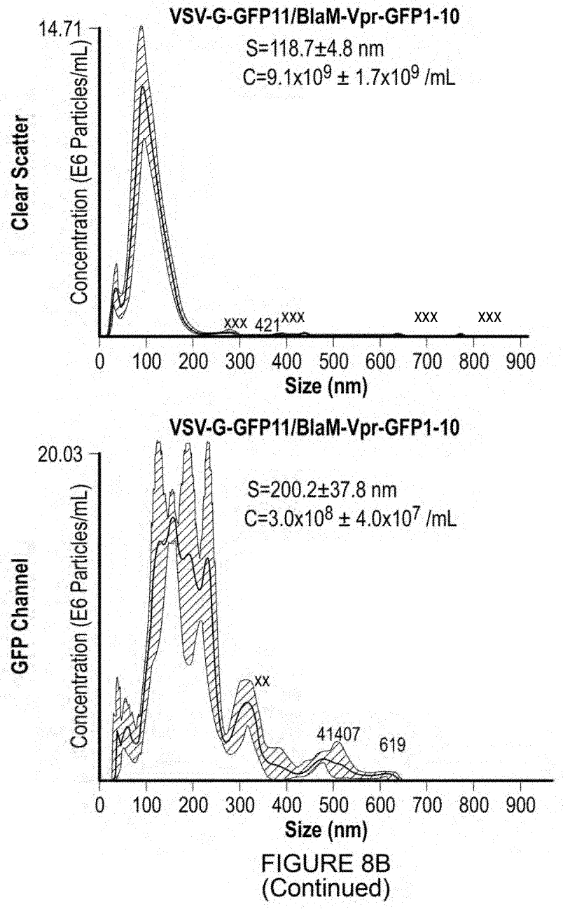

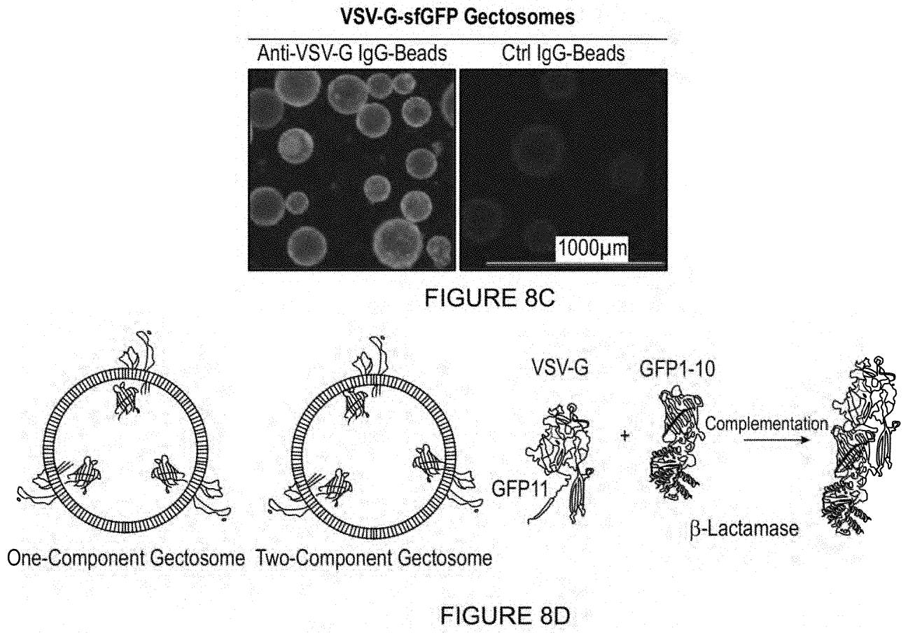

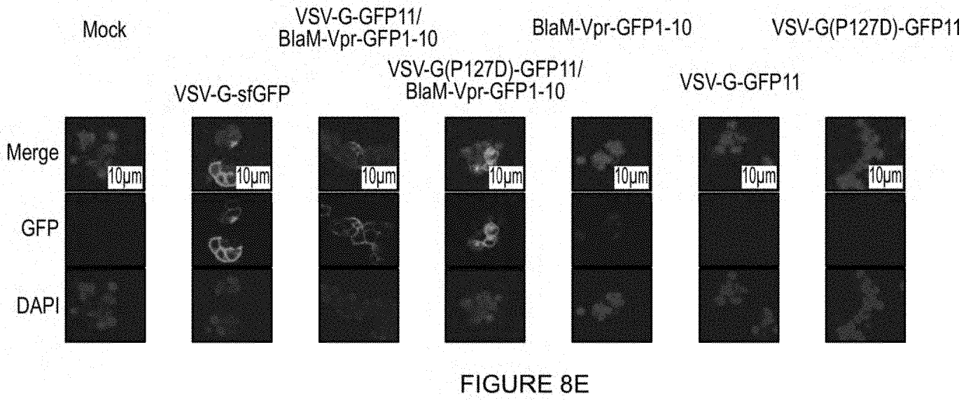

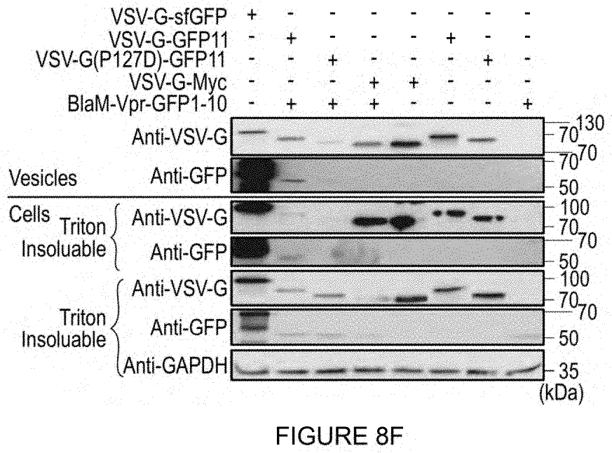

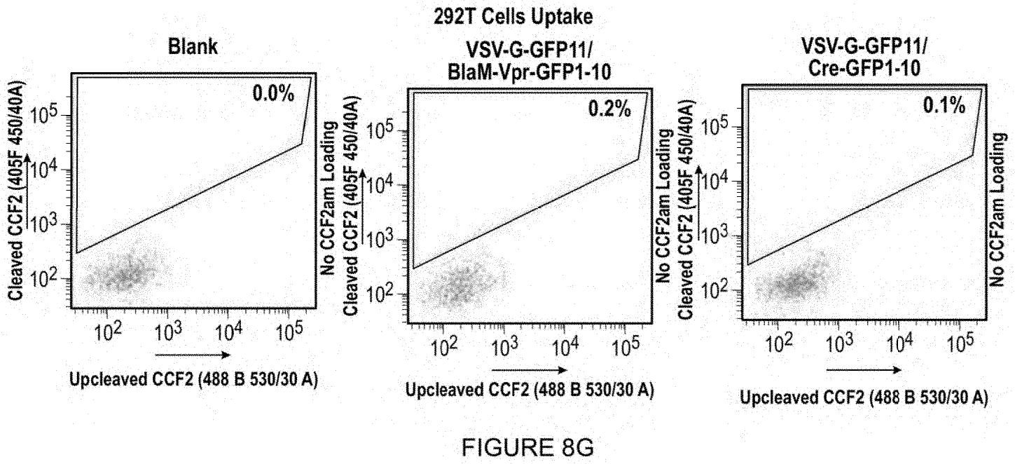

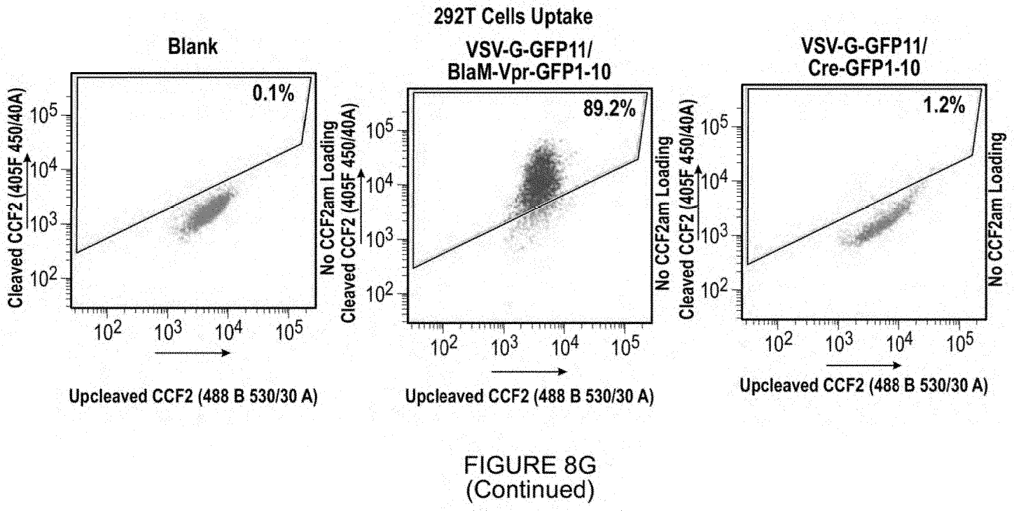

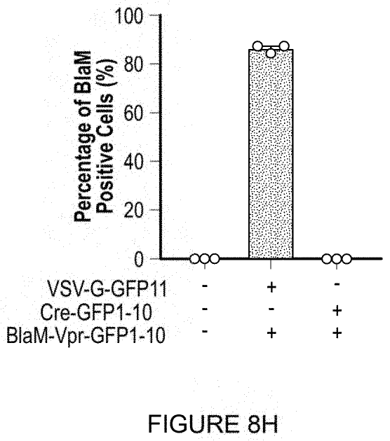

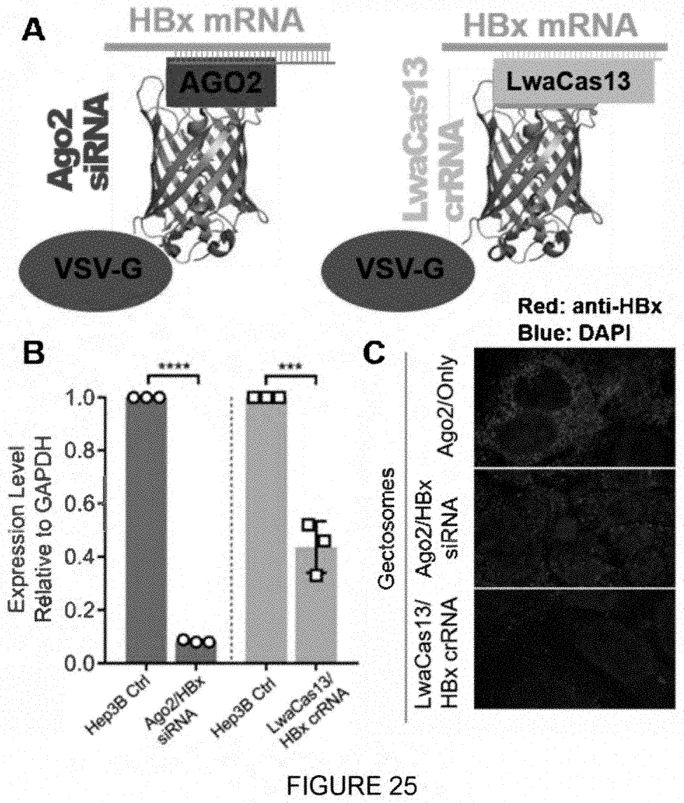

As shown in FIG. 25, in one specific embodiment, AGO2 or LwaCas13, a known essential components of the RNA-induced silencing complex (RISC) that binds small interfering RNAs (siRNAs) and other noncoding RNA including microRNAs (miRNAs) and Piwi-interacting RNAs (piRNAs), may be fused with a split components system, such as GFP1-10 and co-introduced with VSV-G-GFP11 fusion protein along with a target interfering RNA molecule, such as a short-hairpin RNA (shRNA). In this embodiment, the GFP1-10-AGO2 or a GFP-10-LwaCas13 construct may be co-introduced with VSV-G-GFP11 and a target interfering RNA (RNAi), such as a hpRNA, to a recipient cell through direct transfection, for example in an in vitro model. In alternative embodiments, the GFP1-10-AGO2 or LwaCas13 construct may be co-introduced with VSV-G-GFP11 and a target shRNA or other interfering RNA, such as a CRISPR RNA (crRNA) through the introduction of programmable gectosomes from a donor cell to a recipient cell, in an in vitro or in vivo system as generally outlined in FIGS. 8D and 1D. In each of the embodiments described above, the target RNAi molecule, such as a shRNA may be configured to inhibit expression of a specific endogenous gene in the target cell. Alternatively, in certain preferred embodiments, the target RNAi molecule, such as a shRNA may be configured to inhibit expression of a specific exogenous gene, such as an essential bacterial or pathogen gene or other transgene.

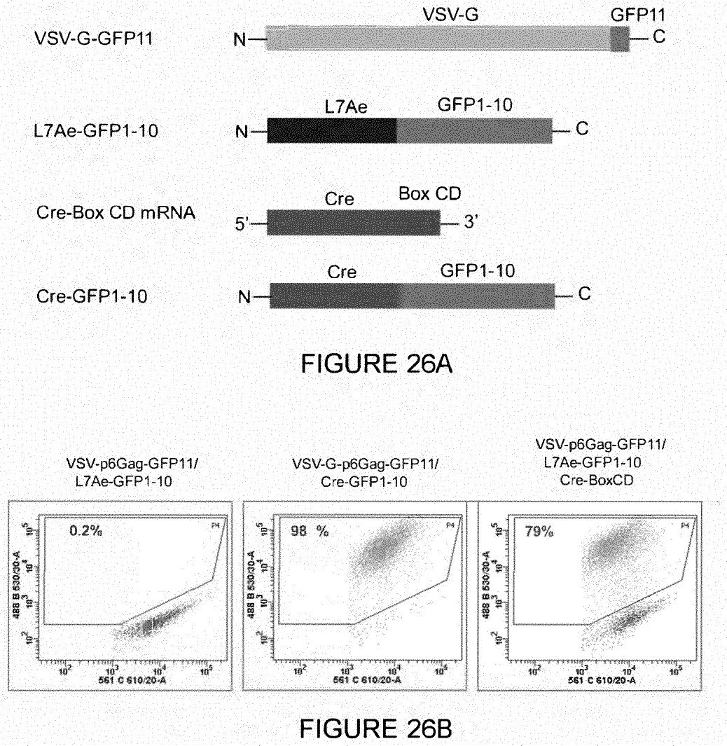

In another embodiment, a target mRNA molecule for a select peptide, may be delivered to a target cell through the gectosomes of the invention. As shown in FIG. 26, in one preferred embodiment a target peptide, such as L7Ae, having an RNA binding domain/motif may be coupled with a component of a split components system, such as GFP1-10. This fusion peptide may be co-expressed with a second fusion peptide having a membrane-binding motif, such as a VSV-G peptide that is coupled with a complementary component of the split component system of the first fusion peptide. A target RNA molecule may further be co-expressed with the first and second fusion peptides and may bind to the RNA binding domain of the target peptide domain.

In the preferred embodiment shown in FIG. 26, a target mRNA may include a coding region configured to be coupled with BoxCD binding domain that may interact with the RNA binding domain of a target peptide, such as L7Ae. Again, as shown in FIG. 26, a CRE mRNA having a BoxCD binding domain may bind to a corresponding BoxCD RNA binding domain of the target protein L7Ae. The L7Ae-GFP-1-10 may complement with a corresponding split protein of the VSV-G-GFP11 fusion peptide that is anchored to the cell membrane from which an EV can be formed as generally described herein. In this configuration, the CRE mRNA is loaded into the gectosome in a producing cell and may further be isolated and/or be introduced to a target call in vitro or in vivo, such that the mRNA is introduced into the intracellular compartment of the target cell and subsequently translated.

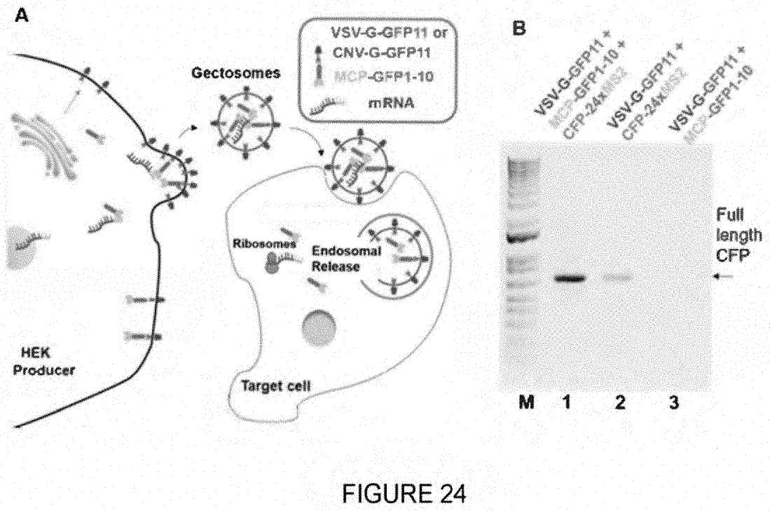

In another embodiment, mRNA molecules can be incorporated into gectosomes via active loading of gectosomes and detected in secreted gectosomes. For example, as shown in FIG. 24, in one embodiment, the inventions describes a two-hybrid gectosome system for mRNA loading and intercellular mRNA transfer. In this embodiment, a fusion peptide containing a MS2-coat protein and a component of a split components system may be co-expressed with a second fusion protein having a VSV-G peptide fused with a complementary components of the split component system, in this instance a split GFP system. As shown in FIG. 24, the VSV-G-GFP-11 fusion protein is anchored to a cell membrane that forms an extracellular vesicle (EV). The reporter RNA molecule binds to the MS2-coat protein target peptide and the GFP-1-10 portion of the split GFP system binds to is corresponding GFP-11 components thereby loading the mRNA bound target molecule into said EV forming a gectosome for delivery of the mRNA molecule to a target cell.

The invention further includes systems, methods and compositions for an improved system for the encapsulation and delivery of target genome-editing molecules to recipient cells through secreted fusogenic ectosome vesicles, such as gectosomes. In one preferred embodiment, the invention includes a programmable fusogenic ectosome vesicle, such as a gectosome, that is configured to selectively encapsulate and deliver specific genome-editing proteins to a recipient cell in a predetermined manner. Examples, may include, but not be limited to: Meganucleases (MGN), Zinc-Finger Nucleases (ZFN), Transcription Activator-Like Effector Nucleases (TALENs) and the like.

In a preferred embodiment, the inventive technology may include the systems, methods and compositions for the generation of secreted fusogenic vesicles that contain Cas9 and/or Cas13, or other genome editing proteins related to the genome editing process known as Clustered Regularly Interspaced Short Palindromic Repeats (CRISPR). In this preferred embodiment, a programmable fusogenic ectosome vesicle, such as a gectosome, may be configured to selectively encapsulate and deliver CRISPR ribonucleoproteins (RNP) to a target cell and mediate genome editing. In one specific embodiment, Cas9/sgRNA RNP, a known essential component of CRISPR genome editing, may be fused with tag, such as split complement protein system, such as GFP1-10 and co-introduced with VSV-G-GFP11. In this embodiment, the GFP1-10-Cas9/sgRNA RNP construct may be co-introduced with VSV-G-GFP11 to a recipient cell through direct transfection, for example in an in vitro model. In alternative embodiments, the GFP1-10-Cas9/sgRNA RNP construct may be co-introduced with VSV-G-GFP11 through the introduction of programmable gectosome from a donor cell to a recipient cell, in an in vitro or in vivo system as generally outline in FIG. 1D. In each of the embodiments described above, the sgRNA, or single guide RNA molecule, may be configured to target a specific endogenous gene in the target. Alternatively, in certain preferred embodiments, the target sgRNA molecule may be configured to inhibit expression of a specific exogenous gene, such as an essential bacterial or pathogen gene or other transgene.

The inventive technology may further include the generation of engineered fusogenic secreted vesicles through the action of human Gag-like proteins. In this embodiment, one or more human Gag-like endogenous proteins may be coupled with an interacting moiety, such as GFP11 or a similar tag that can be recognized by encapsulated proteins that bind proteins/peptides, nucleic acids or other small molecules as generally described herein.

The invention may further include systems and methods and compositions for the use of engineered fusogenic secreted vesicles, such as gectosomes for the treatment of a disease condition. Examples may include, but not be limited to treatment and/or prevention of cancer, autoimmune conditions, vaccines, and organ and/or cell transplant rejection. In one preferred embodiment, a therapeutically effective amount of engineered fusogenic secreted vesicles, as generally described herein, may be introduced to a recipient cell exhibiting a disease condition, such that the action of the engineered fusogenic secreted vesicles may alleviate and or prevent a disease condition. In additional embodiments, engineered fusogenic secreted vesicles, such as gectosomes, may be used to increase host immunity and/or metabolic fitness or even replace missing or defective cell pathways in a recipient cell.

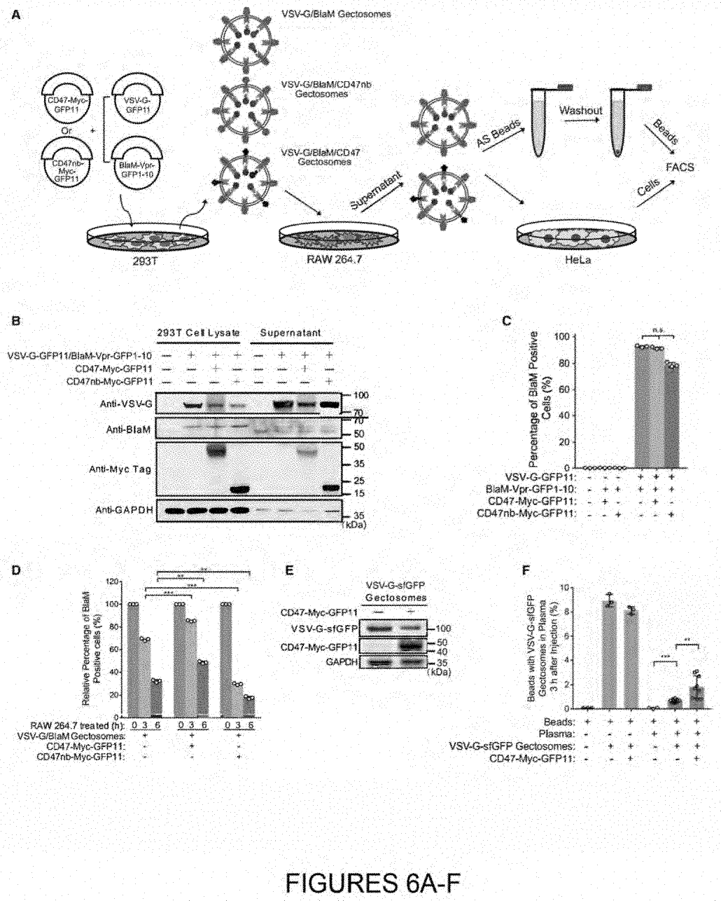

In additional embodiment, the invention may include the generation of high-efficiency persistent gectosomes that may resist clearance by the immune system, for example through the expression of surface biomarkers that prevent immune system clearances. As shown in FIG. 6A, in one preferred embodiment, such a high-efficiency persistent gectosomes that may resist clearance by the macrophages through the overexpression and presentation of CD47 proteins on the surface of the gectosomes.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference. The terminology used herein is for describing particular embodiments and is not intended to be limiting. As used herein, the singular forms “a,” “and” and “the” include plural referents unless the content and context clearly dictate otherwise. Thus, for example, a reference to “a” or “the” marker may include a combination of two or more such markers. Unless defined otherwise, all scientific and technical terms are to be understood as having the same meaning as commonly used in the art to which they pertain. For the purposes of the present invention, the following terms are defined below.

As used generally herein, VSV-G-containing EVs are generally referred to as “gectosomes.” In other embodiments, EVs containing one or more fusogenic proteins may also be referred to as an “gectosomes.”

As used herein, the term “p6^{Gag}” refer to an HIV protein comprising a viral L domain. p6 also refers to proteins that comprise artificially engineered L domains including, for example, L domains comprising a series of L motifs. An exemplary HIV-p6^{Gag} is SEQ ID NO: 2. The term “Gag protein” or “Gag polypeptide” refers to a polypeptide having Gag activity and preferably comprising an L (or late) domain. Exemplary Gag proteins motif include a motif such as PXXP, PPXY, PXXY, YXXL, RXXPXXP, RPDPTAP, RPLPVAP, RPEPTAP, PTAPPEY, PTAPPEE and/or RPEPTAPPEE. An exemplary HIV-1 Gag protein Typically, an HIV Gag protein comprises a p6^{Gag} protein motif/sequence SEQ ID NO: 2.

As used herein, the term “Split Fluorescent Proteins (SFPs)” means a system having are composed of multiple fragments of the eleven anti-parallel outer β-strands and one inner α-strand of a fluorescent protein. Individually the fragments are not fluorescent, hut, when complemented, form a functional fluorescent molecule. Typically, the SPF includes a first fragment known as a “SFP detector” that includes nine or ten contiguous β-strands and the α-strand of the fluorescent protein or a circular permutant thereof, and one or two separate fragments known as the “SFP tag(s)” that include the remaining β-strand or strands. Some tripartite SFP systems are known, which include three separate proteins that can form a fluorescent protein. For example, a tripartite split-Green Fluorescent Protein (split-GFP) system can include an SFP detector including GFP β-strands 1-9 (GFP1-9), a first SFP tag including GFP β-strand 10 (GFP10), and a third. SFP tag including GFP β-strand 11 (GFP11). The GFP10 and GFP11 tags can be placed on unrelated polypeptide sequences and detected using the GFP1-9 detector.

As used herein, the term “fusogenic” refers to the fusion of the plasma membrane of the microvesicles to the membrane of the target cell. A “fusogenic vesicle” may include a vesicle that incorporates a fusogenic protein. A peptide may be “fusogenic” or a “fusion peptide” is it has a membrane-fusion moiety or domain.

The term “endogenous” protein means that said protein is not expressed from a gene naturally found in the genome of a eukaryotic cell.

The term “exogenous” protein means that said protein is not expressed from a gene naturally found in the genome of a eukaryotic cell.

The term “pharmaceutically acceptable” or “pharmacologically acceptable” as used herein, refer to molecular entities and compositions that do not produce adverse, allergic, or other untoward reactions when administered to an animal or a human.
The term, “pharmaceutically acceptable carrier” as used herein, includes any and all solvents, or a dispersion medium including, but not limited to, water, ethanol, a polyol (for example, glycerol, propylene glycol, and liquid polyethylene glycol, and the like), suitable mixtures thereof, and vegetable oils, coatings, isotonic and absorption delaying agents, liposomes, commercially available cleansers, and the like. Supplementary bioactive ingredients also can be incorporated into such carriers.

The term “fusiogenic protein” or “fusion protein” refers to a protein, and preferably a viral protein that can induce the fusion of the plasma membrane derived envelope of the VLP to the membrane of the recipient cell. It is this mechanism that results in entry of the proteinaceous component of the VLP to the cytosol. The envelope glycoproteins of RNA viruses and retroviruses are well known to bind cell receptors and induce this fusion. Accordingly, these proteins are responsible for the infectivity of these viruses. Other examples of fusiogenic proteins include, but are not limited to, influenza haemagglutinin (HA), the respiratory syncytial virus fusion protein (RSVFP), the E proteins of tick borne encephalitis virus (TBEV) and dengue fever virus, the E1 protein of Semliki Forest virus (SFV), the G proteins of rabies virus and vesicular stomatitis virus (VSV) and baculovirus gp64. Functionally equivalent fragments or derivatives of these proteins may also be used. The functionally equivalent fragments or derivatives will retain at least 50%, more preferably at least 75% and most preferably at least 90% of the fusiogenic activity of the wild-type protein.

Particularly preferred is the envelope glycoprotein from the Vesicular Stomatitis Virus (VSV-G) (SEQ ID NO. 1). VSV-G has high fusiogenic activity and virtually all mammalian cells can bind VSV-G, via the carbohydrate moiety of their plasma membrane glycoproteins. Without wishing to be being bound by theory, the molecular mechanism of VSV-G-cell surface interaction consists of attachment, followed by a step of membrane fusion between the membrane of the cell and the viral envelope. This process has been well documented for the influenza virus haemagglutinin and host cell plasma membranes.

Any convenient cell capable of producing microvesicles may be utilized. In some instances, the cell is a eukaryotic cell. Cells of interest include eukaryotic cells, e.g., animal cells, where specific types of animal cells include, but are not limited to: insect, worm, avian or mammalian cells. Various mammalian cells may be used, including, by way of example, equine, bovine, ovine, canine, feline, murine, non-human primate and human cells. Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells). Specific cells of interest include, but are not limited to: mammalian cells, e.g., HEK-293 and HEK-293T cells, COS7 cells, Hela cells, HT1080, 3T3 cells etc.; insect cells, e.g., High5 cells, Sf9 cells, Sf21 and the like. Additional cells of interest include, but are not limited to, those described in US Publication No. 20120322147, the disclosure of which cells are herein incorporated by reference.

In specific embodiments, the present invention also relates to an in vitro method for delivering a protein of interest into a target cell by contacting said target cell with an engineered fusogenic secreted vesicles, such as a gectosome, of having a cargo of a target protein of other molecule of interest. Examples of target cells are common laboratory cell lines such Hela cells and derivatives, HEK293 cells, HEK293T cells, NIH3T3 cells and derivatives, HepG2 cells, HUH7 cells and derivatives, small lung cancer cells, Caco-2 cells, L929 cells, A549 cells, MDCK cells, THP1 cells, U937 cells, Vero cells and PC12 cells; human hematopoietic cells CD34+ purified from bone marrow, from blood, from umbilical cord; Dendritic Cells (DCs) differentiated from blood monocytes or from CD34+ cells; primary human cells purified from blood including T-cells (CD8 and CD4), B-cells (including memory B-cells), Mast cells, macrophages, DCs, NK-cells; primary murine cells purified from blood including T-cells (CD8 and CD4), B-cells (including memory B-cells), Mast cells, macrophages, DCs, NK-cells; primary human fibroblasts including MRCS cells, IMR90 cells; primary murine fibroblasts and Embryonic Stem cells (ES) from human, murine, rat, chicken, rabbit origin.

As summarized above, aspects of the invention include methods of introducing a protein into a target cell through introduction of an engineered fusogenic secreted vesicle, such as a gectosome. Such methods include contacting the target cell with a engineered fusogenic secreted vesicles, e.g., as described above, where the engineered fusogenic secreted vesicles may be present in a composition of a population (for example where the number of engineered fusogenic secreted vesicles ranges from 10³ to 10¹⁶, such as 10⁴ to 10¹³, including as 10⁴ to 10⁹), under conditions sufficient for the micro-vesicle to fuse with the target cell and deliver the target protein or molecule contained in the engineered fusogenic secreted vesicles into the cell. Any convenient protocol for contacting the cell with the engineered fusogenic secreted vesicles may be employed. The particular protocol that is employed may vary, e.g., depending on whether the target cell is in vitro or in vivo. For in vitro protocols, target cells may be maintained with donor cells configured to generate engineered fusogenic secreted vesicles and/or isolated engineered fusogenic secreted vesicles in a suitable culture medium under conditions sufficient for the engineered fusogenic secreted vesicles to fuse with the target cells.

As noted above, target proteins may include research proteins which may include proteins whose activity finds use in a research protocol and/or as a therapeutic protocol. As such, research proteins are proteins that are employed in an experimental procedure. The research protein may be any protein that has such utility, where in some instances the research protein is a protein domain that is also provided in research protocols by expressing it in a cell from an encoding vector. Examples of specific types of research proteins include, but are not limited to: transcription modulators of inducible expression systems, members of signal production systems, e.g., enzymes and substrates thereof, hormones, prohormones, proteases, enzyme activity modulators, perturbimers and peptide aptamers, antibodies, modulators of protein-protein interactions, genomic modification proteins, such as CRE recombinase, meganucleases, Zinc-finger nucleases, CRISPR/Cas-9 nuclease, TAL effector nucleases, etc., cellular reprogramming proteins, such as Oct 3/4, Sox2, Klf4, c-Myc, Nanog, Lin-28, etc., and the like.

Target proteins may be diagnostic proteins whose activity finds use in a diagnostic protocol. As such, diagnostic proteins are proteins that are employed in a diagnostic procedure. The target diagnostic protein may be any protein that has such utility. Examples of specific types of diagnostic proteins include but are not limited to: members of signal production systems, e.g., enzymes and substrates thereof, labeled binding members, e.g., labeled antibodies and binding fragments thereof, peptide aptamers and the like.

Target proteins of interest further include therapeutic proteins. Therapeutic proteins of interest include without limitation, hormones and growth and differentiation factors including, without limitation, insulin, glucagon, growth hormone (GH), parathyroid hormone (PTH), growth hormone releasing factor (GHRF), follicle stimulating hormone (FSH), luteinizing hormone (LH), human chorionic gonadotropin (hCG), vascular endothelial growth factor (VEGF), angiopoietin, angiostatin, granulocyte colony stimulating factor (GCSF), erythroproietin (EPO), connective tissue growth factor (CTGF), basic fibroblast growth factor (bFGF), acidic fibroblast growth factor (aFGF), epidermal growth factor (EGF), transforming growth factor.alpha. (TGFα), platelet-derived growth factor (PDGF), insulin growth factors I and II (IGF-I and IGF-II), any one of the transforming growth factor β-superfamily, including TGFβ, activins, inhibins, or any of the bone morphogenic proteins (BMP) including BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase. Target proteins of interest further include, but are not limited to: fibrinolytic proteins, including without limitation, urokinase-type plasminogen activator (u-PA), and tissue plasminogen activator (tpA); procoagulant proteins, such as Factor Vila, Factor VIII, Factor IX and fibrinogen; plasminogen activator inhibitor-1 (PAI-1), von Willebrand factor, Factor V, ADAMTS-13 and plasminogen for use in altering the hemostatic balance at sites of thrombosis; etc. Also, of interest as target proteins are transcription factors such as jun, fos, max, mad, serum response factor (SRF), AP-1, AP2, myb, MyoD, myogenin, ETS-box containing proteins, TFE3, E2F, ATF1, ATF2, ATF3, ATF4, ZF5, NFAT, CREB, HNF4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins. Also of interest as target proteins are carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylase, hepatic phosphorylase, phosphorylase kinase, glycine decarboxylase, H-protein, T-protein, a cystic fibrosis transmembrane regulator (CFTR) sequence, and a dystrophin cDNA sequence.

Further included are methods for improving the efficacy of a disease therapy by administering or introducing to a subject, in vivo or in vitro a therapeutically effective amount of engineered fusogenic secreted vesicles, such as gectosomes, configured to have a therapeutic effect. In this context, the term “effective” or “effective amount” or “therapeutically effective amount” is to be understood broadly to include reducing or alleviating the signs or symptoms of a disease, improving the clinical course of the disease, or reducing any other objective or subjective indicia of the disease.

The term “nucleic acid” as used herein, refers to a polymer of ribonucleotides or deoxyribonucleotides. Typically, “nucleic acid or “nucleic acid agent” polymers occur in either single or double-stranded form but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Exemplary analogs include, without limitation, phosphorothioates, phosphoramidates, methyl phosphonates, chiral-methyl phosphonates, 2-O-methyl ribonucleotides, and peptide-nucleic acids (PNAs). “DNA”, “RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein.

The terms “engineered” or “programmable” comprises fusogenic secreted vesicles that have been modified so as to be non-naturally occurring and that may be configured to load and/or deliver target molecules.

As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

In another embodiment, the invention provides polynucleotides that have substantial sequence similarity to a target polynucleotide molecule that is described herein. Two polynucleotides have “substantial sequence identity” when there is at least about 70% sequence identity, at least about 80% sequence identity, at least about 90% sequence identity, at least about 95% sequence identity or at least 99% sequence identity between their amino acid sequences or when the polynucleotides are capable of forming a stable duplex with each other under stringent hybridization conditions. Such conditions are well known in the art. As described above with respect to polypeptides, the invention includes polynucleotides that are allelic variants, the result of SNPs, or that in alternative codons to those present in the native materials as inherent in the degeneracy of the genetic code. Moreover, disclosure of a nucleotide sequence encompasses all corresponding amino acid sequences that it could produce during translation. Conversely, disclosure of an amino acid sequence encompasses all corresponding nucleotide sequences, including DNA and RNA, that correspond could give rise to the peptide considering the redundant nature of the genetic code as described herein.

As used herein, the phrase “expression,” “gene expression” or “protein expression,” such as the level of includes any information pertaining to the amount of gene transcript or protein present in a sample, in a cell, in a patient, secreted in a sample, and secreted from a cell as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc. The term “expression levels” refers to a quantity reflected in or derivable from the gene or protein expression data, whether the data is directed to gene transcript accumulation or protein accumulation or protein synthesis rates, etc.

As used herein, an engineered fusogenic secreted vesicles, such as gectosome, is referred to as “isolated” when it has been separated from at least one component with which it is naturally associated.

Polypeptides encoded by a target molecule genes that may be targeted for expression inhibition, for example through an RNAi mediated process herein may reflect a single polypeptide or complex or polypeptides. Accordingly, in another embodiment, the invention provides a polypeptide that is a fragment, precursor, successor or modified version of a protein target molecule described herein. In another embodiment, the invention includes a protein target molecule that comprises a foregoing fragment, precursor, successor or modified polypeptide. As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of poly- or oligonucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g., 5, 6, 7, 8, 9, 10, 11 or 12 or more amino acids long, including each integer up to the full length of the polypeptide). Those skilled in the art are well versed in methods for selecting antigenic fragments of proteins.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “introducing,” “administered” or “administering”, as used herein, refers to any method of providing a composition of engineered fusogenic secreted vesicles to a patient such that the composition has its intended effect on the patient. In one embodiment, engineered fusogenic secreted vesicles may be introduced to a patient in vivo, while in other alternative embodiments, engineered fusogenic secreted vesicles may be introduced to subject cells in vitro which may then be administered to a patient in vivo.

The term “patient” as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “cell” as used herein, may include a cell or cells in an in vivo system, such as a subject or patient, or an in vitro system, such as a cell-line or cell-based assay.

The term “coupled” may include direct and indirect connections. In one preferred embodiment, it may mean fused, as in a fusion or chimera protein or molecule.

The term “subject” as used herein refers to a vertebrate, preferably a mammal, more preferably a primate, still more preferably a human. Mammals include, without limitation, humans, primates, wild animals, feral animals, farm animals, sports animals, and pets.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens. As noted above, the terms protein and peptide also include protein fragments, epitopes, catalytic sites, signaling sites, localization sites and the like. A peptide or protein may further be a fusion peptide, which a used herein means a peptide having at least a first and second domain or moiety. As described herein, in certain embodiment various peptides, including fusion peptides or oligonucleotides, such as RNA molecules may be co-expressed. In some embodiments the elements may be co-expressed from a single expression vector having one or more expression cassettes, or from separate expression vectors having one or more expression cassettes. Such expression may also be the result of transient or stable transformation of a cell.

Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “expression cassette” refers to a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The peptides of the invention of the present invention may be chimeric. The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus.

As used herein, a promoter region or promoter element refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated.

As used herein, the term “antibody” refers to an immunoglobulin molecule capable of binding an epitope present on an antigen. The term is intended to encompass not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity.

A further aspect of the invention relates to the use of DNA editing compositions and methods to inhibit, alter, disrupt expression and/or replace one or more target genes. In various embodiments, one or more target genes may be altered through CRISPR/Cas-9, TALAN or Zinc (Zn2+) finger nuclease systems which may be loaded and delivered through engineered fusogenic secreted vesicles to a recipient cell.

In some embodiments, the agent for altering gene expression is CRISPR-Cas9, or a functional equivalent thereof, together with an appropriate RNA molecule arranged to target one or more target genes. For example, one embodiment of the present invention may include the introduction of one or more guide RNAs (gRNAs) to be utilized by CRISPR/Cas9 system to disrupt, replace, or alter the expression or activity of one or more target genes in a recipient cell. In this context, the gene-editing CRISPR/cas-9 technology is an RNA-guided gene-editing platform that makes use of a bacterially derived protein (Cas9) and a synthetic guide RNA to introduce a double strand break at a specific location within the genome. Editing is achieved by transfecting a cell or a subject with the Cas9 protein along with a specially designed guide RNA (gRNA) that directs the cut through hybridization with its matching genomic sequence. By making use of this technology, it is possible to introduce specific genetic alterations in one or more target genes. In some embodiments, this CRISPR/cas-9 may be utilized to replace one or more existing wild-type genes with a modified version, while additional embodiments may include the addition of genetic elements that alter, reduce, increase or knock-out the expression of a target gene in a recipient cell.

In some embodiments, the agent for altering gene expression is a zinc finger, or zinc finger nuclease or other equivalent. The term “zinc finger nuclease” or “zinc fingers” as used herein, refers to a nuclease comprising a nucleic acid cleavage domain conjugated to a binding domain that comprises a zinc finger array. In some embodiments, the cleavage domain is the cleavage domain of the type II restriction endonuclease FokI. Zinc finger nucleases can be designed to target virtually any desired sequence in a given nucleic acid molecule for cleavage, and the possibility to design zinc finger binding domains to bind unique sites in the context of complex genomes allows for targeted cleavage of a single genomic site in living cells, for example, to achieve a targeted genomic alteration of therapeutic value. Targeting a double-strand break to a desired genomic locus can be used to introduce frame-shift mutations into the coding sequence of a gene due to the error-prone nature of the non-homologous DNA repair pathway. Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo Colo. (May 1991). “Zinc finger-DNA recognition: crystal structure of a Zif268-DNA complex at 2.1 A”. Science 252 (5007): 809-17, the entire contents of which are incorporated herein). In some embodiments, separate zinc fingers that each recognize a 3 base pair DNA sequence are combined to generate 3-, 4-, 5-, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6-, or 8-zinc finger arrays. In some embodiments, bacterial or phage display is employed to develop a zinc finger domain that recognizes a desired nucleic acid sequence, for example, a desired nuclease target site of 3-30 bp in length.

Zinc finger nucleases, in some embodiments, comprise a zinc finger binding domain and a cleavage domain fused or otherwise conjugated to each other via a linker, for example, a polypeptide linker. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a FokI cleavage domain, and one monomer comprising zinc finger domain B conjugated to a FokI cleavage domain. In this non-limiting example, zinc finger domain A binds a nucleic acid sequence on one side of the target site, zinc finger domain B binds a nucleic acid sequence on the other side of the target site, and the dimerize FokI domain cuts the nucleic acid in between the zinc finger domain binding sites.

In some embodiments, the agent for altering the target gene is a TALEN system or its equivalent. The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a FokI domain. A number of modular assembly schemes for generating engineered TALE constructs have been reported (Zhang, Feng; et. al. (February 2011). Those of skill in the art will understand that TALE nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene or allele associated with a disease or disorder or a biological process, or one or more target genes. In some embodiments, delivery of the TALEN to a subject confers a therapeutic benefit to the subject, such as reducing, ameliorating or eliminating disease condition in a patient.

In some embodiments, the target gene of a cell, tissue, organ or organism is altered by a nuclease delivered to the cell via a strategy or method disclosed herein, e.g., CRISPR/cas-9, a TALEN, or a zinc-finger nuclease, or a plurality or combination of such nucleases. In some embodiments, a single- or double-strand break is introduced at a specific site within the genome by the nuclease, resulting in a disruption of the target genomic sequence.

In some embodiments, the target genomic sequence is a nucleic acid sequence within the coding region of a target gene. In some embodiments, the strand break introduced by the nuclease leads to a mutation within the target gene that impairs the expression of the encoded gene product. In some embodiments, a nucleic acid is co-delivered to the cell with the nuclease. In some embodiments, the nucleic acid comprises a sequence that is identical or homologous to a sequence adjacent to the nuclease target site. In some such embodiments, the strand break affected by the nuclease is repaired by the cellular DNA repair machinery to introduce all or part of the co-delivered nucleic acid into the cellular DNA at the break site, resulting in a targeted insertion of the co-delivered nucleic acid, or part thereof. In some embodiments, the insertion results in the disruption or repair of the undesired allele. In some embodiments, the nucleic acid is co-delivered by association to a supercharged protein. In some embodiments, the supercharged protein is also associated to the functional effector protein, e.g., the nuclease. In some embodiments, the delivery of a nuclease to a target cell results in a clinically or therapeutically beneficial alteration of the function of a gene.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein, the term “RNAi molecules” “interfering RNA molecules” or “interfering RNA” or RNA molecules configured to mediate RNA interference generally refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNAi molecules include dsRNAs such as siRNAs, miRNAs and shRNAs, sgRNA, CRISPR RNA (crRNs). In one embodiment, the RNA silencing agent is capable of inducing RNA interference. In another embodiment, the RNA silencing agent is capable of mediating translational repression. As used herein, an RNA molecule or even RNAi molecule may further encompass lincRNA molecules as well as lncRNA molecules.

In some embodiments of the invention, the nucleic acid agent is a double stranded RNA (dsRNA). As used herein the term “dsRNA” relates to two strands of anti-parallel polyribonucleic acids held together by base pairing. The two strands can be of identical length or of different lengths, provided there is enough sequence homology between the two strands that a double stranded structure is formed with at least 60%, 70% 80%, 90%, 95% or 100% complementary over the entire length. According to an embodiment of the invention, there are no overhangs for the dsRNA molecule. According to another embodiment of the invention, the dsRNA molecule comprises overhangs. According to other embodiments, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. It will be noted that the dsRNA can be defined in terms of the nucleic acid sequence of the DNA encoding the target gene transcript, and it is understood that a dsRNA sequence corresponding to the coding sequence of a gene comprises an RNA complement of the gene's coding sequence, or other sequence of the gene which is transcribed into RNA.

The inhibitory RNA sequence can be greater than 90% identical or even 100% identical, to the portion of the target gene transcript. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60 degrees C. hybridization for 12-hours; followed by washing). The length of the double-stranded nucleotide sequences complementary to the target gene transcript may be at least about 18, 19, 21, 25, 50, 100, 200, 300, 400, 491, 500, 550, 600, 650, 700, 750, 800, 900, 1000 or more bases. In some embodiments of the invention, the length of the double-stranded nucleotide sequence is approximately from about 18 to about 530, or longer, nucleotides in length.

The present teachings relate to various lengths of dsRNA, whereby the shorter version i.e., x is shorter or equals 50 bp (e.g., 17-50), is referred to as siRNA or miRNA. Longer dsRNA molecules of 51-600 are referred to herein as dsRNA, which can be further processed for siRNA molecules. According to some embodiments, the nucleic acid sequence of the dsRNA is greater than 15 base pairs in length. According to yet other embodiments, the nucleic acid sequence of the dsRNA is 19-25 base pairs in length, 30-100 base pairs in length, 100-250 base pairs in length or 100-500 base pairs in length. According to still other embodiments, the dsRNA is 500-800 base pairs in length, 700-800 base pairs in length, 300-600 base pairs in length, 350-500 base pairs in length or 400-450 base pairs in length. In some embodiments, the dsRNA is 400 base pairs in length. In some embodiments, the dsRNA is 750 base pairs in length.

The term “siRNA” refers to small inhibitory RNA duplexes (generally between 17-30 base pairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC. It has been found that position of the 3′-overhang influences potency of a siRNA and asymmetric duplexes having a 3′-overhang on the antisense strand are generally more potent than those with the 3′-overhang on the sense strand. This can be attributed to asymmetrical strand loading into RISC, as the opposite efficacy patterns are observed when targeting the antisense transcript.

In certain embodiments, dsRNA can come from 2 sources; one derived from gene transcripts generated from opposing gene promoters on opposite strands of the DNA and 2) from fold back hairpin structures produced from a single gene promoter but having internal complimentary. For example, strands of a double-stranded interfering RNA (e.g., a siRNA) may be connected to form a hairpin or stem-loop structure (e.g., a shRNA). Thus, as mentioned, the RNA silencing agent may also be a short hairpin RNA (shRNA). The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. It will be recognized by one of skill in the art that the resulting single chain oligonucleotide forms a stem-loop or hairpin structure comprising a double-stranded region capable of interacting with the RNAi machinery.

As used herein, the phrase “microRNA (also referred to herein interchangeably as “miRNA”) or a precursor thereof” refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence, essentially complementary to the nucleotide sequence of the miRNA molecule. Typically, a miRNA molecule is processed from a “pre-miRNA,” or as used herein, a precursor of a pre-miRNA molecule by proteins, such as DCL proteins, and loaded onto a RISC complex where it can guide the cleavage of the target RNA molecules. Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides, which can adopt a secondary structure comprising an imperfect double stranded RNA stem and a single stranded RNA loop (also referred to as “hairpin”), and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g., between 30 and 50 nucleotides in length. The complementarity between the miRNA and its complement need not be perfect, and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand, which at its 5′ end, is the least involved in hydrogen bonding between the nucleotides of the different strands of the cleaved dsRNA stem, is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bonds, or G and U involving two hydrogen bonds is less strong that between G and C involving three hydrogen bonds.

Naturally occurring miRNA molecules may be comprised within their naturally occurring pre-miRNA molecules, but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

According to the present teachings, the dsRNA molecules may be naturally occurring or synthetic. The dsRNA can be a mixture of long and short dsRNA molecules such as, dsRNA, siRNA, siRNA+dsRNA, siRNA+miRNA, or a combination of same.

In a preferred embodiment, one or more nucleic acid agents are designed for specifically targeting a target gene of interest. It will be appreciated that the nucleic acid agent can be used to downregulate one or more target genes (e.g., as described in detail above). If a number of target genes are targeted, a heterogenic composition which comprises a plurality of nucleic acid agents for targeting a number of target genes is used. Alternatively, the plurality of nucleic acid agents is separately formulated. According to a specific embodiment, a number of distinct nucleic acid agent molecules for a single target are used, which may be used separately or simultaneously (i.e., co-formulation) applied.

For example, in order to silence the expression of an mRNA of interest, synthesis of the dsRNA suitable for use with some embodiments of the invention can be selected as follows. First, the mRNA sequence is scanned including the 3′ UTR and the 5′ UTR. Second, the mRNA sequence is compared to an appropriate genomic database using any sequence alignment software, such as the BLAST software available from the NCBI server (wwwdotncbidotnlmdotnihdotgov/BLAST/). Putative regions in the mRNA sequence which exhibit significant homology to other coding sequences are filtered out. Qualifying target sequences are selected as templates for dsRNA synthesis. Preferred sequences are those that have as little homology to other genes in the genome to reduce an “off-target” effect. It will be appreciated that the RNA silencing agent of some embodiments of the invention need not be limited to those molecules containing only RNA, but further encompasses chemically-modified nucleotides and non-nucleotides.

The terms “comprises”, “comprising”, are intended to have the broad meaning ascribed to them in U.S. Patent Law and can mean “includes”, “including” and the like.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1: Overview of Gectosome Production and Methods of Use Thereof

Here, the present inventors have developed a method for pharmacologically delivering bioactive proteins, RNA-interfering machinery, and Cas9/sgRNA complexes in vitro and in vivo utilizing the unique properties of the invention's fusogenic Gectosomes and an active cargo-loading strategy to achieve the highly efficient delivery of macromolecules to the interior of mammalian cells. In one embodiment, the present inventors found that active loading of Gectosomes via the split GFP system reduces vesicle heterogeneity by suppressing passive incorporation of cellular proteins, increases the specific activity of delivery, and enables purification of vesicles for cargo, thereby minimizing the undesirable effects bioactive contaminants. Biologics designed for modulating intracellular targets are challenging to develop as therapeutics due to their reduced ability to penetrate the cell and endosomal membranes. With the development of genome-editing technologies and therapeutic nucleic acids, intense efforts are devoted to addressing these delivery issues.

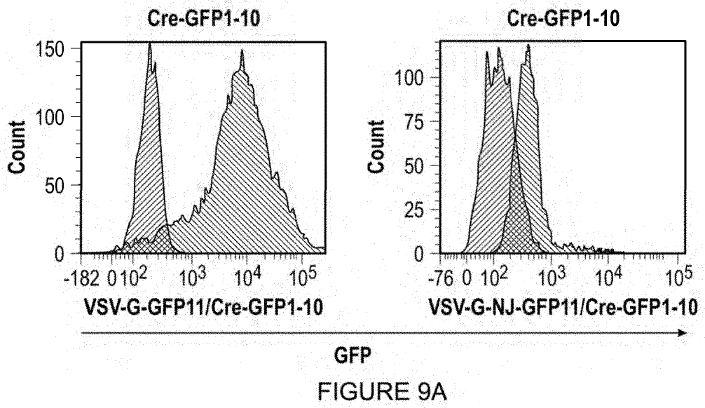

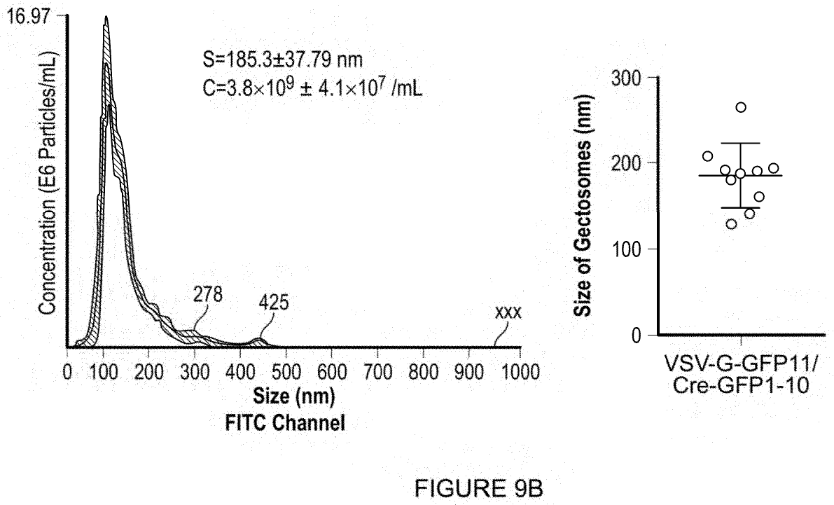

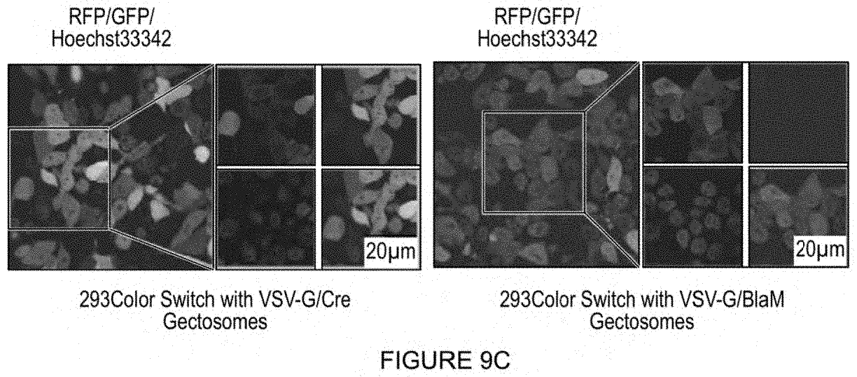

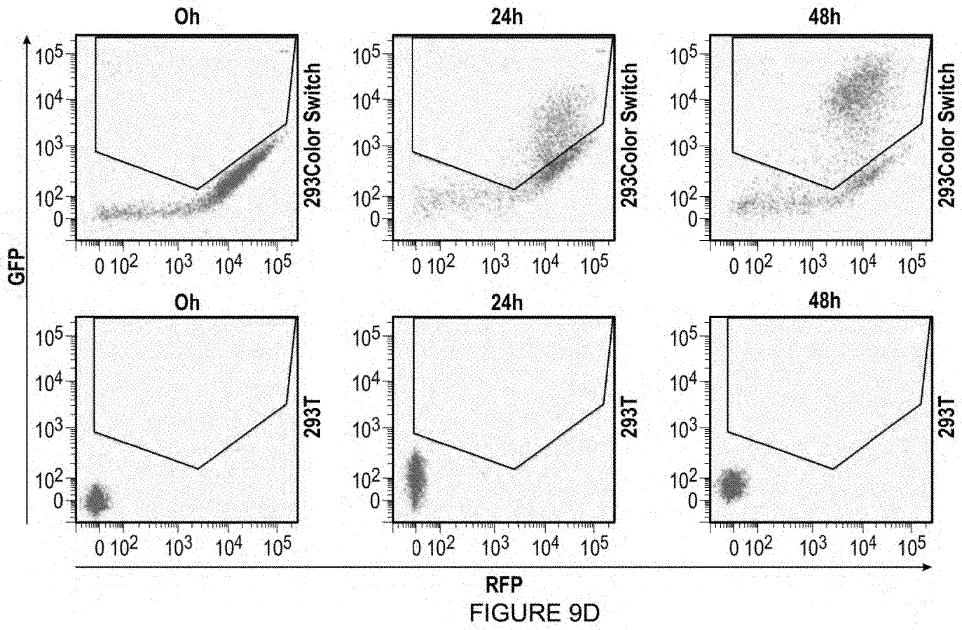

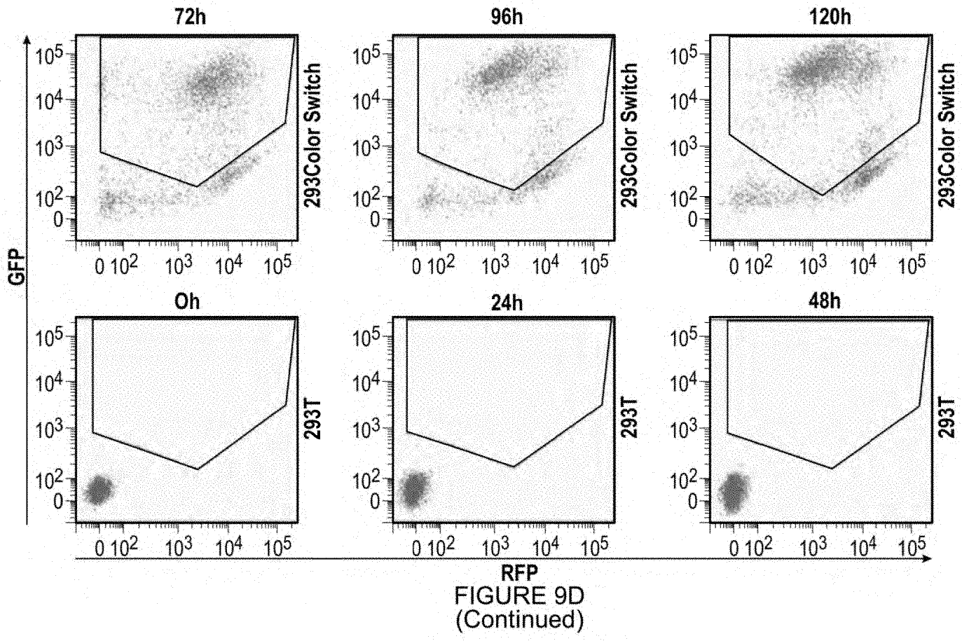

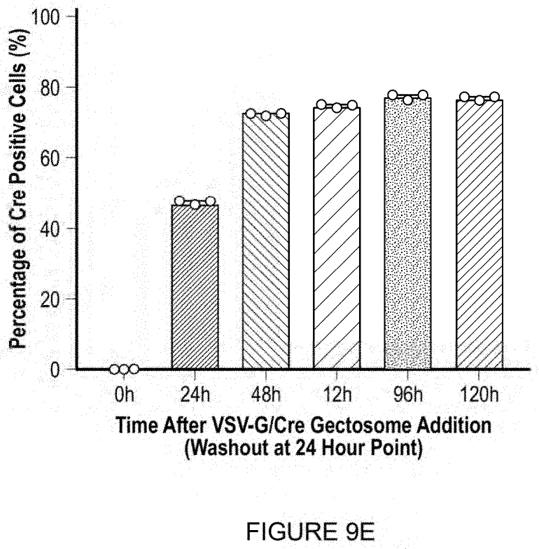

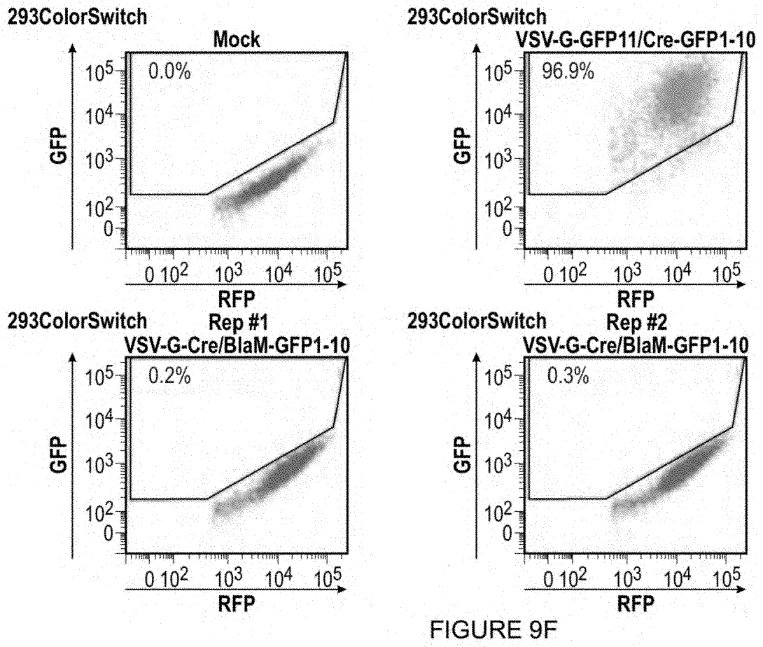

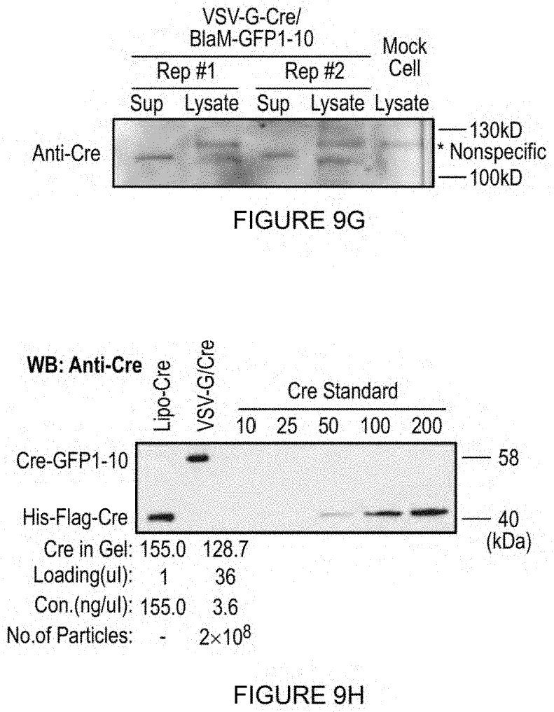

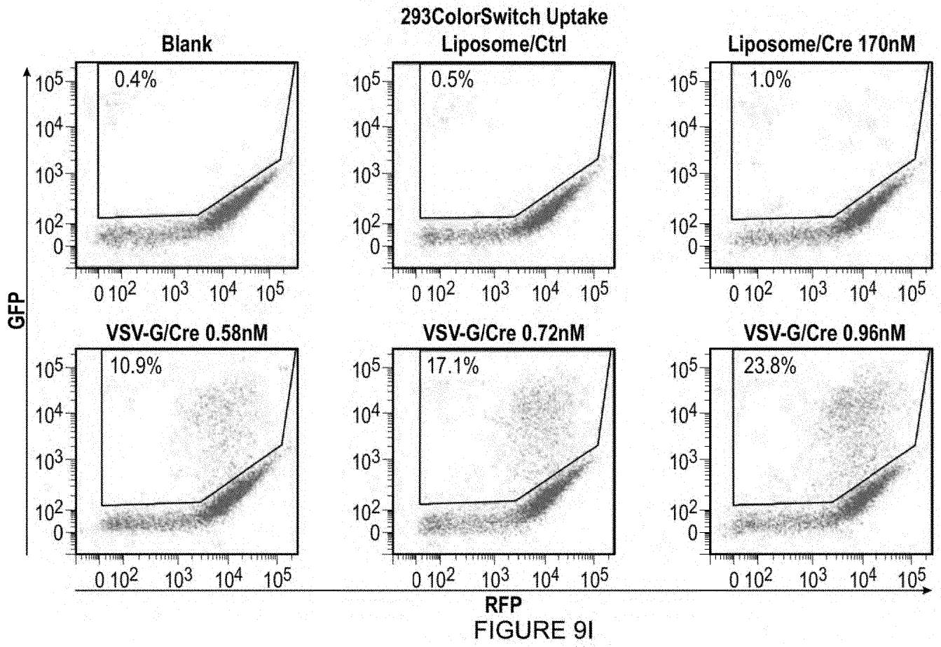

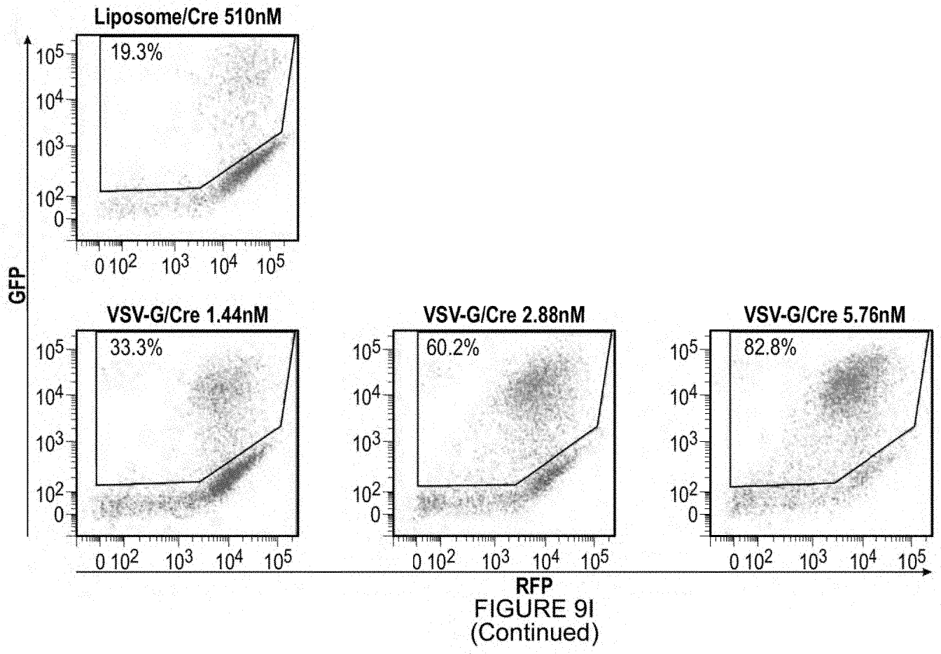

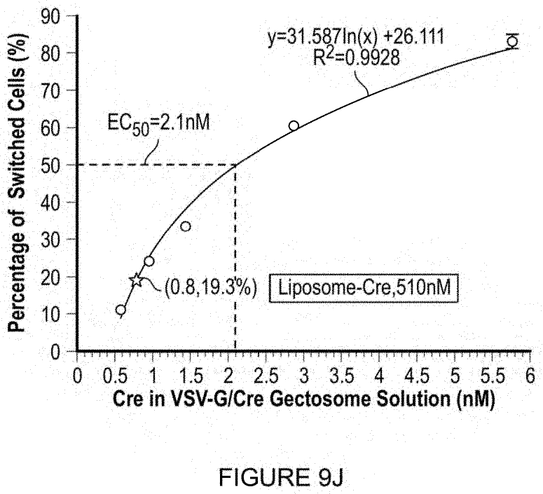

One of the most efficient approaches is the viral delivery of genetically encoded biologics. However, there are safety concerns about persistent exposure of the encoded agent in addition to immunogenic responses, or even oncogenic transformation. As an alternative, liposomal agents have been developed and widely used for delivering nucleic acids from DNA to RNAi. While some liposomal gene therapies have advanced into clinical trials, liposomal delivery of protein is generally less efficient and protein-specific due to the lack of dominant electrostatic property when compared with nucleic acids. The present inventors compared these two methods and showed that liposomes require significantly more Cre protein (630-fold) to achieve the same biological effect (FIGS. 1K and 9H-9J).

Several reasons may contribute to the high delivery efficiency of Cre by Gectosomes of the invention. First, VSV-G mediates efficient cellular entry of VSV particles by endocytosis via binding to the LDL-R family of receptors. Second, as a proficient fusion protein, VSV-G promotes the fusion of viral envelope with the early endosome membrane leading to the release of nucleocapsid into the cytoplasm. Third, due to the presence of chaperone proteins such as Hsp90 and Hsp70 in Gectosomes (Table S2—incorporated herein by reference above), Cre is less likely subjected to denaturation during encapsulation as reported with liposomes in vitro. Therefore, VSV-G enables cargos encapsulated within Gectosomes to overcome the barriers of both the plasma and endosome membranes.

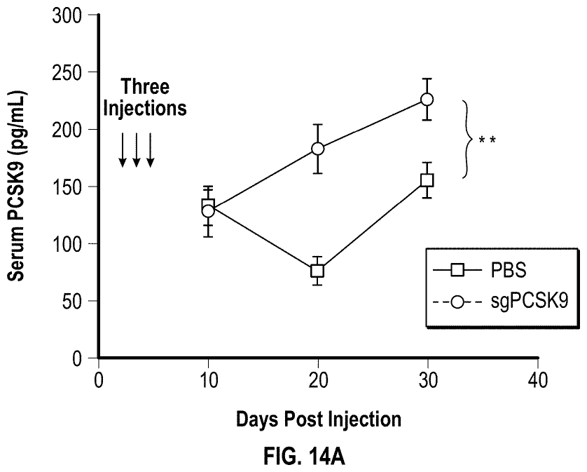

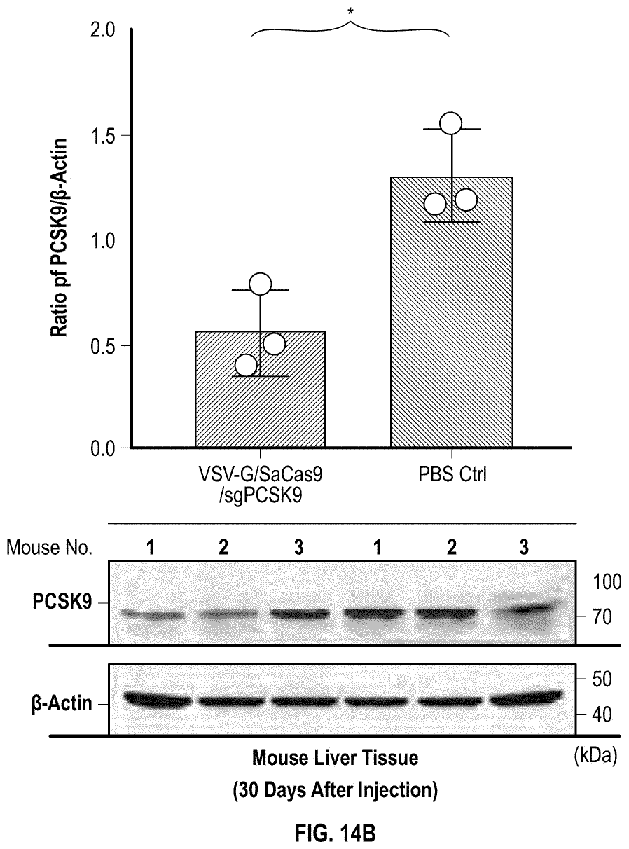

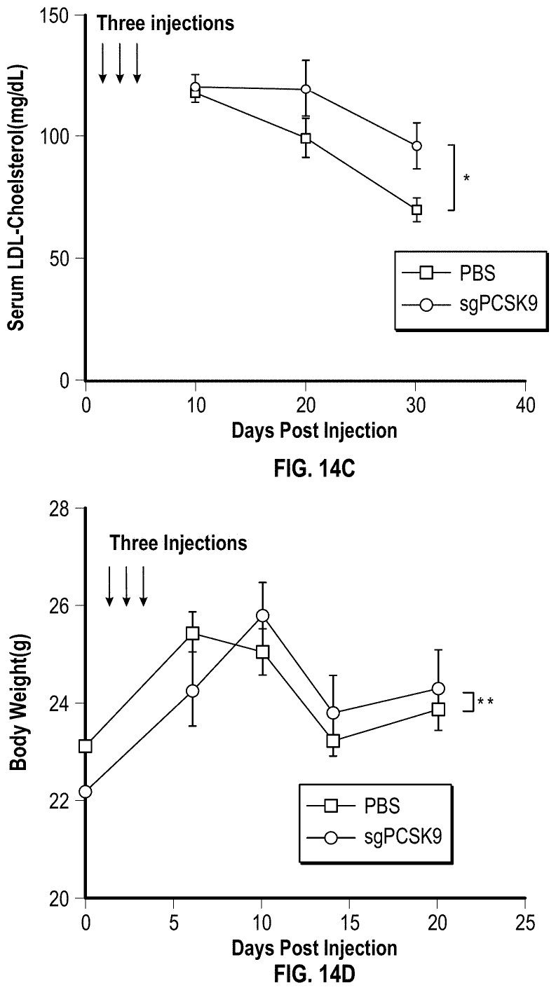

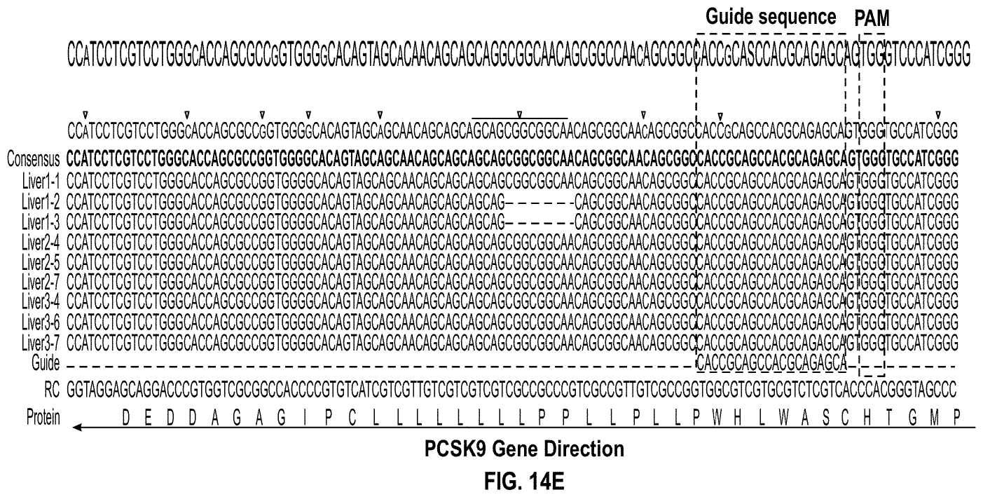

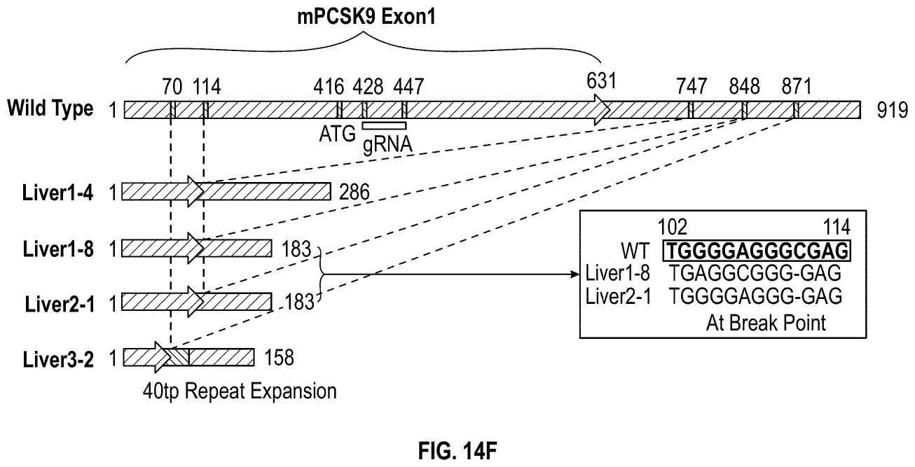

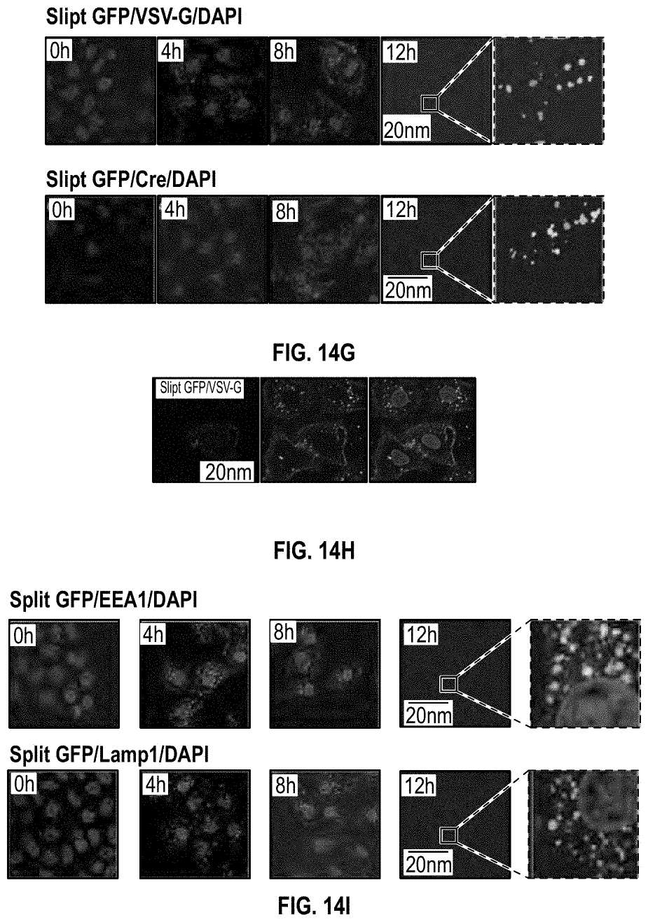

The present inventors performed immunofluorescence microscopy of the time course of VSV-GGFP11/Cre-GFP1-10 uptake in HeLa cells. The signals for VSV-G-GFP11 and Cre-GFP1-10 increase with the time of exposure (FIG. 14G). VSV-G-GFP11 localizes primarily on vesicular structures and largely co-localizes with the reconstituted split GFP signal in vesicles and membrane but not in the nucleus. In contrast, the Cre-GFP1-10 signal is more diffused in the cytosol, membrane, nucleus, and vesicles (FIG. 14G). Co-staining of VSV-G with Cre also shows that a significant fraction of the Cre signals, especially nuclear Cre, do not co-localize with VSV-G (FIG. 14H). Cre-GFP1-10 shows a different time-dependent accumulation pattern fromVSV-G-GFP11 intracellularly, suggesting Cre-GFP1-10 splits from VSV-G-GFP11 in the recipient cells. Gectosomes co-localize significantly with the early endosome marker EEA1 (FIG. 14I), and a small fraction of Gectosome signals co-localize with the late endosome marker Lamp1 (FIGS. 14I and 14J). These results support a model that Gectosomes enter target cells via the endocytic route, and cargo can be released in the target cells. Two pathways for VSV cell entry have been proposed. The prevailing pathway is that VSV-G mediates rapid fusion in early endosomes to release cargo to the cytosol. Another pathway involves VSV fusion with an internal vesicle inside multivesicular bodies (MVB). In the late endosome, the nucleocapsid is released to the cytosol through a back-fusion mechanism using the cellular fusion machinery. The intensive technology suggest that both pathways are possible for Gectosomes delivery, and the second pathway is particularly intriguing in light of the pH-sensitive nature of split GFP complementation

The key difference between the inventive technology and others traditional liposomal delivery methods is the strategy of cargo loading. The invention's approach enables direct tethering of cargo to VSV-G, while others use an artificial membrane protein known as CherryPicker to recruit cargo. The invention showed that the efficiency of cargo transfer with the tethered Cre-GFP1-10 to VSV-G-GFP11 is much higher than with the untethered one (˜26-fold, FIGS. 1H and 1I). The direct fusion of cargo proteins to VSV-G frequently results in low efficiency of cargo delivery since VSV-G with fused cargo may interfere with proper trimer formation required for fusion. Rose and colleagues showed that in functional Vesicular stomatitis virus particles that encapsulate VSV-G GFP, the ratio of wild-type VSV-G to VSV-G-GFP is 4.2:1(Dalton and Rose, 2001). The invention's mass spec data show the ratio of VSV-G-GFP11 to Cre-GFP1-10 is 6:1 in Gectosomes. Since the 16-aa GFP11 tag is less likely to perturb the fusion function of VSV-G, either the trimeric VSVG-GFP11 or a heterocomplex consisting of 2 units of VSV-GGFP11 and 1 unit of VSV-G-GFP11-cargo-GFP1-10 functions well in mediating Gectosome fusion. The two-component Gectosome design with split GFP of the invention takes advantage of this property to enhance more specific cargo encapsulation and reduce nonspecific cellular components.

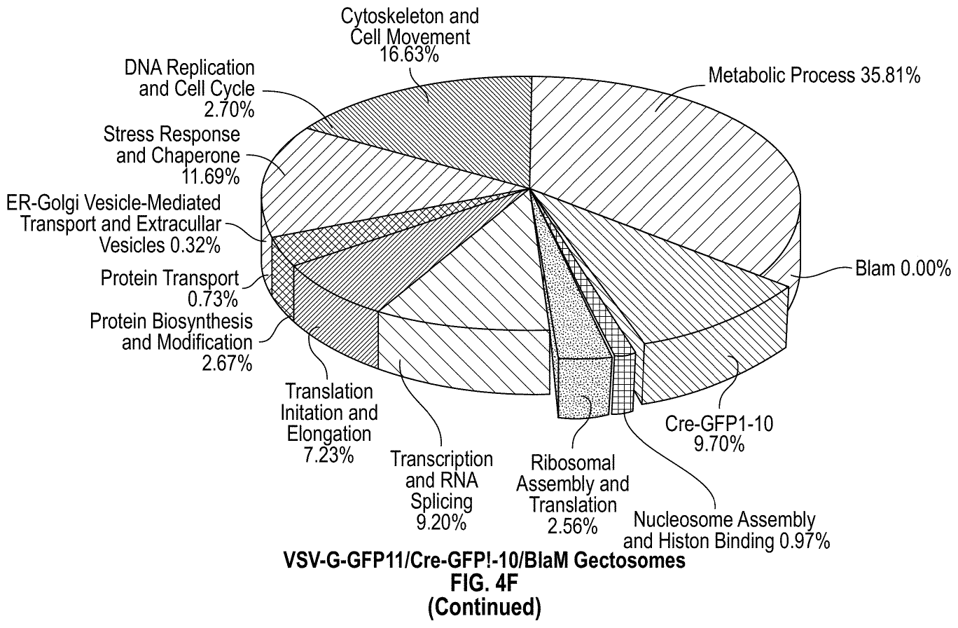

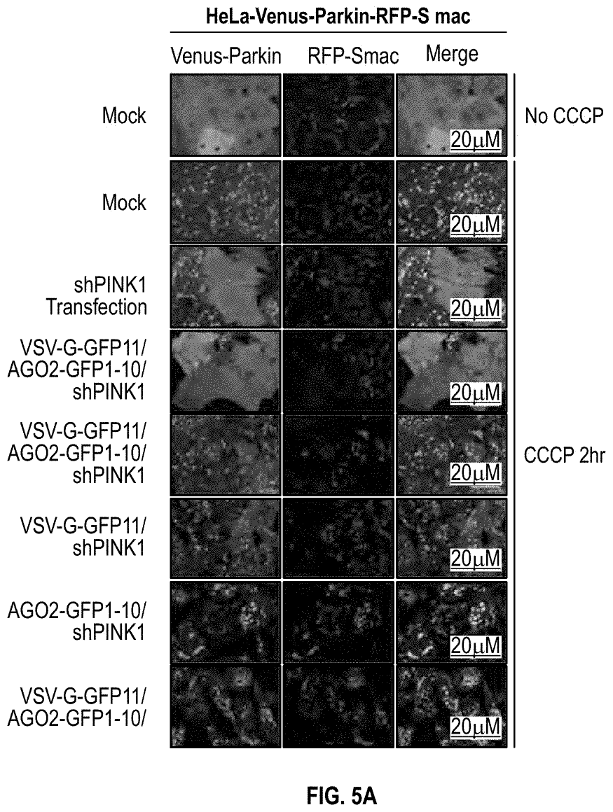

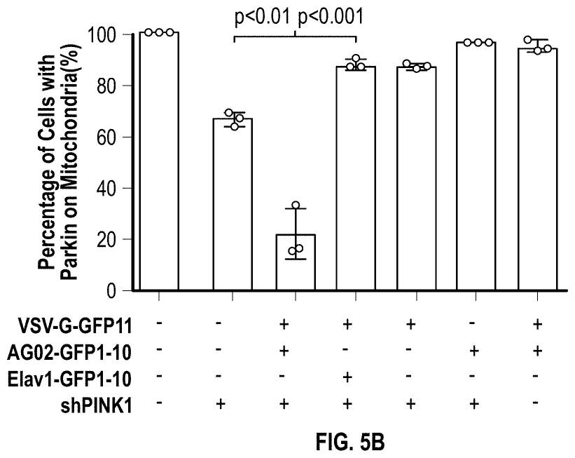

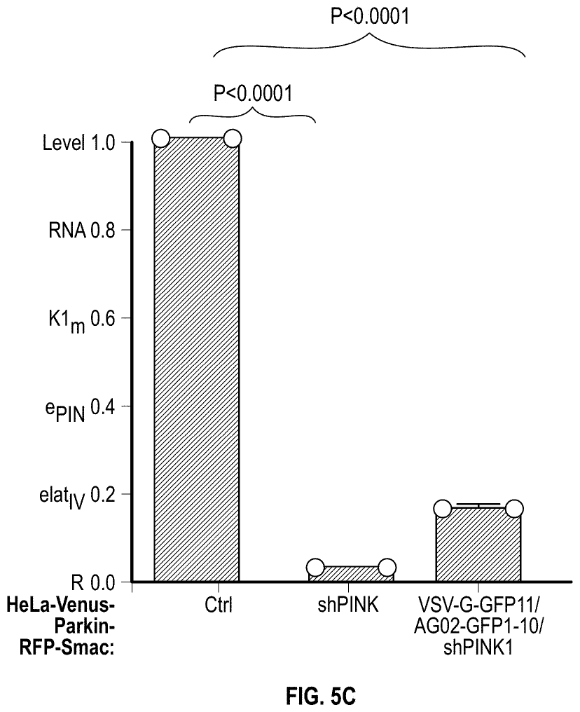

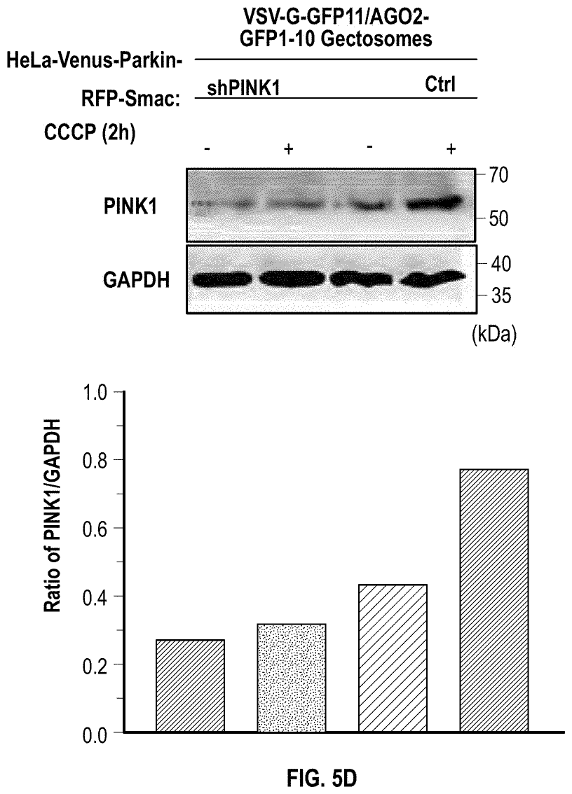

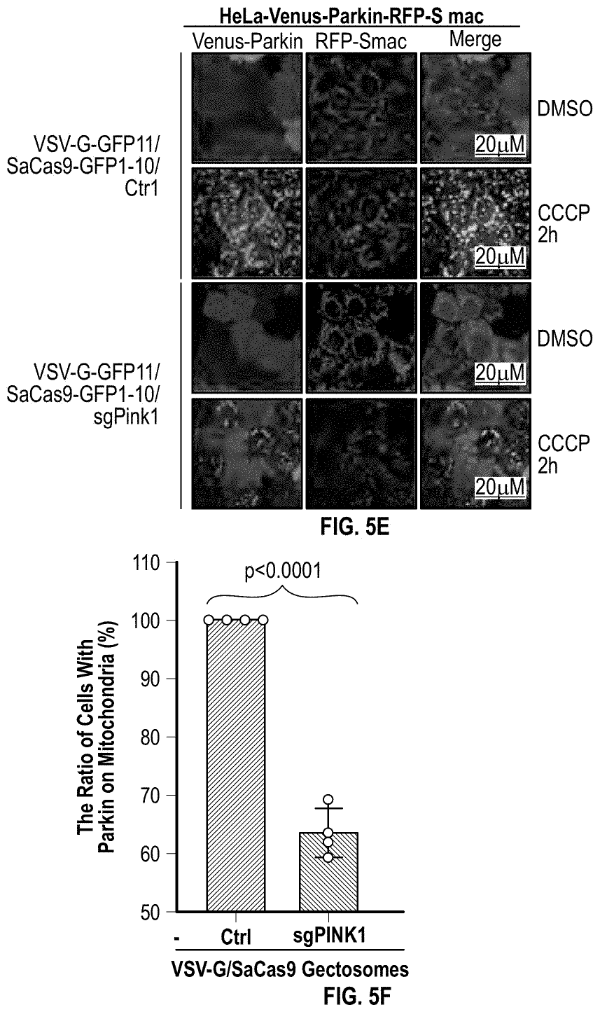

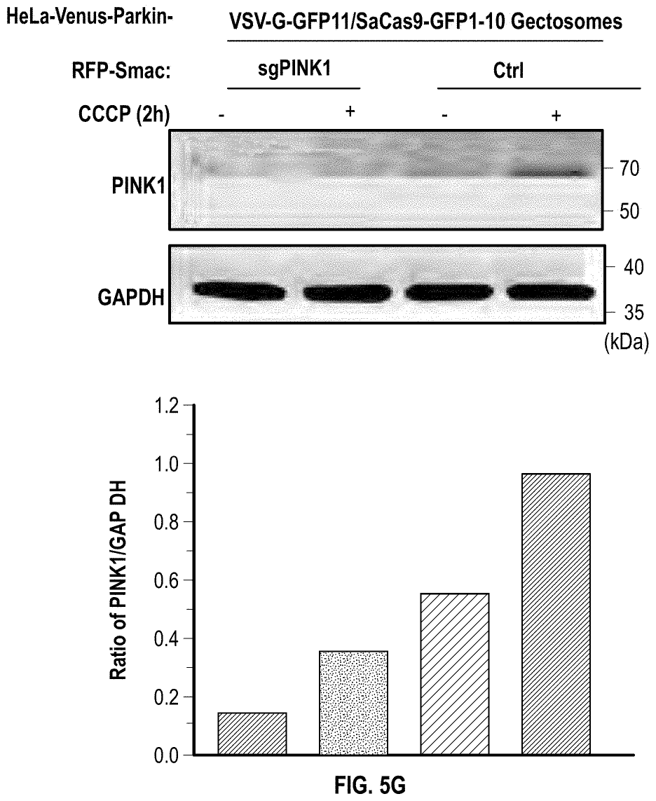

Beyond genome editing, the invention disclosed herein further demonstrated that Gectosomes are more versatile in delivering a variety of biologics. With AGO2, it is possible to perform RNAi with Gectosomes (FIG. 5). Gectosome-based strategies could also be used to transduce other phenotype-modifying agents such as therapeutic antibodies, mRNA, transcription factors, or peptides. Gectosomes can be used to deliver antigens and adjuvants for vaccine development. Since proteins delivered by Gectosomes are dose-dependent, rapidly released, and degraded intracellularly after 24-48 h, at least in the case of BlaM, protein or RNA transduction mediated by Gectosomes is most likely transient in nature and dictated by the intrinsic half-lives of the transduced molecules. This feature is particularly desirable in therapeutic genome editing to minimize potential off-target editing that arises from the persistent expression of Cas9 in the genome.

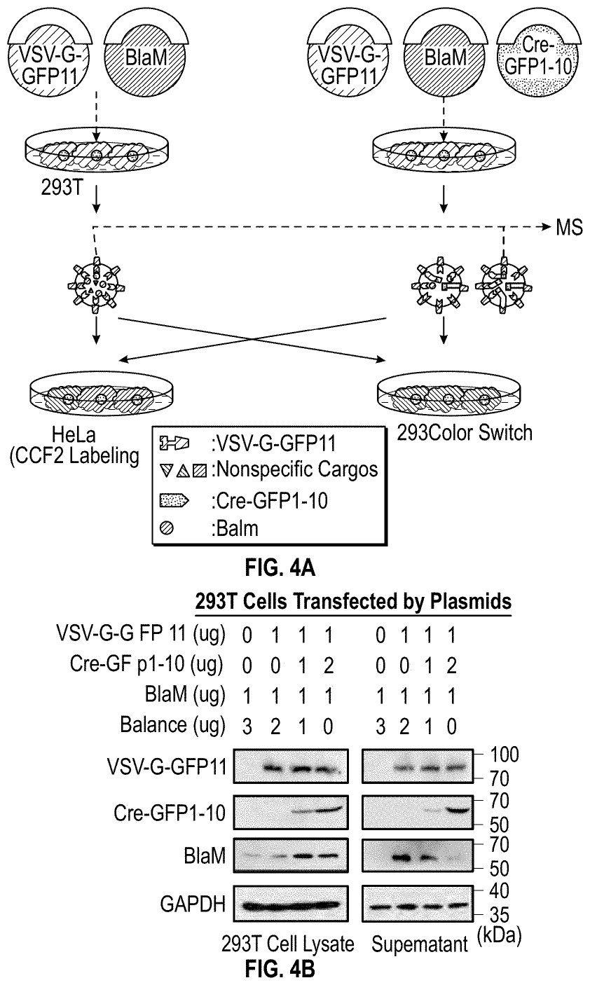

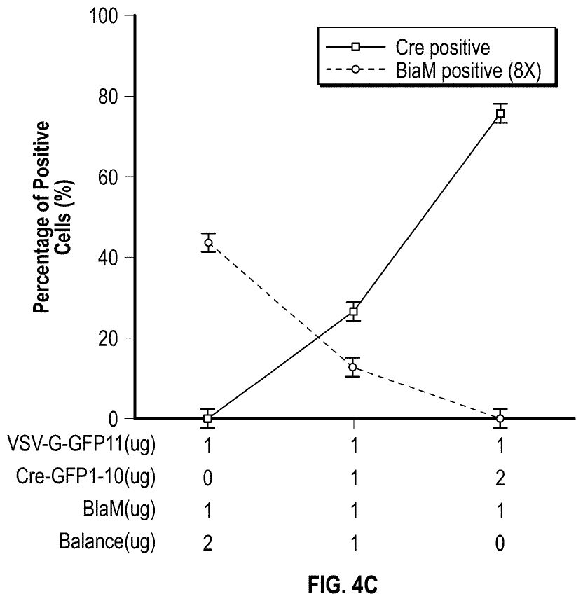

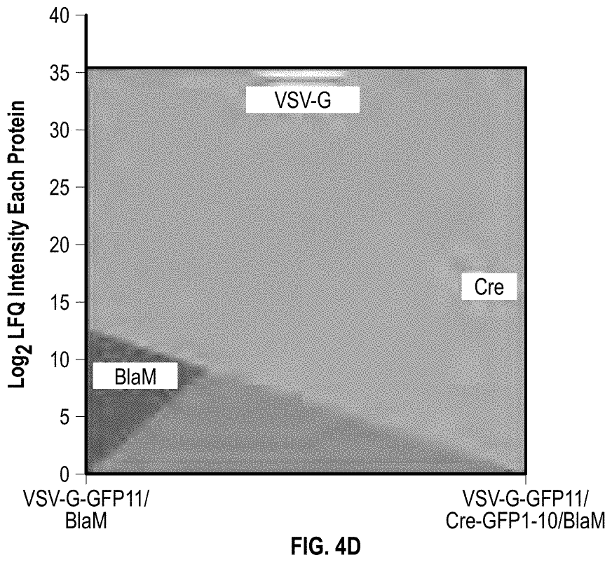

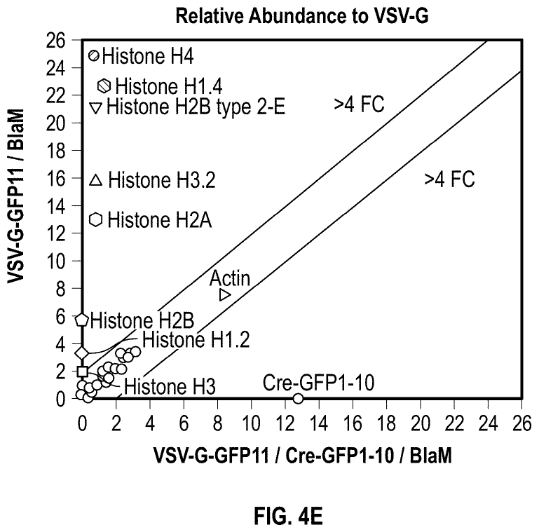

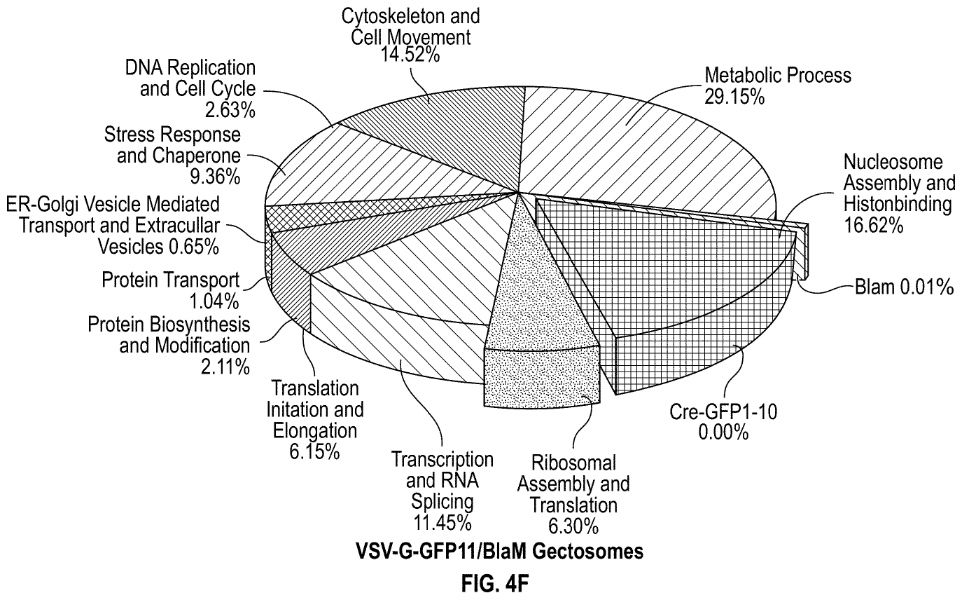

EVs are known to be heterogeneous. A long list of cytosolic and nuclear proteins has been found in VSV-G ectosomes by proteomics analysis. This heterogeneity constitutes a major barrier to developing therapeutics due to a lack of effective strategies to reduce heterogeneity. With an active cargo-loading approach using split GFP, the inventors demonstrated that there is a significant reduction in both the number and the abundance of cellular proteins encapsulated in Gectosomes by quantitative MS analysis (FIG. 4). A surprising result is that histones and nucleic-acid-binding proteins are selectively eliminated from Gectosomes with Cre-GFP1-10. These proteins are frequently found in exosomes and ectosomes. Since histones are highly basic proteins and may bind the inner plasma membrane via electrostatic interactions, it may be that recruitment of Cre-GFP1-10 to the short cytoplasmic tail of VSV-G may occlude histones and other basic proteins from non-specific binding to the negatively charged inner membrane. As an additional effect, passive incorporation of nucleic acids (miRNA, mRNA, and DNA) could also be reduced. Regardless of the mechanisms involved, the active cargo-loading of the invention provides a means to reduce the heterogeneity of EVs and removes a considerable barrier to their development as delivery systems. In summary, the Gectosome approach offers a blueprint for the intracellular delivery of biologics designed to modulate intracellular targets.

### Example 2: Overexpression of VSV-G in Human Cells Elevates Production of VSV-G-Containing EVs

Enveloped viruses often make use of their virus-encoded fusion protein to facilitate membrane fusion with host cells during infection. VSV-G is one of the best studied viral fusion proteins and is frequently used for pseudotyping retroviral or lentiviral particles to enable their entry into a broad range of cell types. During our investigation of the pseudotyping activity of superfolder GFP tagged VSV-G (VSV-G-sfGFP), we noticed that copious amounts of small fluorescence particles ranging in size between 100 and 1,000 nm were present in the culture media from cells transfected solely with VSV-G-sfGFP by flow cytometry analysis (FIG. 1A). To characterize these particles, we transiently transfected 293T cells with sfGFP or VSV-G-sfGFP expression vectors (FIG. 8A) and harvested supernatants for NanoSight tracking analysis (NTA) (FIGS. 1B and 8B). We found that supernatant from VSV-G-sfGFP-transfected cells contained ˜2 3 10⁹ particles per mL with an average size of ˜200 nm in the GFP fluorescence channel. In contrast, only ˜1.6 3 10⁶ particles per mL were detected in the same channel for the control transfection with sfGFP. The total number of EV particles per mL present in the media was comparable based on the particle counts in the clear channel (FIG. 1B). Thus, VSV-G transfection favors the production of fluorescent vesicles by ˜1,000-fold in 293T cells.

To determine whether VSV-G is present on the fluorescent vesicles, we incubated the VSV-G-sfGFP and the control supernatants with magnetic beads coated with a monoclonal antibody (8G5F11) against VSV-G. Strong bead fluorescence was observed with VSV-G-sfGFP supernatant but in neither the control nor beads without antibody, suggesting VSV-G is present on the surface of these particles (FIG. 8C). To examine the morphology of these fluorescent vesicles, we performed transmission electron microscopy (TEM) and VSV-G immunogold labeling studies with the immunopurified vesicles (FIG. 1C). The imaged particles show the expected round-shaped vesicles with an average diameter ˜128 nm, which is slightly smaller than the average size determined by NTA or flow cytometry and could be due to the effects of sample fixation. Immunogold labeling with an anti-VSV-G antibody demonstrates that VSV-G is present on the surface of the vesicle (FIG. 1C). These results show that VSV-G promotes the robust production of EVs enriched with this protein on the surface. Hereafter refer to VSV-G-containing EVs are generally referred to as an embodiment of a “Gectosome” for viral G-protein-containing ectosomes.

### Example 3: Development of Two-Component Fluorescent Gectosomes for Intercellular Transfer of Specific Proteins

Previous work showed that highly concentrated VSV-G EVs could mediate intercellular transfer of VSV-G and a variety of cellular proteins with low selectivity. Passive loading of VSV-G and cargos into vesicles makes these type of EVs highly heterogeneous and low efficiency for delivering specific cargo proteins. We aimed to develop an active loading strategy to recruit specific proteins into Gectosomes and enhance the specificity of cargo delivery. To this end, we used a split GFP system as the building block to construct a two-component Gectosome (FIG. 8D, middle panel). Waldo and colleagues discovered that GFP could be split between the tenth and eleventh b-strands, resulting in separate constructs of a 16-amino acid (aa) fragment (GFP11) and the rest of the protein (GFP1-10). Without the 16-aa peptide, GFP1-10 is nearly non-fluorescent. Upon co-expression of both fragments in cells, GFP11 binds GFP1-10 to reconstitute a functional, fluorescent GFP molecule. To determine whether the split GFP system could be used to bridge VSV-G and its binding partners in cells, we fused VSV-G with GFP11 at its C terminus (VSV-G-GFP11) and a b-lactamase-vpr reporter (BlaM-Vpr) with GFP1-10 at its C terminus (BlaM-Vpr-GFP1-10) (FIG. 1D). 293T cells exhibited higher GFP fluorescence when VSV-GGFP11 and BlaM-Vpr-GFP1-10 were transfected together compared with those that were transfected individually by flow cytometry (FIG. 8A, green versus black traces) or confocal microscopy analyses (FIG. 8E). These results confirm that VSV-G can find its intended cargo protein in cells.

To deliver encapsulated cargos to target cells, two-component Gectosomes need to be efficiently released from the producer 293T cells (FIG. 1D). Supernatant from 293T cells transfected with the split GFP constructs was collected and subjected to NTA. As expected, VSV-G-GFP11/BlaM-Vpr-GFP1-10 particles (VSV-G/BlaM Gectosomes) are fluorescent, and their average size was similar to VSV-G-sfGFP particles (sfGFP Gectosomes), although the yield was slightly lower (33108 particles/mL) (FIGS. 1B and 8B). To confirm the secretion and validate the biochemical composition of two-component Gectosomes, we performed subcellular fractionations followed by western blotting analysis. The result verified that VSV-G and BlaM are released from cells and present in the extracellular vesicle fractions (FIG. 8F). To test whether two-component Gectosomes can transfer encapsulated cargo proteins from producer cells (293T) to target cells (HeLa), we incubated VSV-G/BlaM Gectosomes with HeLa cells. BlaM-Vpr reporter was selected because its enzyme activity can be easily measured by flow cytometry with CCF2-AM, a cell-permeable fluorescence resonance energy transfer (FRET) substrate, which consists of a cephalosporin core linking 7-hydroxycoumarin to fluorescein. BlaM catalyzes the reaction that severs the linkage between the two dyes leading to a loss of FRET so that exciting the coumarin at 409 nm now produces a blue fluorescence signal at 447 nm instead of the FITC signal at 488 nm. As shown in FIGS. 1E and 1F, only supernatant from 293T cells co-transfected with both constructs is capable of delivering BlaM to HeLa or 293T cells (FIGS. 8G and 8H). Moreover, cleavage of CCF2-AM is BlaM specific as Gectosomes produced by co-transfection with Cre-GFP1-10 (see below) have minimal activity (FIGS. 8G and 8H). As a control, we included a VSV-G mutant (P127D) shown to be defective in membrane fusion. This mutant does not affect Gectosomes production or release from the producer cells (FIGS. 1E, 8A, 8E, and 8F). However, BlaM Gectosomes with fusion deficient VSV-G (P127D)-GFP11 fail to mediate the transfer of BlaM-Vpr-GFP1-10 to target cells (FIGS. 1E and 1F). These results suggest fusion is critical for Gectosomes cargo delivery, which is consistent with the role of VSV-G in mediating endosome escape of viral particles.

### Example 4: Robust and Dose-Controlled Intracellular Delivery of Macromolecules by Gectosomes

For exogenous proteins or nucleic acids to reach their intracellular targets, EVs need to fuse with the target cell either at the plasma membrane or inside the endosome following endocytosis. To further evaluate the capability of Gectosome delivery, we tested whether proteins that act on nuclear targets can be transferred from producer cells to target cells. Cre recombinase was selected for these studies since the function of Cre can be readily measured with 293ColorSwitch cells, which stably express a color switch reporter. Upon Cre uptake, cells switch from a strong RFP to a GFP signal due to the excision of a floxed RFP-STOP cassette (FIG. 1G). We fused Cre with GFP1-10 (Cre-GFP1-10) and co-expressed it with VSV-G-GFP11 in 293T cells (FIG. 9A). As with VSV-G/BlaM Gectosomes, the fluorescent Cre Gectosomes were produced massively (˜3.83109 particles/mL). The average size of the Cre Gectosomes is about ˜185 nm in diameter, but the particles appear to be more homogeneous (FIG. 9B) by NTA analysis. To determine whether split GFP enables higher efficiency of Cre delivery, we incubated comparable numbers of Gectosomes from mock, VSV-G-GFP11/Cre-GFP1-10, and VSV-G-sfGFP/Cre-GFP1-10 with 293ColorSwitch cells for 48 h. We found that 66.9% of 293ColorSwitch cells were switched to GFP with Cre Gectosomes, while vesicles from untethered VSV-G-sfGFP/Cre-GFP1-10 resulted in only 2.6% switch, presumably due to passive cargo loading (FIGS. 1H and 1I). The effect of Cre-mediated recombination and the time course of this switching process were confirmed by confocal microscopy and flow cytometry respectively (FIGS. 9C-9E). The same vesicles produced only background fluorescent signals in control 293T cells (FIG. 9D). As a control, we also compared two-component Cre Gectosomes with one-component Gectosomes (i.e., direct VSV-G-Cre fusion, FIGS. 9F and 9G). No significant cell switch was observed with one-component VSV-G-Cre Gectosomes. These results indicate that bioactive Cre delivered by two-component Gectosomes efficiently enters the nucleus to mediate Cre-lox recombination in target cells and that active loading of Cre via split GFP greatly increases (˜26-fold) the efficiency of this process.

Cellular uptake of Gectosomes may require specific interaction between VSV-G and receptor proteins present on the surface of target cells. The specificity and requirements of VSV-G for Gectosome delivery of Cre were investigated with a variant of VSV-G from the vesicular stomatitis virus New Jersey strain (VSV-G-NJ) and a VSV-G antibody (8G5F11) that only binds VSV-G of the Indiana strain, which we used throughout this work. As shown in FIG. 1J, VSV-G-NJ is also capable of efficiently delivering Cre. Conversely, upon incubation with 8G5F11, Cre transfer by VSV-G was completely abolished while VSV-G-NJ mediated transfer was barely affected. These results demonstrate that Gectosome delivery requires VSV-G interaction with the cell membrane and subsequent fusion for efficient cargo release.

How does Gectosomal delivery compare with other dose-controlled delivery systems? Liposomes are well-established vehicles for the delivery of nucleic acids but are known to be not optimal for protein delivery. To compare the delivery efficiency of Gectosomes with liposomes, we first quantified the amount of Cre encapsulated in Gectosomes using recombinant Cre as the standard for immunoblotting (FIG. 9H). Next, we prepared liposomes encapsulating a defined amount of recombinant Cre using previously optimized methods (FIG. 9I). Approximately 510 nM of Cre delivered by liposomes is required to produce 19.3% of cells to switch. With Gectosomes, a similar effect is achieved with about 0.81 nM of Cre (FIGS. 1K, 9I, and 9J). Thus, based on the Cre amount delivered, Gectosomes showed ˜630-fold higher delivery efficiencies than liposomes. These results demonstrate that Gectosomes outperform liposomes in delivering Cre into the nucleus, presumably due to better endosome escape or more robust fusion.

### Example 5: Functional Separation of Gectosomes from Exosomes

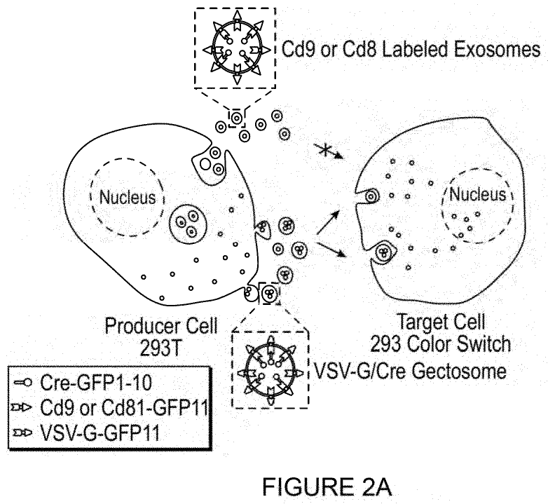

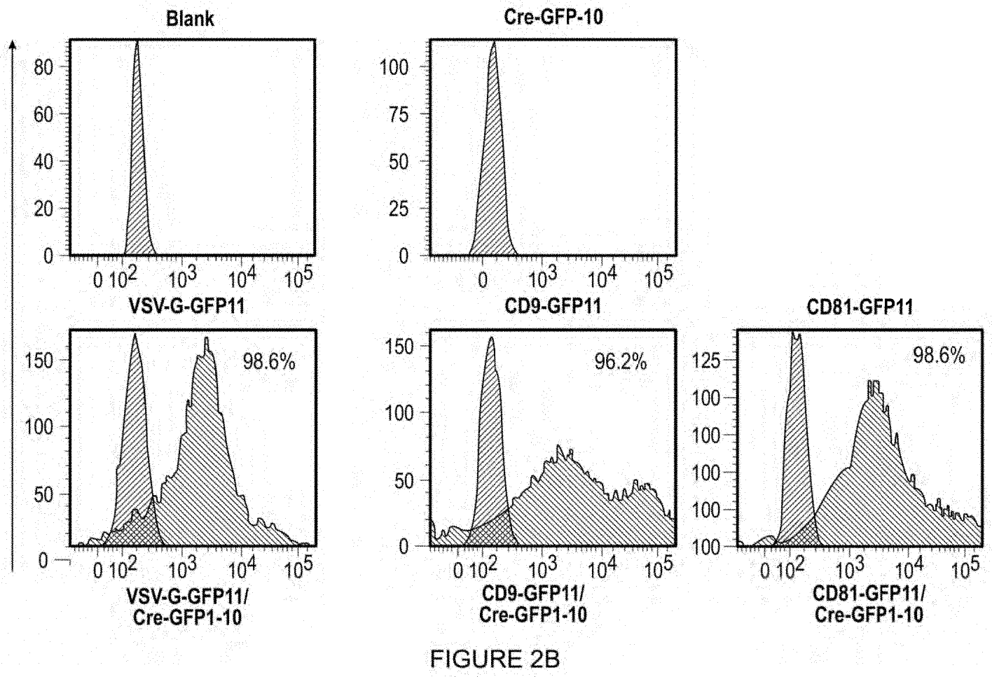

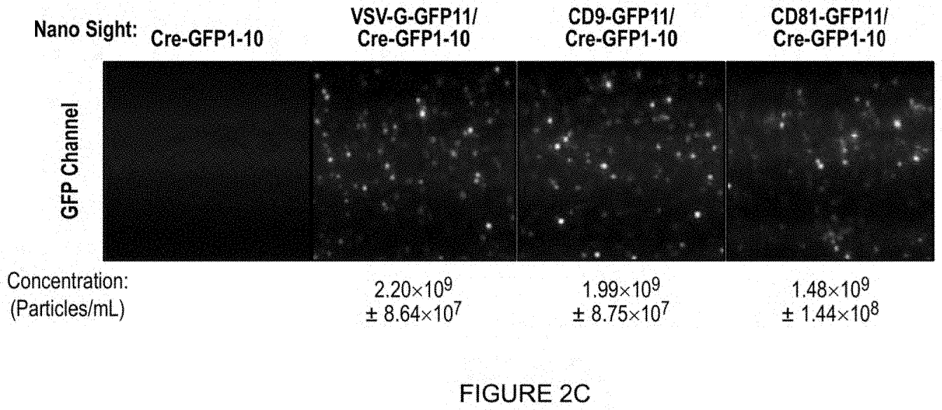

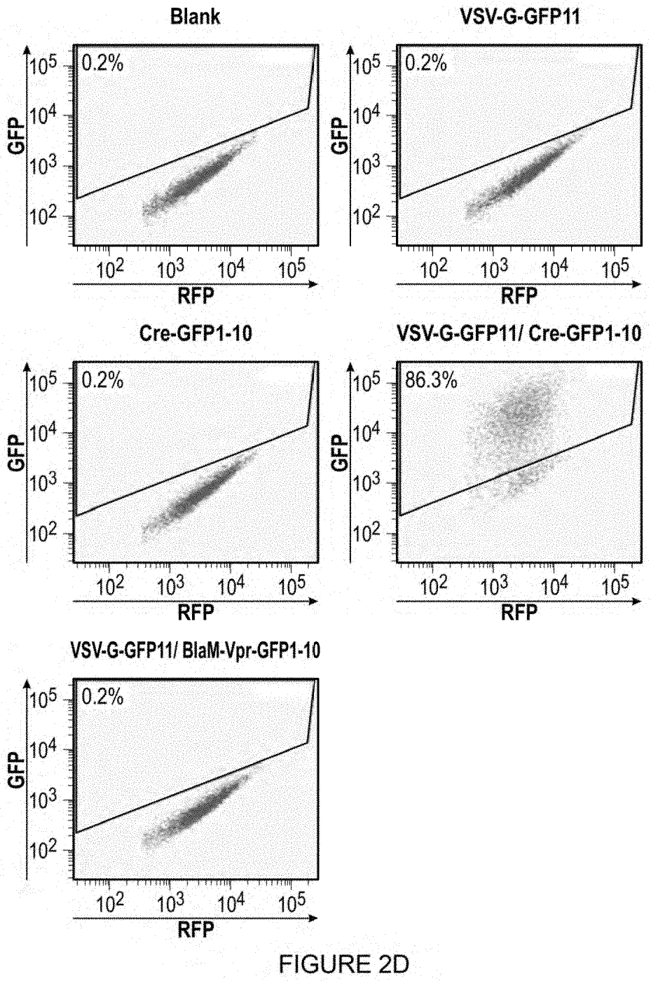

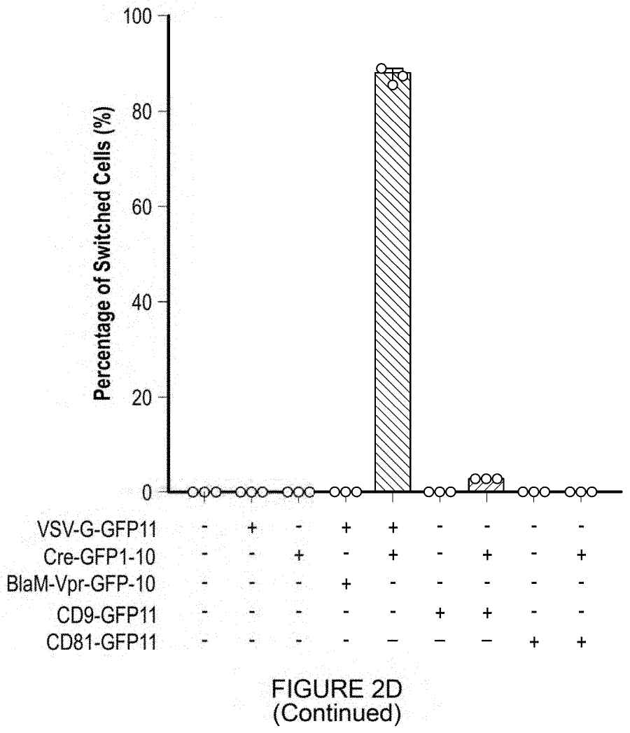

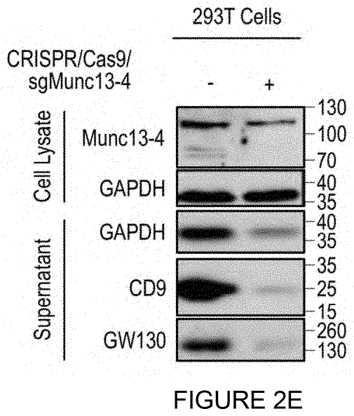

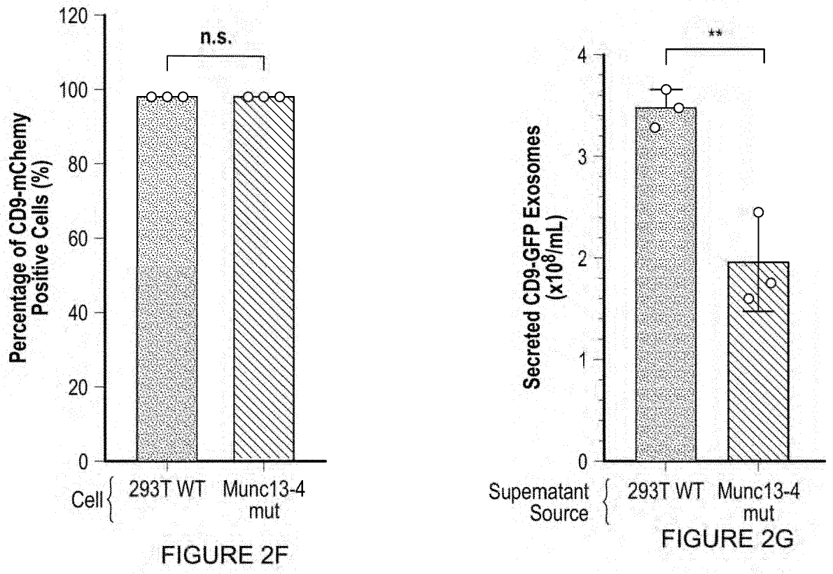

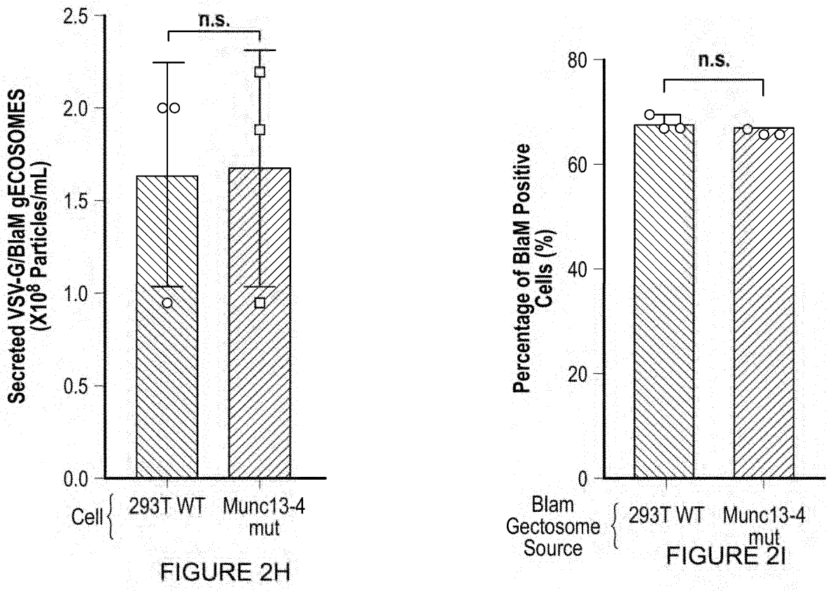

Although exosomes and Gectosomes differ in size and intracellular origin, both can load protein or nucleic acid cargos from producer cells and transfer them to target cells. We wondered whether the active cargo-loading strategy we developed for Gectosomes could be extended to exosomes. To test this notion, we inserted GFP11 to the C-terminal of CD9 and CD81; two protein markers are known to be present on the surface of exosomes (Raposo and Stoorvogel, 2013). Next, we co-expressed CD9-GFP11, CD81-GFP11, or VSV-G-GFP11 together with Cre-GFP1-10 in 293T cells to produce exosomes and Gectosomes (FIG. 2A). Successful and robust reconstitution of the GFP signal was seen with all three pairs (FIG. 2B). NTA of the conditioned media from the transfected cells confirmed that fluorescent EVs were produced at comparable levels (FIG. 2C). While Gectosomes triggered a robust color switch (˜86%) consistent with the transfer of Cre, less than 3% of cells were switched with CD9 EVs, and even lower efficiency of the switch with CD81 EVs was observed (0.4%) when similar numbers of fluorescent particles were applied (FIG. 2D). Although both the loading strategy and production of fluorescent EVs are comparable between types of encapsulation, intracellular delivery efficiency is vastly different. This result suggests that CD9- or CD81-containing vesicles, presumably exosomes, are functionally distinct from Gectosomes for protein transduction.

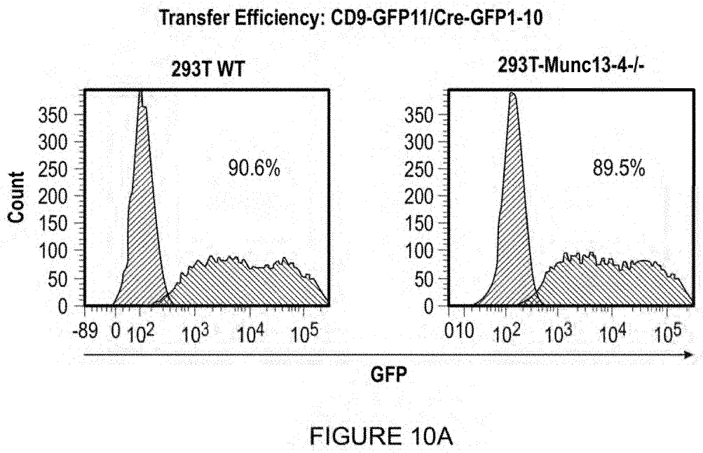

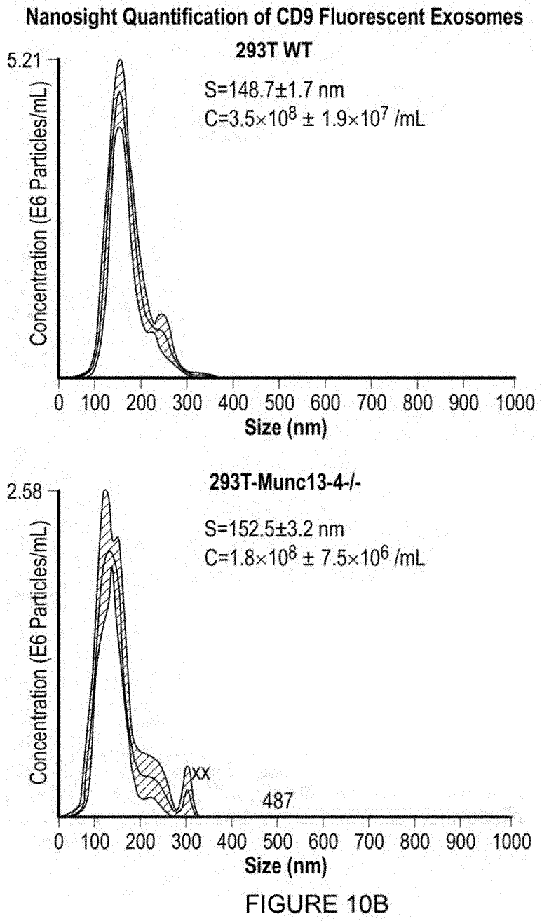

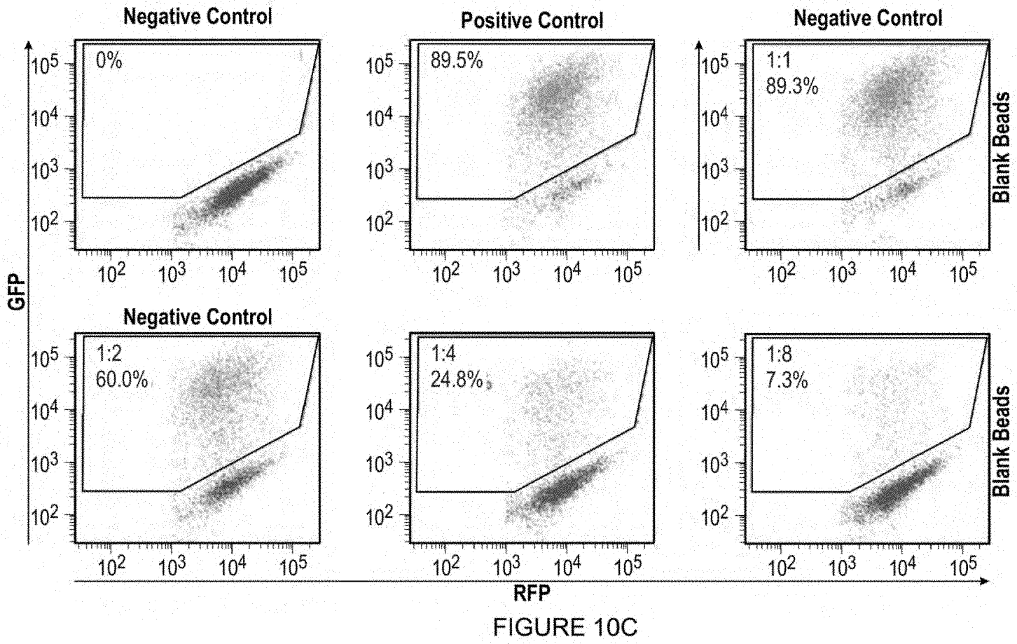

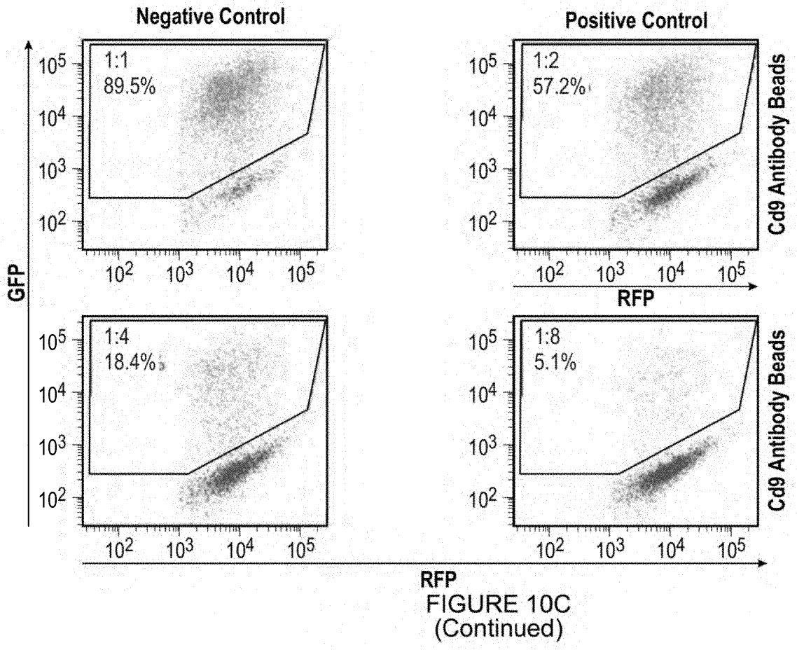

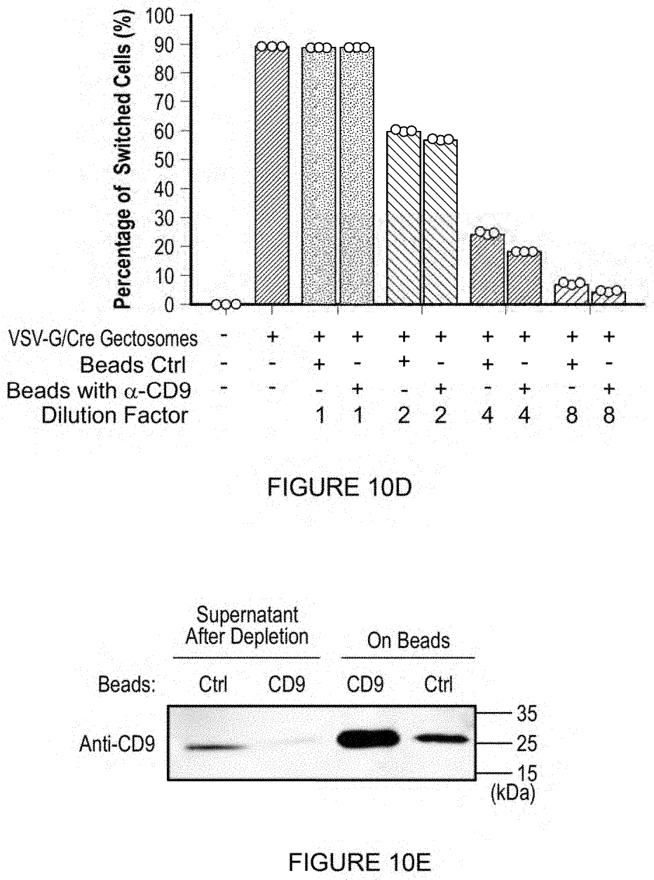

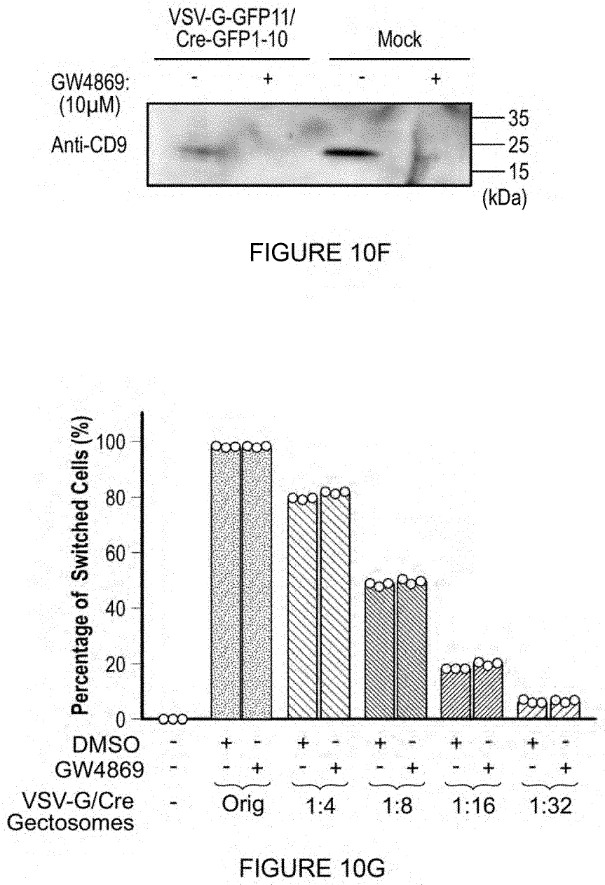

Differentiating and classifying EVs remains a major challenge due to their heterogeneity in size and contents. If Gectosomes and exosomes are separable entities in their biogenesis, we would expect that perturbing the biogenesis of one entity should have minimal effect on the other. Previous studies showed that acute Ca2+ spikes stimulate exosome release in a Munc13-4-dependent manner and that knockdown of this protein significantly inhibited exosome secretion. To test whether Munc13-4 differentially regulates Gectosome and exosome production in 293T cells, we selected stable cell pools expressing Munc13-4 sgRNA and SpCas9 by lentiviral infection. Western blotting showed a partial loss of Munc13-4 expression in the selected cell pool (FIG. 2E). In agreement with previous results, significant reductions in CD9, GW130, and GAPDH levels were observed in supernatant collected from Munc13-4-edited cells (FIG. 2E), consistent with inhibition of exosome release. To determine whether Munc13-4 perturbation also affected the secretion of both endogenous and exogenous CD9, we transiently co-transfected CD9-GFP11 and Cre-GFP1-10 into wildtype and Munc13-4-edited cells (FIG. 10A). The expression of CD9-GFP11/Cre-GFP1-10 in wild-type cells was indistinguishable from expression in edited cells (FIGS. 2F and 10A). However, the number of GFP-positive CD9 exosomes from Munc13-4 edited cells was ˜2-fold lower than from wild-type cells, as measured by FACS (FIGS. 2G and 10B). Thus, the depletion of Munc13-4 causes intrinsic defects in the production of CD9-positive exosomes. To investigate Gectosome production, we transfected wild-type and Munc13-4-edited cells with VSV-GGFP11 and BlaM-Vpr-GFP1-10. We found that fluorescent Gectosome secretion is comparable between these two cell lines (FIG. 2H), and the efficiency of BlaM delivery to HeLa cells is also indistinguishable between the Gectosomes collected from those two producer cell lines, indicating that Gectosome production is independent of Munc13-4 (FIG. 2I).

We further investigated whether Gectosomes can be separated from exosomes biochemically. Using magnetic beads coated with an anti-CD9 antibody, we depleted CD9 exosomes from VSV-G-GFP11/Cre-GFP1-10 Gectosomes and measured the remaining Gectosome activity following serial dilutions (FIGS. 10C-10E). Only a small reduction in Gectosome activity was observed (FIGS. 10C and 10D), suggesting that the bulk of Gectosome activity is not significantly affected by the removal of CD9 exosomes.

Finally, we also tested the effect of GW4869, a potent neutral sphingomyelinases inhibitor known to block exosome biogenesis, on Gectosome production in the producer cells. Following mock and co-transfection of VSV-GGFP11/Cre-GFP1-10, 293T cells were treated with GW4869 (10 mM) to assess the effect on CD9 production and Gectosome activity. As expected, GW4869 reduced CD9 exosome secretion in both mock and Gectosome-producing cells (FIG. 10F), whereas the activity of VSV-G-GFP11/Cre-GFP1-10 was not affected (FIG. 10G). This result further supports that Gectosomes and exosomes are functionally separable.

### Example 6: Purification, Quantitation, and Mathematical Modeling of Gectosomes

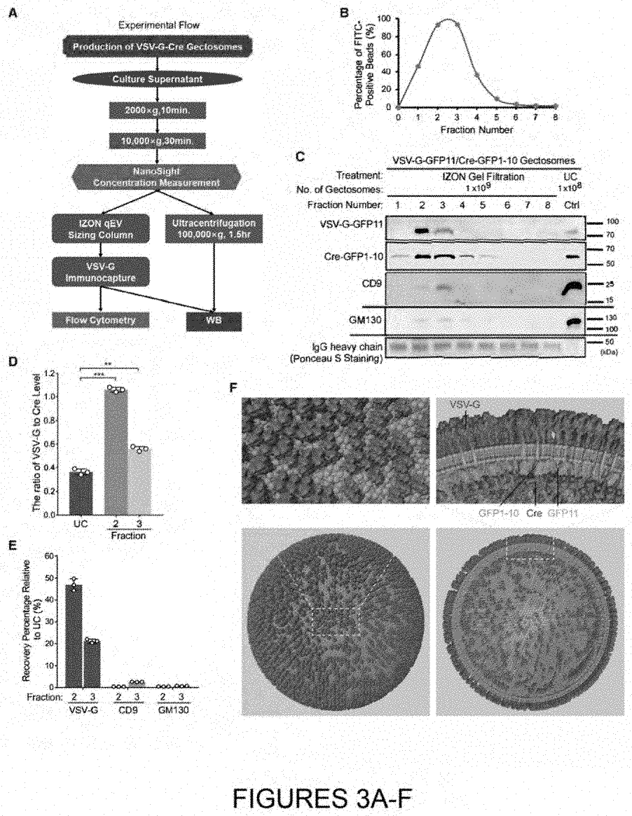

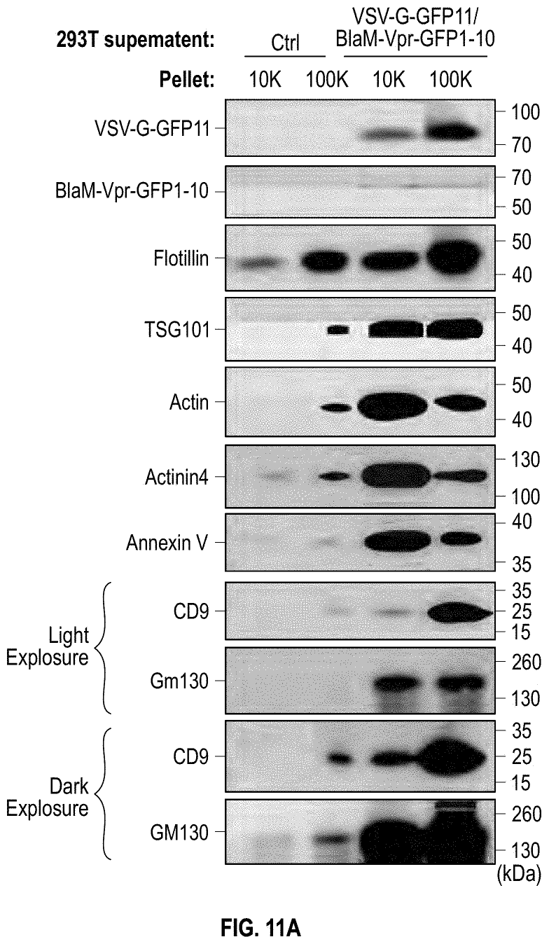

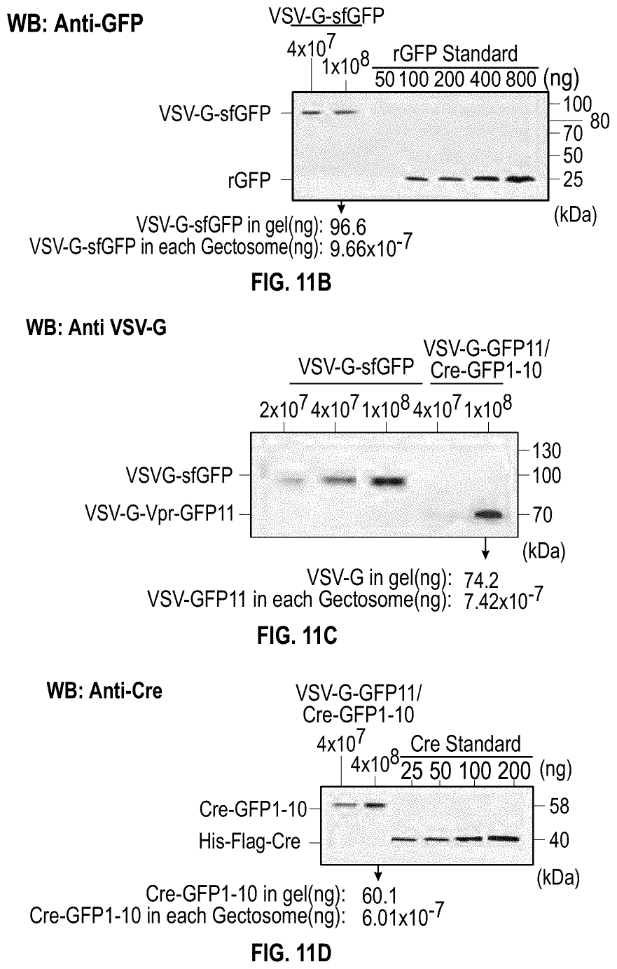

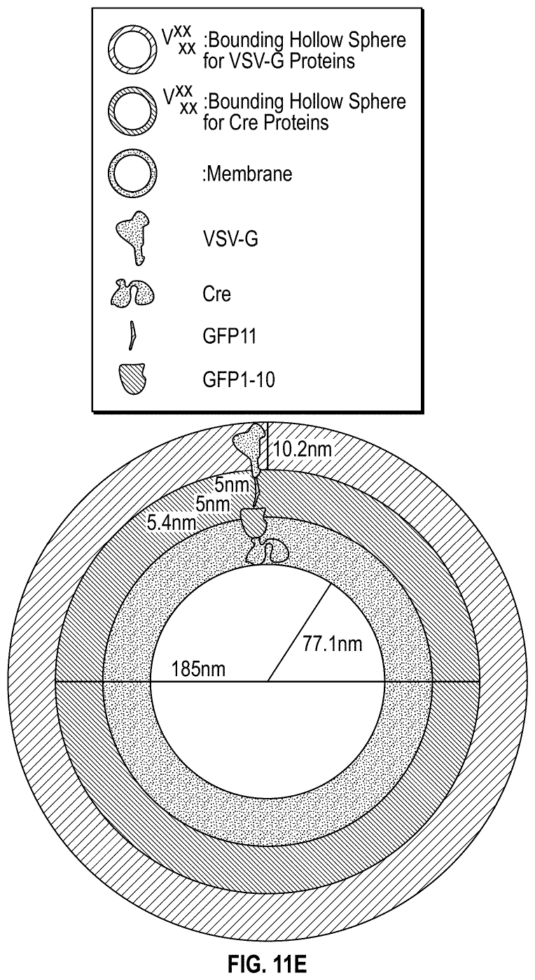

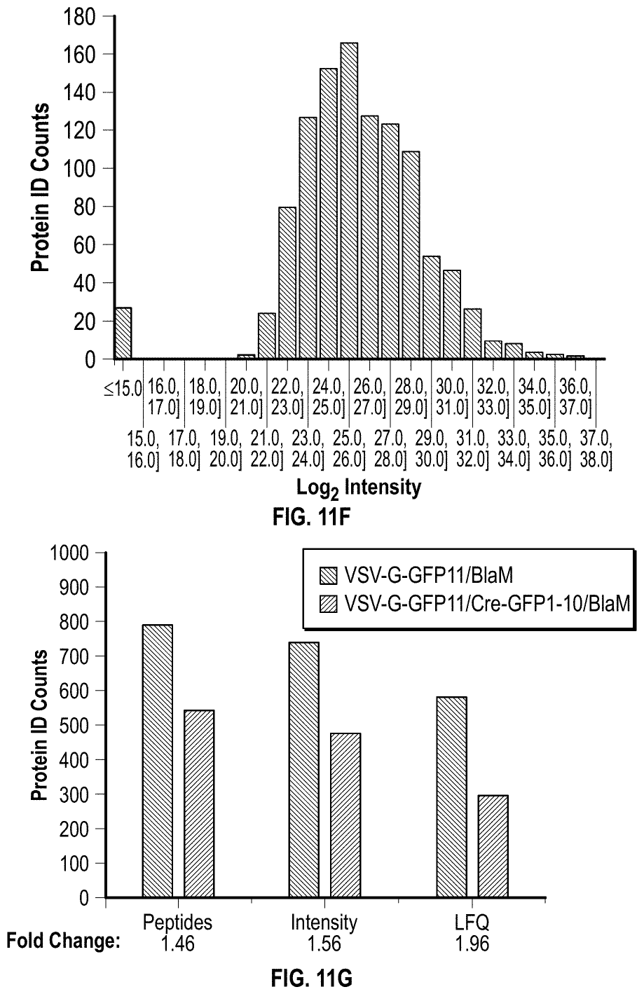

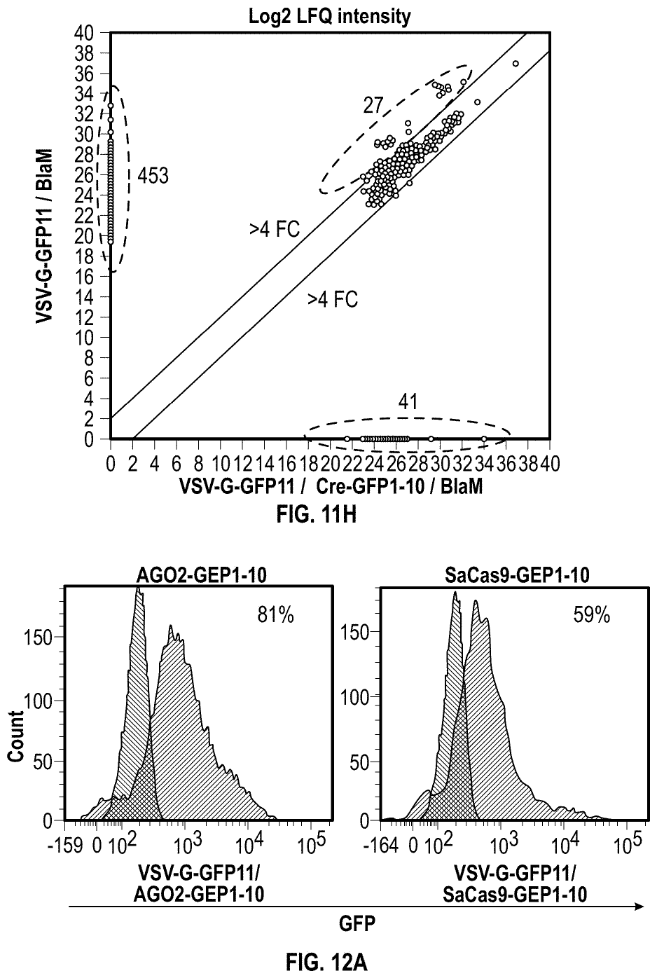

Using a well-established protocol developed for purification of exosomes, we tried to separate Gectosomes from other EVs by differential ultracentrifugation (UC) and flotation in a density gradient. While this procedure effectively enriches exosomes as measured by CD9 and GM130, Gectosomes components are also enriched in 100-K UC sediments (FIG. 11A). To solve this problem, we developed a scalable purification protocol for Gectosomes that involves four major steps (FIG. 3A). After two differential centrifugation steps, we applied the resuspended 10K pellet on a qEV 70 nm (IZON) size SEC column and collected the fractions with FITC fluorescence. Magnetic beads with immobilized anti-VSV-G 8G5F11 antibody were used to capture the Gectosomes before final elution with low pH glycine. Shown in FIGS. 3B and 3C, most Gectosomes are present in the second and third qEV fractionations based on the fluorescence intensity and captured VSV-G-GFP11 and Cre-GFP1-10 by western blot. VSV-G versus Cre ratios in fraction ⅔ are increased dramatically compared with that of the UC sample (FIGS. 3C and 3D). While 100,000 3 g ultracentrifugation fractionation results in highly enriched CD9 and GM130 (FIGS. 11A and 3C), IZON fractionation followed by immunocapture significantly decreases CD9 and GM130 but increases VSV-G in fractions 2 and 3 when compared with the corresponding proteins in UC samples (FIGS. 3C and 3E). This result suggests that our purification protocol is very effective in removing exosomes, while the residual amount of CD9 in the Gectosome fractions may come from the cell surface. This result also further supports a biochemical distinction between Gectosomes and exosomes.

To develop a 3D molecular model for a prototypic Gectosome, we first performed quantitative measurements of VSV-G and Cre in Gectosomes by immunoblotting using purified recombinant proteins as standards. The number of VSV-G-GFP11 and Cre-GFP1-10 is estimated at ˜5,620 and ˜933 molecules respectively per gectosome (FIGS. 11B-11D). We constructed a 3D molecular model of a Gectosome filled with VSV-G, sfGFP, and Cre from their known molecular structures in PDB using 3D software Blender (https://www.blender.org) (FIGS. 11E and 3F). The model derives from the “best guess” numbers (Table S1 and STAR Methods) and needs to be refined when better analytical techniques become available. Nevertheless, an average gectosome model can guide our understanding of Gectosomes. In this model, Cre-GFP1-10 occupies about ˜13% of the total lumen space and spatially fills ˜41% the hollow sphere beneath the inner membrane due to its strong association with VSV-G-GFP11 (Kd<1 nM) (FIG. 11E and STAR Methods). The modeling results prompted us to investigate what other proteins may be present in Gectosomes and whether active loading of Cre-GFP1-10 deters the recruitment of certain cellular proteins.

### Example 7: Active Loading of VSV-G Gectosomes with the Split GFP System Reduces Nonspecific Incorporation of Cellular Proteins

We considered two possible models of cargo encapsulation in Gectosomes with the split GFP system. One model is that active loading of Cre-GFP1-10 simply adds to the repertoire of existing proteins in Gectosomes without changing its baseline composition. A second competing model is the encapsulation of Cre-GFP1-10 remodels Gectosomes by specifically outcompeting other cellular proteins. The second model predicts that an increasing amount of Cre-GFP1-10 in Gectosomes will reduce non-specific incorporation of a cytosolic reporter protein. To test this model, we transfected 293T cells with the same amount of VSV-G-GFP11 while varying the ratio of input Cre-GFP1-10 (specific protein) to BlaM (non-specific protein). Gectosomes were collected and incubated with either 293ColorSwitch or HeLa cells to measure the activity of these two enzymes, respectively (FIG. 4A). Supernatants and cell lysates were also blotted with relevant antibodies to verify the amount of Cre and BlaM in Gectosomes (FIG. 4B). The increasing Cre-GFP1-10 lowers non-tethered BlaM in Gectosomes, which is corroborated by the activity of Cre and BlaM measured in two cell lines described above (FIG. 4C).

To directly test the second model, we purified Gectosomes produced by co-transfection with VSV-G-GFP11 along with untethered BlaM with or without specific cargo protein Cre-GFP1-10 i.e., VSV-G-GFP11/BlaM (VB) versus VSV-G-GFP11/Cre-GFP1-10/BlaM (VCB) (FIG. 4A). Since Gectosomes encapsulate untethered BlaM protein, we reasoned that it could serve as a proxy for non-specific recruitment. Harvested Gectosomes were immunocaptured with anti-VSV-G agarose beads and subsequently eluted off the beads with 1% SDS buffer. An equal amount of input proteins in duplicates were digested with trypsin and analyzed by label-free quantification (LFQ) mass spectrometry (MS). The values of Log2 LFQ intensity of all detected proteins are ranged from around 21 to 35, which means the data quality is suitable for further analysis (FIG. 11F; Table S2 which can be found at and is specifically incorporated herein by reference). We first compared the Log2 LFQ intensity values of the known proteins (VSV-G and BlaM) shared in these two Gectosome samples (FIG. 4D; Table S2). The number of VSV-G peptides detected (27 versus 26) and the Log2 LFQ intensity value (35.58 versus 35.95) are comparable, suggesting the equivalent amounts of VSV-G-GFP11 in two Gectosomes. Three peptides of BlaM (Log2 LFQ intensity is 12.6) were detected in VB Gectosomes, in contrast, none was detected in VCB Gectosomes. This result suggests that BlaM is excluded in the presence of Cre-GFP1-10, which supports the results of the experiment shown in FIGS. 4B and 4C.

Next, we quantified the endogenous proteins identified in both Gectosome samples (Table S2). MS peptide, intensity, and LFQ data show that VCB Gectosomes carry less randomly packaged endogenous proteins (FIG. 11G). We plotted the Log2 LFQ intensity values of all detected proteins in both samples (FIG. 11H). There are about 323 proteins shared between the two samples (FIG. 11H). Of these, 27 proteins appear to be more abundant in the VB sample. Additionally, 453 proteins were found only in the VB Gectosomes and 41 proteins were found only in the VCB Gectosomes. Overall, VB Gectosomes contain more diverse set of proteins than VCB Gectosomes do, even though both species contain an almost identical amount of VSV-G-GFP11.

The MS data were further analyzed in the context of our 3D-molecular model. First, we ranked proteins identified in two Gectosomes by their abundance relative to VSV-G-GFP11 based on the LFQ intensity. The result showed a dramatic reduction of histones in the presence of Cre-GFP1-10 (FIG. 4E). Second, we estimated the molecular volume (partial specific volume) of the lumen proteins from their molecular weight using a simplified biophysical equation (Erickson, 2009). This approximation allowed us to compute the packing of Gectosomes in response to active loading. We summed up the total volume of lumen proteins in each Gectosome scaled to their relative abundance to VSV-G-GFP11. The total calculated volume of these proteins account for ˜20% of our 3D molecular model's theoretical lumen volume for VB and VCB Gectosomes (Table 10). Plotting the proteins' volume distribution according to their GO classifications shows that major differences between VCB and VB Gectosome's cargo composition are the nucleosomal and ribosomal proteins (FIG. 4F), which agrees with the abundance changes described above (FIG. 4E). Taken together, these results support a model that active Gectosome loading via split GFP outcompetes non-specific encapsulation of cellular proteins and thereby reduces the heterogeneity of Gectosomes.

### Example 8: Gectosomes can Deliver Versatile Cargos into Target Cells and Program Gene Expression

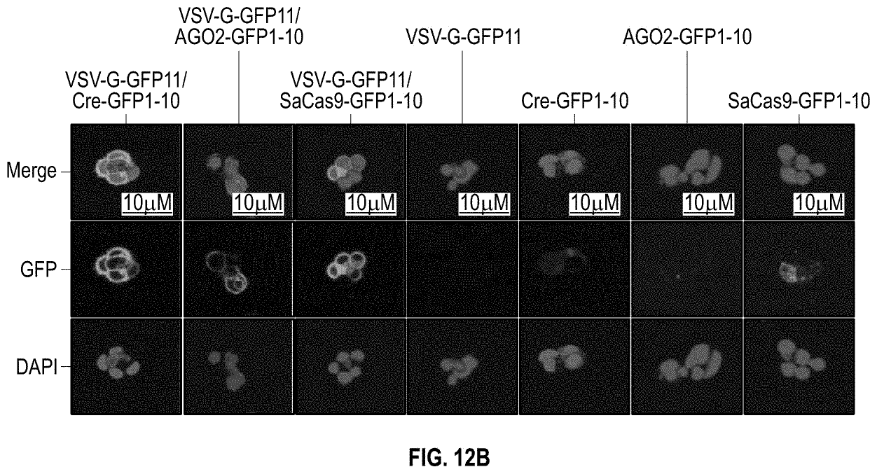

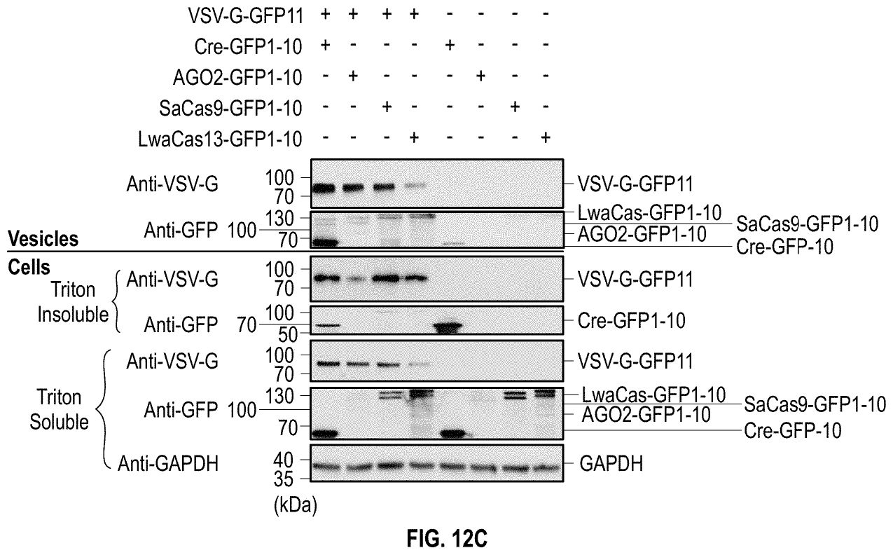

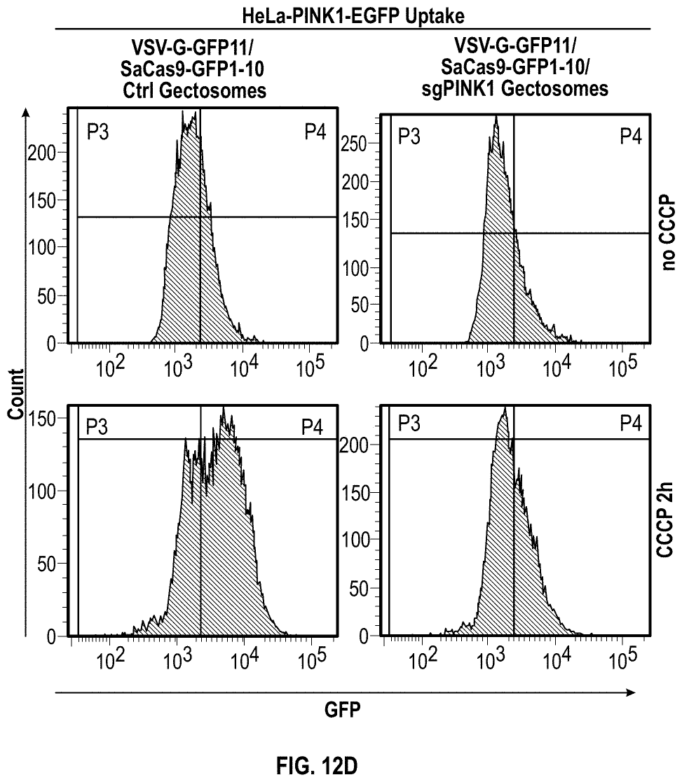

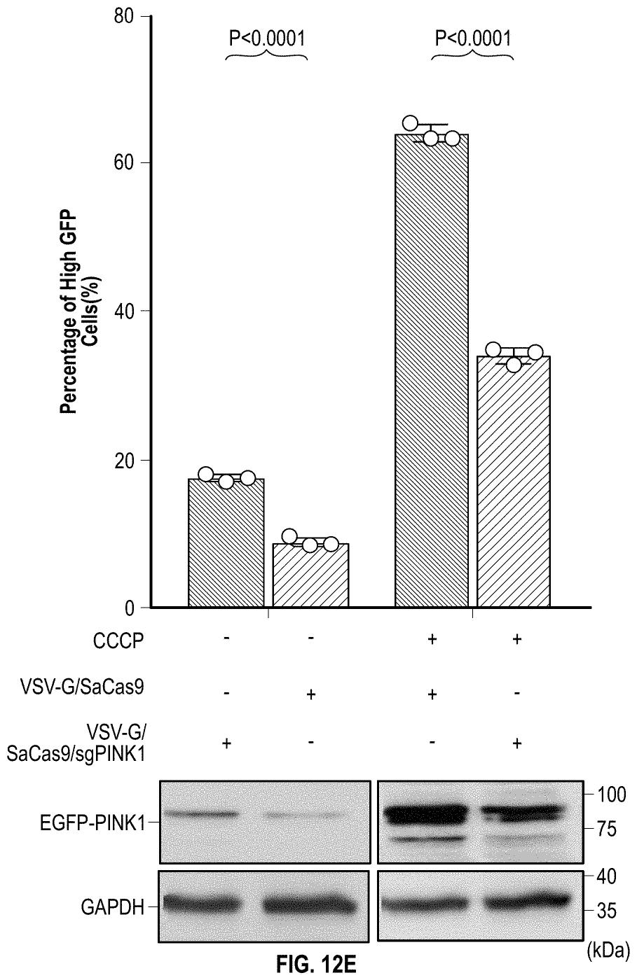

Can Gectosomes be engineered to deliver gene modifying functionality to target cells? To address this question, we designed Gectosomes that encapsulate AGO2 and SaCas9. Flow cytometry, confocal microscopy, and western blot analyses confirmed that these proteins formed complexes with VSV-G-GFP11, mediated by split GFP, and were released from cells (FIGS. 12A-12C). To test whether Gectosomes can package and deliver RNA-interfering functionality to target cells, we used a co-transfection system where cells received Gectosomes containing GFP1-10 fused to AGO2 and the shRNA targeting the mitochondrial kinase PINK1. AGO2 is a component of the RNA-induced silencing complex that binds and unwinds the small interference RNA duplex. The resulting vector, AGO2-GFP1-10, was co-transfected with VSV-G-GFP11 into 293T cells. Another RNA-binding protein, ELAV/HuR, was used as a negative control in this experiment. Resulting Gectosomes were then collected for testing. PTEN induced kinase 1 (PINK1) is a kinase that recruits the E3 ubiquitin ligase Parkin to mitochondria in response to the oxidative phosphorylation uncoupler, CCCP, resulting in acute mitophagy. As expected, in HeLa-Venus-Parkin-RFP-Smac cells without Gectosomes, Venus-tagged Parkin localized diffusely in the cytosol of unstimulated cells and relocated to mitochondria upon CCCP treatment (FIG. 5A). However, cells exposed to Gectosomes carrying PINK1 shRNA showed reduced Parkin accumulation on the mitochondrial surface in response to CCCP. This blockage of Parkin recruitment was observed only with AGO2/shPINK1 Gectosomes and not the ELAV1/shPINK1 Gectosomes; knockdown of PINK1 by AGO2/shPINK1 Gectosomes was confirmed with real-time PCR analysis and immunoblotting (FIGS. 5C and 5D). The imperfect correlation between PINK1 knockdown and Parkin localization could result from the off-target effect associated with each shRNA delivery method (FIGS. 5B and 5C). Nevertheless, these results demonstrate that Gectosomes can be programed with RNA-interfering complexes to inactivate genes of interest selectively.

To investigate whether these Gectosomes can deliver a competent gene-editing complex capable of making targeted changes to target cells' genomes, we collected Gectosomes from 293T cells made by co-transfecting VSV-G-GFP11 and Sa-Cas9-GFP1-10, with or without PINK1 sgRNA. These Gectosomes were incubated with HeLa cells expressing Venus-Parkin. Without PINK1 sgRNA, SaCas9 Gectosomes did not affect Venus-Parkin mitochondrial recruitment. In contrast, cells exposed to SaCas9/PINK1 sgRNA Gectosomes showed a 40% reduction in the number of cells positive for Parkin recruitment (FIGS. 5E and 5F). This was accompanied by a partial reduction of PINK1 expression as determined by western blotting (FIG. 5G). The incomplete effect on PINK1 loss is likely due to the fact that not all gene-editing events cause loss of function in a heterogeneous cell population. In addition to HeLa-Venus-Parkin-RFP-Smac cells, we also incubated SaCas9/PINK1 sgRNA Gectosomes with HeLa cells stably expressing PINK1-EGFP and observed partial loss of GFP signal by flow cytometry and western blot (FIGS. 12C-12E). Sequencing analysis of genomic DNA from edited cells showed variable size deletions near the sgRNA targeting site (data not shown). These results indicate that Gectosomes can be designed to encapsulate RNA interference or gene-editing machinery to alter gene expression.

### Example 9: CD47 Suppresses Gectosome Clearance by Macrophages

Circulating monocytes, macrophages, dendritic cells, and neutrophils remove dead cells, cell debris, exosomes, and ectosomes through phagocytosis (Barclay and Van den Berg, 2014). These phagocytic cells express signal regulatory protein a (SIRPa), which serves as a receptor for CD47, a transmembrane protein present in high levels in tumor cells and normal cells alike. Binding of CD47 to SIRPa triggers a “do not eat me” signal. Previous studies showed that the presence of CD47 on exosomes suppresses their depletion by phagocytosis, resulting in higher exosome levels in the blood. In contrast, CD47 blockade with a nanobody (nb) A4 enhanced macrophage phagocytosis of tumor cells. To test if the CD47-SIRPa system plays a role in Gectosome clearance by macrophages in vitro, we overexpressed Myc and GFP11-tagged mouse CD47 or CD47nb in 293T cells, along with the standard Gectosome components (FIG. 6A).

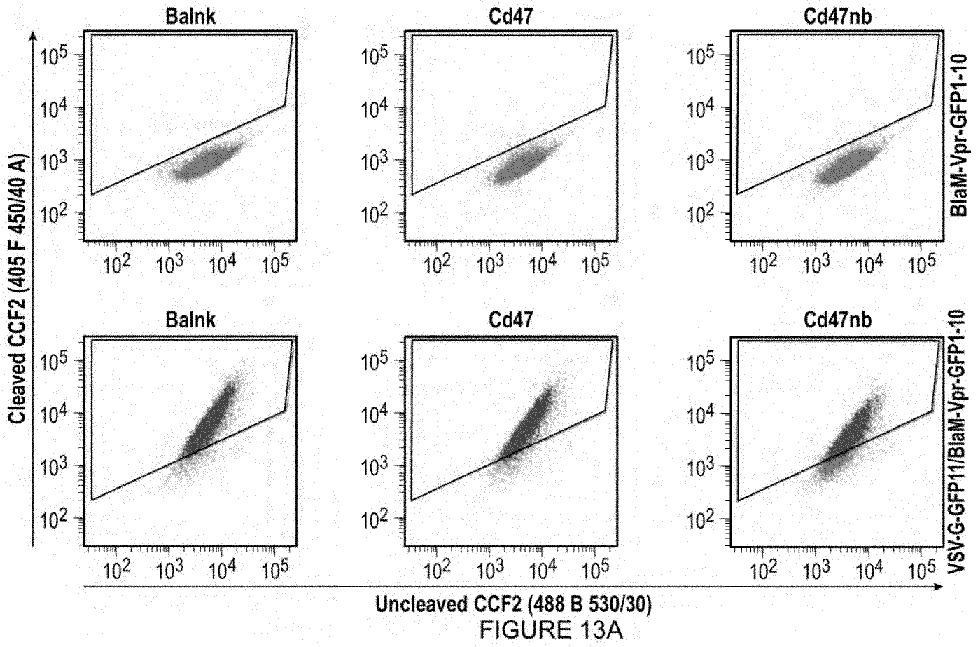

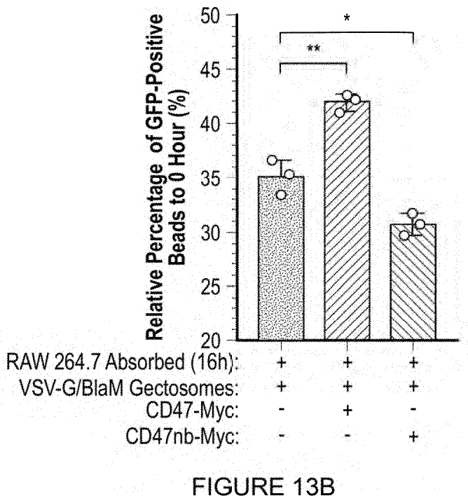

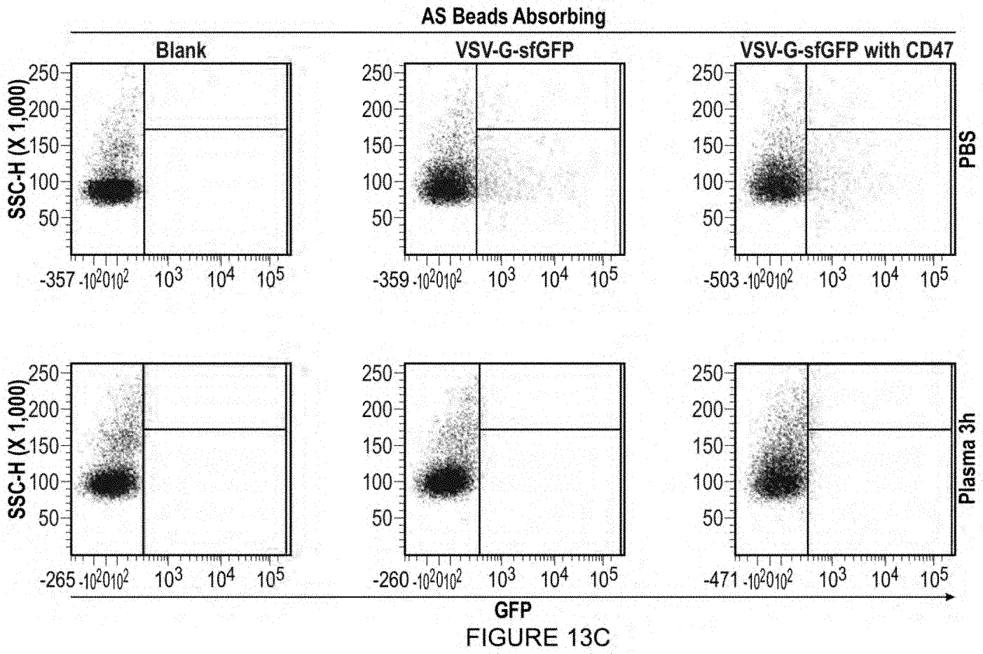

With this design, Gectosomes were generated with higher CD47 or CD47nb expression on their surfaces, along with VSV-G (FIG. 6B). Without VSV-G-GFP11, CD47-Myc-GFP11 or CD47nb-Myc-GFP11 cannot transduce BlaM-Vpr-GFP1-10 to target cells (FIGS. 6C and 13A). Next, we incubated control, CD47, and CD47nb Gectosomes containing BlaM-Vpr-GFP1-10 with mouse RAW 264.7 macrophages for 3 or 6 h. The supernatants were recovered after incubation, and BlaM activity assays measured the amount of Gectosomes remaining in the media. RAW 264.7 cells depleted approximately 25% and 70% of the control Gectosomes after 3 and 6 h (FIG. 6D). In contrast, only 10% and 50% of CD47 Gectosomes were depleted, whereas 70% and 80% of CD47nb Gectosomes were removed from the media. Gectosome depletion was also confirmed by measuring the amount of GFP fluorescent particles left in the supernatants after macrophage exposure (FIG. 13B). The effect of CD47 on Gectosome depletion by macrophage is not reporter specific, as Cre Gectosomes exhibit similar depletion trends (data not shown).

To test if CD47 suppresses Gectosome clearance in circulation in vivo, 4-6-week female BALB/c mice were injected intravenously with VSV-G-sfGFP Gectosomes, produced with or without CD47 (FIG. 6E). The levels of fluorescent Gectosome particles in circulation 3 h post-injection were measured by flow cytometry analysis of aldehyde sulfate beads bound to the Gectosomes in mouse plasma. VSV-G-sfGFP Gectosomes with CD47 showed higher retention in circulation than those without CD47 (FIGS. 6F and 13C). Thus, these results demonstrate that the presence of CD47 on Gectosomes slows their removal by myeloid cells, and conversely, that perturbing CD47-SIRPa interactions accelerates their depletion.

### Example 10: PCSK9 Gene Editing in Mouse Livers Through Systemic Gectosome Delivery of Gene-Editing Machinery

Previous studies have shown that AAV viral delivery of SaCas9 and a sgRNA targeting proprotein convertase subtilisin/kexin type 9 (PCSK9) to mouse liver cells results in a significant reduction of serum PCSK9 and total cholesterol levels. Given concerns about the sustained expression of Sa-Cas9/sgRNA expression in vivo, we wished to determine whether Gectosomes could induce gene editing in somatic tissues of animals through transient delivery of gene-editing machinery. To this end, we first confirmed the editing activity of the two validated sgRNAs targeting PCSK9 used in the previous studies in MEF cells using Gectosome delivery.

To test whether Gectosome could deliver SaCas9/mPCSK9 sgRNA in vivo, we injected Gectosomes (109 per mouse per injection) carrying SaCas9/mPCSK9 sgRNA to BALB/c mice on days 0, 2, and 4. The control group was injected with PBS. Serum PCSK9 and LDL cholesterol levels were measured in blood samples collected on the indicated days (FIGS. 14A and 14C). As early as 14 days after the initial injection, serum PCSK9 levels were significantly lower than those of the control groups (FIG. 14A). This observation was corroborated by immunoblotting for PCSK9 in mouse liver tissues (FIG. 14B). The serum LDL cholesterol levels also correlated with the decline of serum PCSK9 (FIG. 14C). This result suggests that Gectosomal delivery of the SaCas9 gene-editing complex is effective in lowering PCSK9 expression in mouse livers.

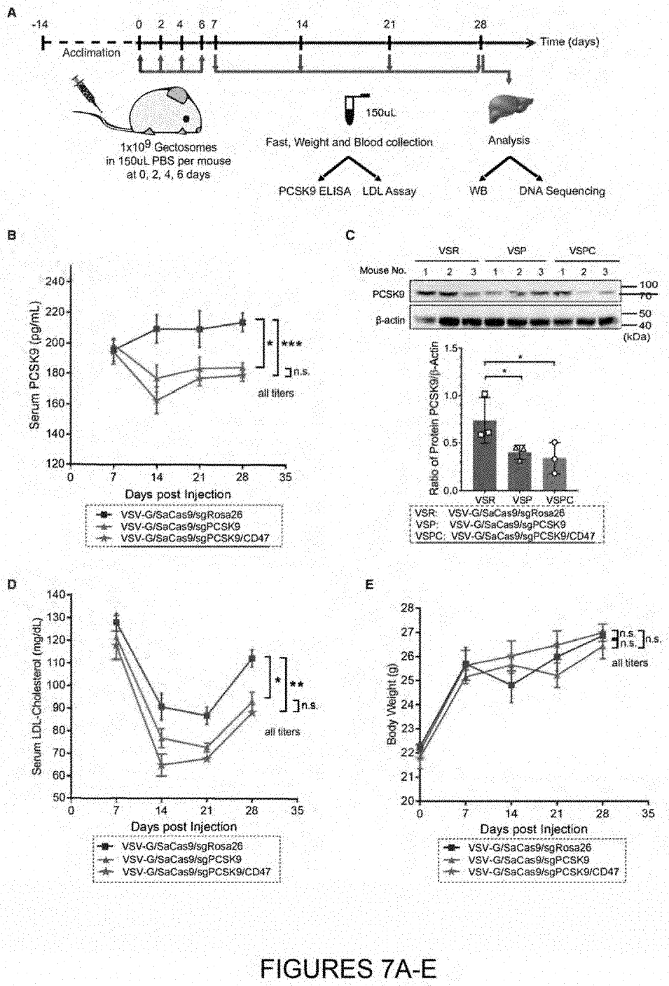

Next, we set out to test whether CD47 can promote Gectosome delivery efficiency in vivo by adding two additional groups: SaCas9 with Rosa26 sgRNA Gectosomes and CD47/SaCas9/mPCSK9 sgRNA Gectosomes (FIG. 7A). The first one serves as the non-targeting control as Rosa26 sgRNA has been used in previous studies. The CD47 group was included based on our in vitro results showing reduced Gectosomes clearance by CD47. Similar to what we observed in the initial study, animals treated with SaCas9/mPCSK9 sgRNA Gectosomes showed a statistically significant reduction in both PCSK9 and LDL from the control with Rosa26 sgRNA (two-way ANOVA test) (FIGS. 7B-7D). The decline of LDL cholesterol levels in all groups between day 14 and day 21 may be caused by procedure-induced stress. The CD47 group showed consistently lower PCSK9 and LDL cholesterol levels and higher statistical significance from the control group, although the difference between this group and that without CD47 was found to be not statistically significant. The dynamics of LDL cholesterol change were unknown, but their separation from the control groups was consistent. There were no significant differences in body weight changes during the experiment between the group of animals (FIG. 7E), suggesting no general systemic toxicity associated with Gectosome injection. To further confirm the PCSK9 mutations caused by SaCas9/mPCSK9 sgRNA Gectosomes, genomic DNA was extracted from liver tissue, followed by PCR and DNA sequencing analyses. The results show that both deletions and mutations can be detected in the Gectosome-treated group (Table S4), confirming that gene editing indeed occurred in vivo upon Gectosome delivery of SaCas9/PCSK9 sgRNA complex. Overall, these results support the potential of Gectosomes to deliver effective genome-editing machinery to animal tissues.

### Example 11: The Versatility of Loading VSV-G Gectosomes with Cellular Proteins

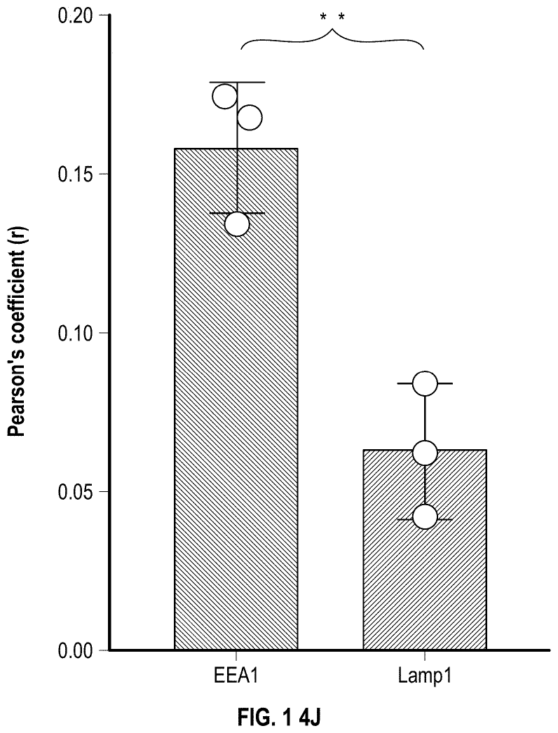

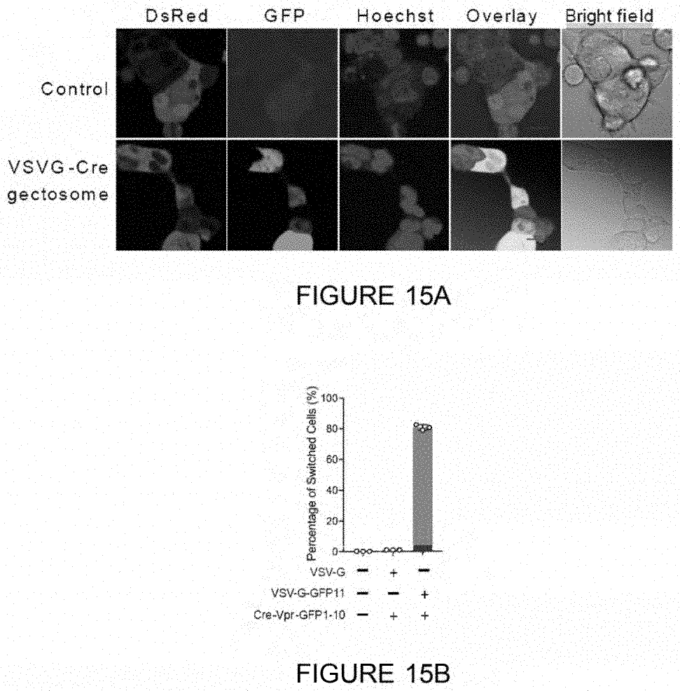

To demonstrate the generality of gectosomes in delivery of a variety of cellular proteins, the present inventors next tested whether Cre recombinase could be efficiently incorporated into gectosomes to deliver bioactive Cre to modify the recipient cells (FIG. 1G). Cre-vpr-GFP1-10 was transiently transfected into HEK293T cells with or without VSV-G-GFP11 (FIG. 9A). NanoSight analysis of media collected from transfected cells revealed that fluorescent Cre gectosomes are relatively more homogenous than BlaM gectosomes based on the NanoSight traces in the FITC channel (FIG. 8B). To test the functionality of Cre delivery by gectosomes, the inventors collected media from HEK293T cells that had been transfected with tethered VSV-G-GFP11/Cre-vpr-GFP1-10, untethered VSV-G/Cre-vpr-GFP1-10, or mock transfection controls. A similar number of gectosomes were incubated with 293ColorSwitch cells, which stably express a color switch reporter gene. Upon Cre gectosome uptake, cells switch from a strong RFP to a GFP signal due to excision of a floxed RFP-STOP cassette that prevents GFP expression (FIG. 1G). More than 80% of 293ColorSwitch cells were switched to GFP with VSV-G-GFP11/Cre-GFP1-10, while mock transfection or untethered VSV-G/Cre-GFP1-10 did not result in detectable changes (FIGS. 2D, and 15A). The color switch as a result of recombination was also independently confirmed by confocal microscopy (FIG. 15B). These results indicate that bioactive Cre recombinase can be efficiently delivered by gectosomes to mediate Cre-lox recombination in target cells and that bridging Cre with VSV-G through split GFP greatly increases the efficiency of Cre delivery to the nucleus of the recipient cells.

Since both BlaM and Cre are proteins of relatively small size, the inventors further explored whether larger proteins can be efficiently incorporated into gectosomes. AGO2, SaCas9, and LwaCas13 were fused to GFP1-10 and co-expressed with VSV-G-GFP11. Flow cytometry and confocal microscopy analyses confirmed that these proteins form a complex with VSV-G-GFP11 mediated by split GFP (FIG. 11A, LwaCas13 not shown). Gectosomes encapsulating these proteins were released from cells, as verified by western analysis with antibodies recognizing VSV-G and encapsulated proteins (FIG. 8F). Without co-expression of VSV-G-GFP11, none of the intracellular proteins were detected in supernatants, suggesting that VSV-G interaction with these proteins is required for encapsulation of these proteins into gectosomes.

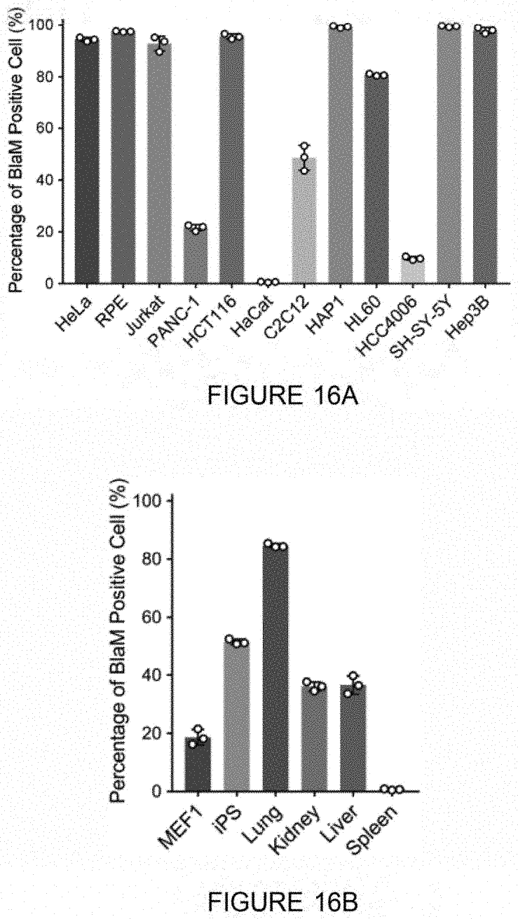

BlaM gectosomes were incubated with several human cancer cell lines and immortalized murine embryo fibroblasts (MEFs). Uptake of gectosomes was found to be very efficient in most of the cell lines tested except HCC4006 and HaCaT keratinocytes (FIG. 16A). Primary cells isolated from mouse showed similar susceptibility to gectosome-mediated cargo transfer (FIG. 16B). Thus, gectosomes can deliver their cargo to many cultured cells and primary cells. Collectively, these results demonstrate that gectosomes can accommodate a variety of cargo proteins and serve as a versatile delivery vehicle.

### Example 12: Dosage and Kinetics of VSV-G Gectosome-Mediated Delivery of Bioactive Proteins in Cultured Cells

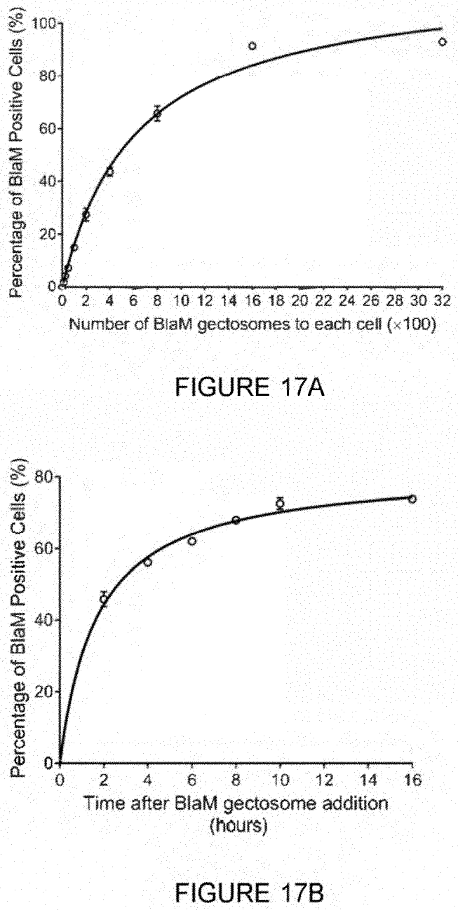

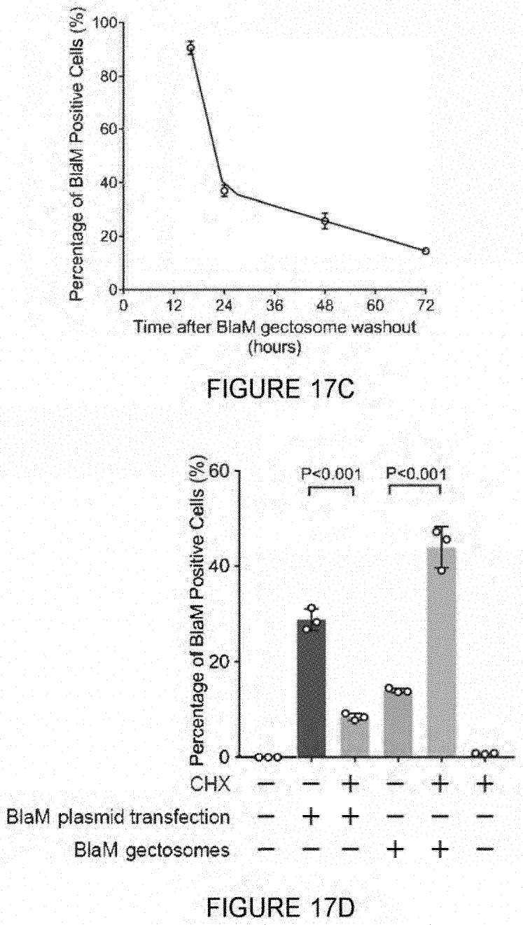

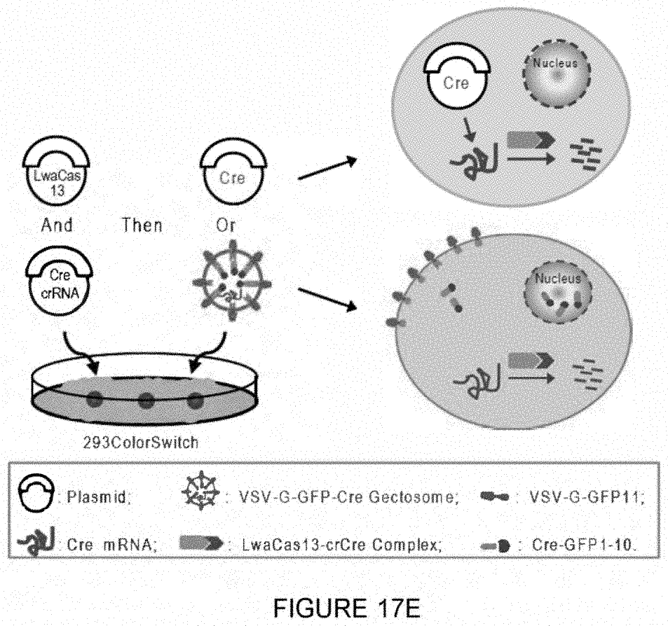

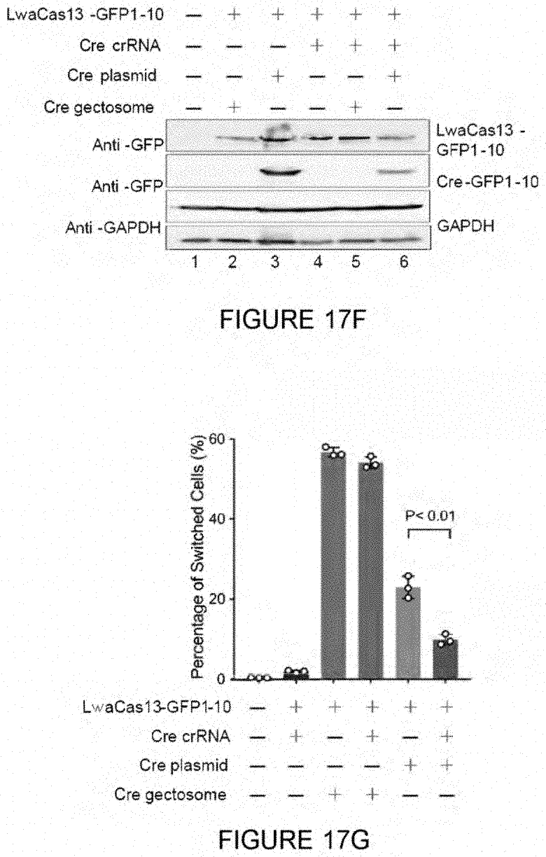

With gectosomes, it may be possible to achieve transient or stable cell modifications in a dose-controlled manner. To assess the dose-dependence of gectosome delivery, an increasing number of fluorescent BlaM gectosome particles were added to a fixed number of HeLa cells for 12 h and measured the fraction of BlaM-positive cells by flow cytometry. Transfer of BlaM to HeLa cells was strictly dose-dependent, with an EC₅₀ of approximately 500 particles per cell (FIG. 17A). Thus, gectosome delivery of bioactive proteins can be dose-controlled.

To investigate the kinetics of gectosomes-mediated protein transfer, the inventors measured BlaM activity in HeLa cells over a period of 16 h after exposure to a submaximal dose of BlaM gectosomes prepared from transfected HEK293T cells. BlaM activity rose rapidly and reached steady-state levels within 8 h post-gectosome exposure in HeLa cells (FIG. 17B). To measure the stability of delivered BlaM, media exchange was performed for HeLa cells loaded with gectosomes at 16 h. In this case, the fraction of cells that retained a BlaM signal was determined for up to 72 h. BlaM signal declined quickly after 24 h and returned to baseline between 48 and 72 h (FIG. 17C). The reduction in BlaM-positive cells is most likely due to the degradation of transferred BlaM enzyme intracellularly. The kinetic profile of this protein when transferred via gectosomes is consistent with the profile after transient delivery of many bioactive molecules.

In addition to proteins, EVs are known to encapsulate nucleic acids, including miRNA, mRNA, and even plasmid DNA. It is possible that the BlaM or Cre function transferred by gectosomes occurs due to the transfer of nucleic acids encoding these proteins as opposed to direct protein transfer, although the rapid rise and decline of BlaM is inconsistent with this hypothesis. To further rule out de novo protein synthesis, the inventors performed a set of experiments using the protein synthesis inhibitor cycloheximide. HeLa cells that were transiently transfected directly with a BlaM expression plasmid or exposed to gectosome-transferred BlaM were treated with cycloheximide (10 μg/mL) or a vehicle control for 16 h prior to flow cytometry analysis. As expected, HeLa cells that had been transiently transfected with an expression plasmid encoding BlaM exhibited less BlaM activity when protein synthesis was inhibited by cycloheximide. In contrast, in HeLa cells into which BlaM protein had been transferred directly using gectosomes, more cells were positive for BlaM expression after cycloheximide treatment, which supports that the BlaM activity after gectosome treatment comes from direct protein transfer rather than new protein synthesis from transferred nucleic acids (FIG. 17D).

To further rule out the possibility of nucleic acid transfer by gectosomes rather than protein transduction, the inventors took advantage of the recently developed LwaCas13-mediated RNA silencing, which confers host cells' innate immunity to invading nucleic acid. The inventors expressed LwaCas13 along with or without 2 tandem sgRNAs targeting Cre in 293ColorSwitch cells (FIG. 17E). In this way, cells were generated that, in the presence of the sgRNAs, are programmed to suppress Cre mRNA. Next the inventors incubated LwaCas13-programmed or unprogrammed cells with Cre gectosomes or directly transfected a Cre expression vector into these cells. As expected, LwaCas13/Cre sgRNA suppressed Cre protein expression in HEK293T cells transfected with the Cre expression plasmid (FIG. 17F, lane 3 versus lane 6), indicating that LwaCas13-mediated Cre knockdown is effective. LwaCas13/Cre sgRNA significantly reduced the RFP to GFP switch in transfected cells, as would be expected with lower Cre expression (FIG. 3G, last two columns). In contrast, gectosome-mediated Cre transfer was not significantly affected by LwaCas13/Cre sgRNA (FIG. 17G, middle two columns). Taken together, these results suggest that gectosome-mediated Cre transduction is unlikely due to DNA or mRNA transfer from the producer cells to the recipient cells.

### Example 13: Alternative Purification of VSV-G Gectosomes

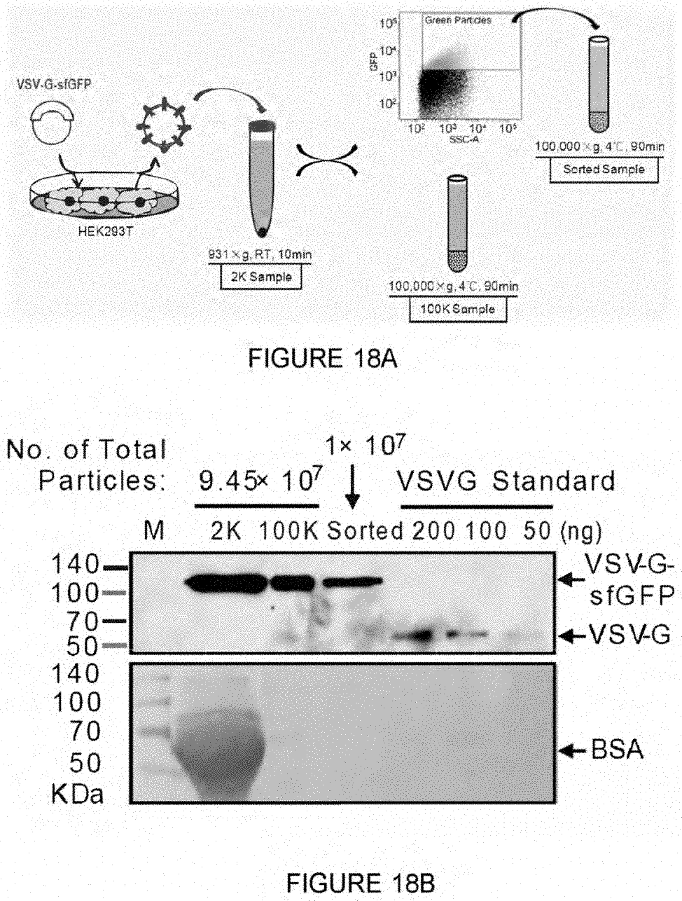

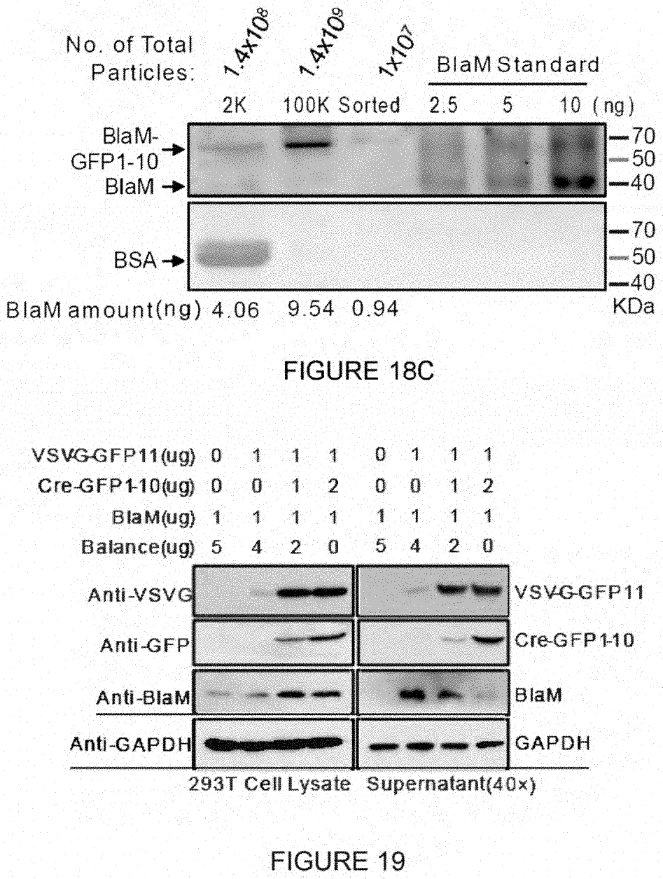

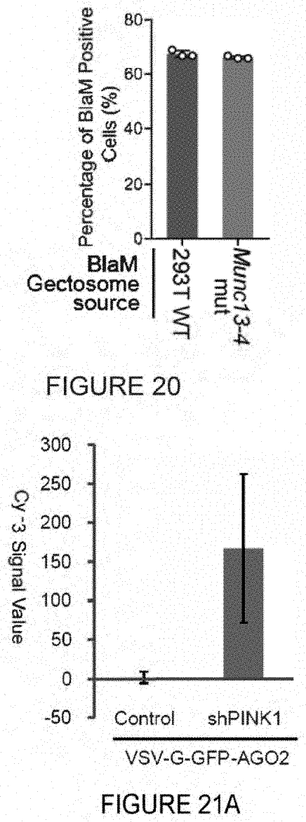

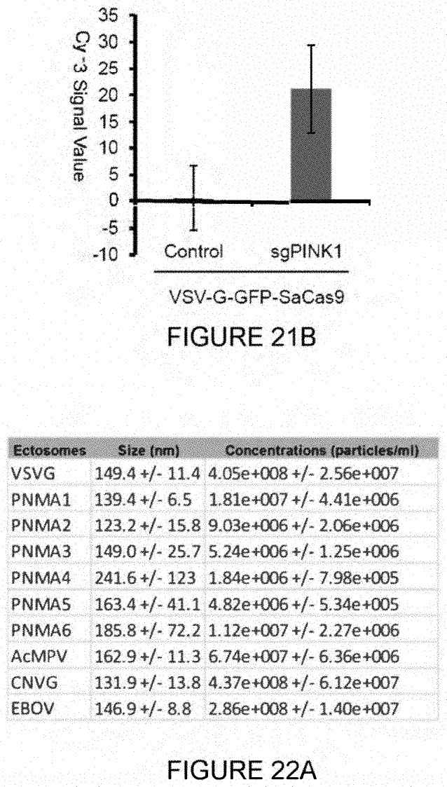

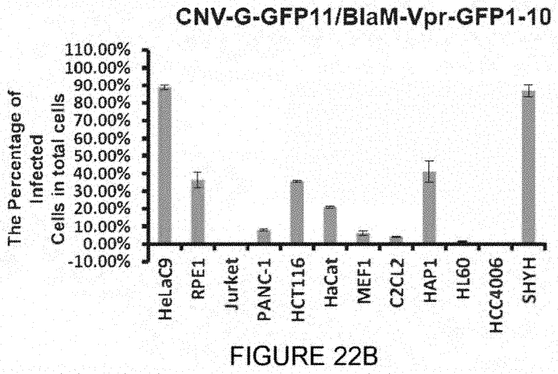

Since flow cytometry can discriminate particles by size and fluorescence, the inventors compared the effectiveness of 100K centrifugation versus FACS for VSV-G-sfGFP gectosome enrichment (FIG. 18A). Quantitative immunoblotting was used to assess the effectiveness of purification and determine the number of protein molecules per gectosome using a known amount of purified recombinant VSV-G or BlaM as the standard (FIG. 18BC). With VSV-G-sfGFP gectosomes, 100K centrifugation and FACS yielded 24 and 72 fold enrichment of VSV-G respectively (Table 1-1). For fluorescent two component gectosomes, the purification table (Table 2-1) shows that 100K centrifugation can achieve ˜33-fold enrichment of BlaM in gectosomes while FACS can achieve ˜467-fold enrichment of the cargo protein BlaM (FIG. 18C and Table 2-1). Thus, the split GFP system enables isolation and purification of desired gectosomes.

### Example 14: Programming VSV-G Gectosomes for Gene Editing in Cultured Cells

In FIG. 17F, the inventors showed that SaCas9-GFP1-10 can be encapsulated into gectosomes and released into media. To investigate whether gectosomes can deliver a competent gene-editing complex to make targeted changes to the genomes of recipient cells, the inventors collected gectosomes made in HEK293T cells by co-transfection of VSV-G-GFP11 and SaCas9-GFP1-10 with or without PINK1 sgRNA. The inventors incubated these with Venus-Parkin HeLa cells. Without PINK1 sgRNA, SaCas9 gectosomes have no effect on Venus-Parkin mitochondrial recruitment. Cells exposed to SaCas9/PINK1 sgRNA gectosomes showed a 40% reduction in the number of cells that are positive for Parkin recruitment (FIGS. 5E and 5F). This is accompanied by a partial reduction of PINK1 expression as determined by western blotting (FIG. 5G). The incomplete effect on PINK1 loss is likely due to the fact that not all gene editing events cause loss of function. The presence of PINK1 sgRNA in SaCas9/sgPINK1 gectosomes was independently confirmed by a custom RNA microarray analysis (FIG. 13B). In addition to Venus-Parkin HeLa cells, the inventors also incubated SaCas9/PINK1 sgRNA gectosomes with HeLa cells stably expressing PINK1-EGFP. Partial loss of GFP signal was also observed after treatment with SaCas9/sgPINK1 gectosomes (FIG. 5).

To confirm whether gene editing indeed occurred at the endogenous PINK1 locus or at the ectopically expressed PINK1-EGFP transgene, the inventors extracted genomic DNA from respective cell lines and performed PCR analysis with a pair of primers amplifying the targeted region. The resulting PCR products were subjected to TA cloning. DNA sequencing of the clones containing the amplified region showed variable size deletions near the sgRNA targeting site (Supplementary Table 1), a pattern consistent with non-homologous end-joining repair of double-stranded breaks to produce these mutations by SaCas9. These results showed that gectosomes packaged with SaCas9 and designed sgRNA can perform gene editing at the endogenous or transgene locus.

### Example 15: Incorporation of p6^{Gag} Peptide Motif to Increase Gectosome Cargo Delivery

The inventors sought to increase cargo delivery efficiency of the gectosomes of the invention. As shown in FIG. 23A, the present inventors generated vectors for production of Gectosomes, including exemplary use of p6^{Gag} peptide motif from human immunodeficiency virus type 1 (HIV-1) Gag protein, to enhance gectosome protein (Cre) cargo delivery efficiency, such constructs comprising: VSV-G-p6Gag-GFP11; VSV-G-GFP11; and Cre-GFP1-10. As shown in FIG. 23B, the present inventors compared the of the efficiency of Cre-GFP1-10 delivery to 293T or HeLa ColorSwitch cells by VSV-G-GFP11 or VSV-G-p6^{Gag}-GFP11. Equal number (1.25×10⁹) of VSV-G-GFP11/Cre-GFP1-10 or VSV-G-p6^{Gag}-GFP11 Gectosome were incubated with ˜1×10⁵ 293T ColorSwitch or HeLa ColorSwitch cells for 48 hr before they were harvested for flow cytometry analysis. Percentage of switched cells is indicated in the plots. Cargo delivery of Cre-GFP1-10 was demonstrated to be at least 20% more efficient with p6^{Gag} variant of VSV-G.

### Example 16: Programming Gectosomes for RNA Interference and RNA Ablation

To demonstrate that gectosomes can package and deliver RNA-interfering and degrading functionality to suppress the expression of a gene of interest in recipient cells, we fused GFP1-10 with Ago2, a component of the RNA-induced silencing complex (RISC) that binds and unwinds the small interference RNA duplex, or with recently discovered LwaCas13a. The Ago2-GFP1-10 or LwaCas13-GFP1-10, were co-transfected into HEK293T cells with VSV-G-GFP11 along with a construct encoding siRNA that targets HBx or a CRISPR crRNA that targets HBx. Hep3B cells, which harbor integrated HBV genomes, were treated with gectosomes carrying anti-HBx siRNA loaded via Ago2 or anti-HBx crRNA loaded via LwaCas13. Knockdown of HBx mRNA by Ago2/siRNA or LwaCas13/crRNA gectosomes and HBx protein was confirmed with qPCR analysis and immnunostaining (FIG. 26). These results demonstrate that gectosomes can be programmed with RNA-interfering complexes to inactivate a gene of interest.

### Example 17: Materials and Methods

Animals: All mouse experiments were performed according to the protocol (No. 2667) approved by the IACUC office of the University of Colorado Boulder and the NIH guidelines. Female BALB/c mice (4 to 6 weeks old) from The Jackson Laboratory were used in Gectosome clearance and genome editing experiments.

Cell Culture: 293T, 293ColorSwitch, RAW 264.7, and HeLa cell lines were obtained from the American Type Culture Collection (ATCC). All cell lines were cultured in Dulbecco's modified Eagle's medium (DMEM) supplemented with 10% FBS, 2 mM glutamine, 100 U/M1 penicillin, and 100 mg/mL streptomycin at 37° C. with 5% CO2 incubation. 293T cells were used as the Gectosome producer cells. HeLa-Venus-Parkin-RFP-Smac and HeLa-PINK1-EGFP have been reported. HeLa cells stably expressing Munc13-4 sgRNA were created using lentiviral particles from 293T cells transfected with lentiviral vector pLentiCRISPRv2-Munc13-4 sgRNA with three co-packaging plasmids. 293ColorSwitch cell line was constructed by stable expression of the Cre reporter. 293T and HeLa cell lines were validated by the University of Arizona Genetics Core Facility.

Gene Expression Constructs: A custom-built plasmid vector, called pBbsr-DEST, which contains the Gateway recombination sites, piggyback recombination sites, and IRES-blasticidin, was used for expressing genes in mammalian cells. pBbsr-DEST was constructed using the backbone of pPBbsr2. For expression of the guide RNAs for SaCas9 or LwaCas13, platform specific scaffolds were subcloned into an entry vector derived from pENTR221 (Invitrogen). Detailed maps of these parental vectors are available upon request. GFP11 and GFP1-10 fragments were first subcloned into pENTR and then recombined into pBbsr-DEST to yield pBbsr-GFP11 and pBbsr-GFP1-10 with stuffers. The cDNAs of VSV-G, VSV-G-NJ, CD9, CD81, CD47, CD47nb, HIVp6Gag, GFP1-10 and GFP11 were obtained by gene synthesis (Twist Biosciences or BioBasic) and subcloned into pBbsr-GFP11. The cDNAs of BlaM-Vpr, Cre, AGO2, Elav and SaCas9 were subcloned into pBbsr-GFP1-10 from their source vectors. sgRNA expression vectors for PCSK9, Rosa26, PINK1, EGFP and Cre were constructed by inserting oligonucleotides synthesized into pEntry-U6-(SaCas9). siRNA PINK1 was synthesized by Dharmacon. sgMunc13-4 expression vector was constructed by inserting annealed oligos corresponding to the region of exon 6 in pLentiCRISPRv2-Puro.

Recombinant Protein Expression: Recombinant His-Flag-tagged Cre protein was expressed and purified in E. coli using pET28a vector and stored in a protein buffer (25 mMHEPES [pH 7.4], 150mMKC1, 10% glycerol, and 1mMDTT). The recombinant Cre activity was determined as 1 U/50 ng according to the protocol from NEB.

Production of Gectosomes: 293T cells were seeded into 100 mm dishes and allowed to reach 70-80% confluence before transfection with polyethylenimine (PEI, 3 mL of PEI per mg DNA). Six hours after transfection, the medium was replaced with 10 mL of fresh DMEM. Supernatants were collected at 24 h or 48 h after transfection. For large scale production of Gectosomes for in vivo studies, Freestyle 293 Expression Medium (Thermo Fisher) was used instead of DMEM. The culture supernatants were harvested 48 h after transfection and then subject to centrifugation at 2,000 3 g for 10 min. The resultant supernatant is referred to as the crude Gectosomes.

Flow Cytometric Analysis of Gectosomes: The size distribution of Gectosomes by flow cytometry using FACSAria Fusion Cell Sorter (BD). The crude Gectosomes were run on a FACSAria Fusion (Rate=20000 events/second) under FITC channel. The size reference beads from a kit (ExoFlow-ONE EV Labeling Kit for Flow Cytometry, SBI System Biosciences) were used as a standard size control. DMEM with 10% FBS, conditional control cultural supernatant, and the crude Gectosomes were diluted with ultrafiltered PBS (100 KDa cutoff Amicon Ultra-15 Centrifugal Filter) to 1:100 and then submitted to flow cytometric analysis. 100,000 particles were collected for each sample. The gate was plotted according to the standard size reference beads where there were two groups colored with FITC. FITC-110 and FITC-500 are referred to as 110 nm and 500 nm size beads. The ratio of GFP-positive Gectosomes was analyzed with BD FACSDiva software.

Particle Size and Concentration Measurement by Nanoparticle Tracking Analysis (NTA): NanoSight NS300 (NanoSight Ltd., UK) equipped with a high sensitivity sCMOS camera and NanoSight NTA 3.0 software was used to measure the size distribution and concentration of total particles of extracellular vesicles following the instructions of the manufacturer. The measurement parameters were as follows: temperature ranging from 21 to 23.6° C.; viscosity between 0.9 and 0.965 cP, measurement time 60 s, and 3 technical repeats (n=3). The measurement threshold was set at a similar level for all test samples. The data of total particles were obtained under the clear scatter measurement. We used 488 nm fluorescent filters to collect the data specific for fluorescent Gectosomes or exosomes. The results were shown as the mean sizes of particles plus standard deviations of three repeats.

Gectosome Release Assay: To confirm the release of Gectosomes from producer cells, we seeded 293T cells into a 6-well plate and transfected at 70-80% confluence with 1 mg of pBbsr-VSV-G-GFP11 plus 2 mg pBbsr-BlaM-Vpr-GFP1-10 or Cre-GFP1-10 or AGO2-GFP1-10 or SaCas9-GFP1-10 using PEI. The media were replaced with 2 mL of fresh DMEM after 6 hr. Cell pellets and culture supernatants were collected 48 hr later. After removal of cell debris through centrifugation at 2,000 3 g for 10 min, the released particles were collected through ultra-centrifugation with a 20% sucrose cushion for 90 min at 100,000 3 g 4° C. The resultant pellets enriched with EVs were resuspended in 50 uL of lysis buffer (50 mM Tris [pH 7.4], 150 mM NaCl, 1% Triton X-100, and a protease inhibitor cocktail (Roche)). The corresponding cell pellets were lysed for 30 min on ice in 100 mL of lysis buffer and clarified by centrifugation for 5 min at 12,000 rpm at 4° C. to separate into the Triton-soluble and -insoluble cellular fractions. The EVs, Triton-soluble, and Triton-insoluble fractions were subjected to SDS-PAGE and immunoblotting analysis.

Immunoblotting Analysis: Protein concentration for cell extracts and vesicles was measured using the BCA assay (Thermo Fisher). Equivalent amounts of proteins were boiled in Laemmli sample buffer, resolved on 12% SDS-PAGE gels, and transferred to a 0.22 mm nitrocellulosemembrane. Membranes were blocked for 1 h in 5% non-fat dry milk (Nestle Carnation) or 5% bovine serum albumin depending on the primary antibody used. The filters were incubated with specific antibodies in Tris-buffered saline, 0.1% Tween 20 (TBST) overnight at 4° C. Antibodies used for Western blotting were as follows: anti-VSV-G (Mouse, 1:1000, Kerafast); anti-GFP (Rabbit, 1:1000, Cell Signaling Technology); anti-BlaM (Mouse, 1:1000, Abcam); anti-PINK1(Rabbit, 1:1000, Cell Signaling Technology); anti GAPDH (1:2000, Santa Cruz Biotechnology); anti-CD9 (Rabbit, 1:1000, Cell Signaling Technology); anti-GM130 (Mouse, 1:1000, Cell Signaling Technology); anti-b-actin (Mouse, 1:2000, Santa Cruz Biotechnology); anti-Actinin4 (Mouse, 1:1000, Santa Cruz Biotechnology); anti-TSG101 (Mouse, 1:1000, Santa Cruz Biotechnology); anti-Annexin V (Mouse, 1:1000, Cell Signaling Technology); anti-Flotillin (Mouse, 1:1000, Cell Signaling Technology). Munc13-4 antibody (Rabbit, 1:1000, R&D systems). For chemiluminescence detection of proteins, HRP-conjugated anti-rabbit IgG (Cell Signaling Technology), and anti-mouse IgG (Cell Signaling Technology) secondary antibodies, and SuperSignal West Dura Substrate (Fisher Scientific) were used. ImageQuant LAS 4000 (GE HealthCare) was used to acquire images of the blots. For quantitative immunoblotting experiments to determine the amount of VSV-G, Cre, and BlaM in Gectosomes, recombinant GFP protein (pro-687) was purchased from ProSpec, and recombinant b-lactamase (RP-431) was purchased from Alpha Diagnostic International. Recombinant Cre was prepared as described above. Serial dilutions of each recombinant protein were quantified by Coomassie blue staining along with a known amount of BSA to derive a standard curve for each protein.

BlaM and Cre Protein Cellular Uptake Assays: The b-lactamase (BlaM) cellular uptake assay was performed following the reported procedure. Briefly, the indicated number of Gectosomes was incubated with HeLa or the mentioned cell lines seeded in 6-well plates for 16 hr or indicated time points. Cells were trypsinized, harvested, and spun at 1000 rpm for 5 min. Cell pellets were resuspended using 50 mL of CCF2-AM labeling solution prepared according to the manufacturer's instructions supplied with GeneBLAzer In Vivo Detection Kit (Thermo-Fisher Scientific). Cells were labeled for 1 h at 25° C. and then washed once with DMEM medium. The labeled cells in 500 mL fresh DMEMmedium were analyzed by flow cytometry using BD FACSCelesta (BD Biosciences). 10,000 cells were collected for each sample. The fluorescence profiles in 488 nm and 405 nm channels were acquired and plotted using BD FACSDiva software. The mean percentages and standard deviations of three repeats were recorded. For measuring Cre cellular uptake, 293ColorSwitch cells seeded on 6-well plates were used as target cells and incubated the indicated number of extracellular vesicles for 48 hr or indicated times. Following the incubation, cells were trypsinized, harvested, and scanned in 595 nm and 488 nm channels by flow cytometry using BD FACSCelesta (BD Biosciences). 10,000 cells were collected for each sample. The results were plotted with BD FACSDiva, and the mean percentages of green cells with standard deviations were recorded with three replicates (n=3).

Purification and Immobilization of the VSV-G Antibody: To purify the anti-VSV-G antibody, hybridoma cell line 8G5F11 (a gift of Douglas Lyles) was cultured in RPMI1640 for 3 days. The resultant supernatant was harvested by centrifugation at 2,0003 g for 5 min and subsequently filtered using a 0.2 mm filter to remove smaller cell debris. The cleared supernatant was incubated with Protein G-Agarose beads or Protein G-magnetic beads (Thermo Fisher) overnight. The antibody-bound beads were washed with PBS for 5 min three times, eluted with 100 mL 0.1 M Glycine [pH 2.7], and neutralized with 1 M Tris [pH9.5]. For conjugation of the VSV-G antibody to Protein G-Agarose beads or Protein G-magnetic beads, beads were incubated with the purified antibody overnight at 4° C. and then washed with PBS to remove the unbound antibodies. Freshly made DMP solution (13 mg/mL Dimethylpimelimidate in Wash buffer, pH8-9) was added to beads at 1:1 ratio, and the mixture was rotated for 30 min at the room temperature. The conjugated beads were washed three times with the Wash buffer (0.2 M triethanolamine in PBS) at room temperature for 5 min per cycle. The conjugated beads were resuspended in PBS, and the labeling reaction was stopped by adding an equal volume of the quench buffer (50 mM ethanolamine in PBS). The beads were washed with 0.1 M glycine [pH 2.7] for 10 min twice and stored in PBS with 20% ethanol at 4° C. until further use.

Nanosight analysis of VSV-G gectosomes: VSV-G fused with GFP11 and cargo genes fused with GFP1-10 were expressed and combined in HEK293T cells so that secreted VSV-G enveloped gectosomes with cargo proteins showed GFP signal under NanoSight analysis. Raw released VSV-G gectosomes from HEK293T culture supernatant were assayed to measure the size distribution and concentration of total particles and VSV-G gectosome using NanoSight NS300 (NanoSight Ltd., UK) equipped with a sCMOS camera and NanoSight NTA 3.0 software. The measurement conditions were as follows: temperature between 21 and 23.6° C.; viscosity between 0.9 and 0.965 cP, measurement time 60 s and 3 repeats. The measurement threshold was similar in all samples. The data of total particles were obtained under the clear scatter measurement. The inventors used 488 nm fluorescent filters to block out the scattered laser light and only image the fluorescent signal coming from the VSV-G gectosomes to measure the size distribution. The results indicate the mean sizes of particles and standard deviations of three repeats.

### Isolation and Purification of Gectosomes

Isolation of EVs by Ultracentrifugation (UC): The conditioned medium collected from cells growing on 100 mm plates was first cleared by low-speed centrifugation at 2,0003 g for 10 min (2 K sample) to remove cell debris. The supernatant was centrifuged at 10,0003 g at 4° C. for 30 min (10 k pellet), transferred to new tubes, and ultracentrifuged at 100,0003 g in an SW41Ti (Beckman Coulter) at 4 C for 90 min (100 k pellet).

Isolation of Gectosomes by Immunocapture: Cell conditioned medium was collected from confluent control or transfected 293T cells grown in Freestyle 293 Expression Medium on 100 mm culture dishes at 24 hr or 48 hr after transfection. The medium cleared by centrifugation at 10,0003 g for 30 min at 4° C. was incubated with magnetic beads or agarose beads containing the crosslinked 8G5F11 VSV-G antibody (8G5F11) at 4° C. overnight. Beads bound with Gectosomes were washed with PBS for 5 min three times. Gectosomes were eluted with 0.1 M Glycine [pH 2.7] and then neutralized with 1 M Tris [pH9.5].

Purification of Gectosomes: 0.5 mL of the cell-conditioned medium was processed as the above, except that supernatant from the 10,000 3 g spin was loaded onto IZON qEVoriginal column (IZON Science). Fractions (500 mL each) were collected using an Automatic Fraction Collector (IZON Science). Gectosomes were eluted in fractions 2 and 3, as determined by flow cytometry and immunoblotting analyses. Fraction 2 and 3 were combined and incubated with magnetic beads containing the crosslinked 8G5F11 VSV-G antibody. Gectosomes were eluted with 0.1 M Glycine [pH3.7] and then neutralized with 1 M Tris [pH9.5].

Fluorescence Microscopy: 293T cells were seeded in a 96-well plate with glass bottom at 50% confluence and then transfected with plasmids encoding the split GFP system. 24 hours after transfection, cells were fixed with 2% paraformaldehyde in PBS containing DAPI (1.5 mg/ml DAPI). For imaging Gectosomes uptake, HeLa cells were incubated with Gectosomes for indicated times before fixation with 2% paraformaldehyde in PBS containing DAPI (1.5 mg/ml DAPI). Cells were stained with indicated primary and secondary antibodies before they were imaged with a laser scanning confocal microscope (Nikon MR). For the fluorescence switch imaging, 293ColorSwitch cells were seeded in a 96-well plate with glass bottom at 50% confluence before incubation with VSV-G/Cre Gectosomes. After 48 hours, cells were stained with Hoechst 33342 and imaged described above.

Negative Stain Transmission Electron Microscopy and Immunogold Labeling: TEM imaging and sample preparation were performed at the Electron Microscopy Services Core Facility of the University of Colorado Boulder.

Negative Stain TEM: Purified sfGFP and Cre Gectosomes through immunoaffinity procedure were applied to the negative stain. Briefly, 5 mL of the samples were firstly fixed in 4% paraformaldehyde for 1 hour, applied on a discharged, carbon-coated 400-mesh copper grid, and left it on for 3-5 minutes. The grid was washed in 1 mM EDTA, and then 10 mL 0.75% uranyl formate is to be used for 1 minute for staining. The grid was subjected to TEM imaging.

Immunogold Labeling TEM: Briefly, for immunogold labeling with anti-VSV-G, purified Gectosomes through immunoaffinity procedure were fixed for 1 h in 4% paraformaldehyde and then applied to a discharged, carbon-coated 400-mesh grid. The grids were then put onto a droplet of 1 M Ammonium Chloride for 30 minutes. The samples on the grid were applied to immunogold labeling. The grids were rinsed for 5 min on large droplets of TBS-Tween (50 mM TBS, 0.05% Tween 20, [pH 7.6]) for three times. The grids were incubated in block solution (1% BSA, 3% normal serum, 0.1% Fish Gelatin, 0.05% Sodium Azide in 0.05 M TBS, [pH 7.6]) for 30 minutes. Then the grids were put into droplets of VSV-G antibody (1:50) (or mouse serum as control) diluted in block solution for 1 hour at room temperature. After rinsed the grids in large droplets of TB S-Tween for 5 min for three times, the grids were incubated in droplets of goat anti-mouse IgG/M 6 nm (1:40) for 1 hour at room temperature. The samples on the grids were rinsed in droplets of TBS-Tween for 5 min three times. Lastly, a negative stain was performed as mentioned above. The images were recorded on a 120 kV Tecnai G2 Spirit transmission electron microscope at 52,000 3 magnification.

Recombinant Cre Liposome Preparation: The preparation of recombinant Cre liposome was performed following the published procedure. Lipids used in this work were purchased from Avanti Polar Lipids. Briefly, 1-palmitoyl-2-oleoyl-sn-glycerol-3-phosphocholine (POPC), 1-palmitoyl-2-oleoyl-sn-glycerol-3-phosphoethanol amine (POPE), 1-palmitoyl-2-oleoyl-sn-glycerol-3 phosphoserine (POPS), and cholesterol were mixed in a molar ratio of 60:20:10:10. Cre proteoliposomes were prepared by the detergent dilution method. Complete detergent removal was achieved by overnight dialysis using Novagen dialysis tubes against the reconstitution buffer (25 mM HEPES [pH 7.4], 100 mM KCl, 10% glycerol, and 1 mM DTT) followed by liposome flotation on a Nycodenz gradient. The final concentration of Cre encapsulated in the liposome was determined by immunoblotting analysis using a serial dilution of a known amount of Cre as the standard.

Mass Spectrometry Analysis: Gectosomes immunocaptured on beads were boiled with 30 mL 1% SDS in 100 mMTris-HCl [pH7.3] for 10 min and then submitted to mass spectrometry analysis. The samples were reduced and alkylated in 50 mM Tris-HCl, pH 8.5, containing 4% (w/v) SDS, 10 mM Tris(2-carboxyethylphosphine) (TCEP) and 40 mM chloroacetamide by boiling at 95° C. for 10 min. Samples were then digested using the SP3 method. Briefly, carboxylate-functionalized speedbeads (GE Life Sciences) were added to the extracts and then acetonitrile was added to 80% (v/v) to precipitate proteins onto the beads. The beads were washed twice with 80% (v/v) ethanol and twice with 100% acetonitrile. Proteins were digested on the beads in 20 mL 50 mM Tris-HCl [pH 8.5] and 0.5 mg Lys-C/Trypsin (Promega) incubating at 37° C. for 18 hours with shaking at 1000 rpm. Digestion buffer was removed by adding acetonitrile to 95% (v/v) again, precipitating tryptic peptides onto the beads, followed by washing the beads once with acetonitrile. Peptides were removed from the beads in 50 mL 1% (v/v) trifluoroacetic acid and 3% (v/v) acetonitrile, then dried in a vacuum concentrator and stored at −20° C. Samples were suspended in 15 mL 0.1% (v/v) trifluoroacetic acid and 3% (v/v) acetonitrile then 5 mL was directly injected onto a C18 1.7 mm, 130A°, 75 mm×250 mm M-class column (Waters) using a Thermo Scientific Ultimate 3000 RSLCnano UPLC. Peptides were eluted at 300 nL/minute using a gradient from 3% to 7% acetonitrile in 4 min, then 7% to 24% acetonitrile over 36 min into a Q-Exactive HF-X mass spectrometer (Thermo Scientific). Precursor mass spectra (MS1) were acquired at a resolution of 60,000 from 380 to 1580 m/z with an AGC target of 3×106 and a maximum injection time of 45 ms. Precursor peptide ion isolation width for MS2 fragment scans was 1.4 m/z sequencing the top 12 most intense ions. All MS2 sequencing was performed using higher-energy collision dissociation (HCD) at 27% collision energy and scanning at a resolution of 15,000. An AGC target of 105 and 40 s maximum injection time was used for MS2 scans. Dynamic exclusion was set for 30 seconds with a mass tolerance of +/−10 ppm. MS data files were searched against the Uniprot human database downloaded Nov. 13, 2019 with three additional sequences for VSV-G/GFP, b-lactamase and Cre using Maxquant version 1.6.3.4. Cysteine carbamidomethylation was set as a fixed modification, while methionine oxidation and protein N-terminal acetylation were set as variable modifications. All peptides and proteins were filtered at a 1% false discovery rate (FDR). Proteins identified by one or more specific peptides in either VB or VCB samples (Q<0.001) were included in Table S2 for the analysis.

RNA Interference by Gectosomes: Gectosomes loaded with PINK1 shRNA were produced by transient transfection of 293T cells with PINK1 shRNA plasmid along with expression plasmids VSV-G-GFP11 and AGO2-GFP1-10 or Elav-GFP1-10. The conditional culture supernatant containing Gectosomes (˜3×108 particles/mL) was harvested, and 2 mL was incubated with target HeLa-Venus-Parkin-RFP-Smac cells (˜3×105 cells/well in a 6-well plate). After 24 hours, the culture supernatant was replaced with 2 mL of fresh media. After 3 days, Parkin localization on mitochondria in HeLa-Venus-Parkin-RFP-Smac cells in response to CCCP treatment was analyzed by method described below. Total RNAs were isolated using the Trizol method (Thermo Fisher Scientific). The levels of PINK1 mRNA was measured by RT-qPCR analysis. The primers used in RT-qPCR were listed below:

(PINK1, Forward; SEQ ID NO. 3)
5′-CACCGCCTGGAGGTGA CAAAGAGCA-3′,
(PINK1, Reverse; SEQ ID NO. 4)
5′-AAACTGCTCTTTGTCACCTCCAGGC-3′.

GAPDH gene was used as the control. The GAPDH primers used were listed below:

(Forward SEQ ID NO. 6)
5′-GAAGGTGAAGGTCGGAGT-3′
and
(Reverse; SEQ ID NO. 6)
5′-GAAGATGGTGATGG GATTTC-3′.

Immunoblotting was used to probe the PINK1 protein level.

Genome Editing with CRISPR/Cas9 Gectosomes: Gectosomes encapsulated with SaCas9-sgPINK1 (or mouse sgPCSK9) were produced by transient transfection of 293T cells with VSV-G-GFP11, SaCas9-GFP1-10 with guide RNA encoding plasmid sgPINK1 or mouse sgPCSK9. For PINK1 editing in human cells, the conditioned culture supernatant containing the indicated Gectosomes (108 particles/mL) was harvested, and 2 mL of the supernatant was incubated with HeLa-Venus-Parkin-RFP-Smac and HeLa-PINK1-EGFP cells (3×105 cells/well in a 6-well plate). After 24 hours, the culture supernatant was replaced with 2 mL of fresh media. After treatment for five days, Parkin localization on mitochondria, protein levels, and mRNA levels of PINK1-EGFP in HeLa-Venus-Parkin-RFP-Smac and HeLa-PINK1-EGFP cells as described above. For PCKS9 gene editing in mouse cells, MEF cells were incubated with Gectosomes loaded with SaCas9 and sgPCSK9. To determine whether PINK1 or PCSK9 was edited in cells exposed to Gectosomes, the genomic DNA of the treated cells was extracted using the Blood and Tissue DNA Extraction kit (Qiagen) following the manufacturer's instructions. The primer sequences for PINK1 gene target are:

(PINK1Ex1, Forward, for Exon; SEQ ID NO. 7)
5′-CGCTGCTGCTGCGCTTCA-3′
(PINK1Ex3, Reverse, for Exon; SEQ ID NO. 8)
5′-CTGCTCCATACTCCC CAGCC-3′,
(PINK1Int2, Forward, for intron; SEQ ID NO. 9)
5′-GTCTCCATAATCAGACACCT-3′
(PINK1Int3, Reverse, for intro; SEQ ID NO. 10)
5′-GGATGGTGAACTAACCAATC-3′,
(mPCSK9, Forward; SEQ ID NO. 11)
5′-GATGCCACTTTACTTCGGAGGA-3′
(mPCSK9, Reverse; SEQ ID NO. 12)
5′ AGGAGGATTGGAGTGGGGATTA-3′.

PCR products were recovered and cloned using a TOPO TA Cloning Kit (Invitrogen). The colonies with insert fragments were sequenced and aligned with wild type genomic sequences, respectively.

Gectosome Clearance by Macrophage Cells: Gectosomes with CD47 or CD47nanobody were prepared from 293T cells seeded on 100 mm plates by transfecting 5 mg VSV-GGFP11 plus 10 mg BlaM-Vpr-GFP1-10 with 5 mg CD47-GFP11 or 5 mg CD47nanobody-GFP11 plasmids. Gectosomes were harvested 48 hr post-transfection and cleaned up at 2,000 3 g for 10 min. Next, Gectosomes were incubated with RAW 264.7 cells for the indicated period. RAW 264.7 cells were subsequently removed to recover the supernatants, which were subsequently incubated with HeLa cells for 16 hr before the BlaM activity was measured by flow cytometry, as described above. To directly measure the depletion of Gectosomes by macrophage, the particles were coupled to Aldehyde/Sulfate beads using a protocol for flow cytometric analysis. Briefly, the supernatants recovered after incubation with RAW 264.7 cells were ultracentrifuged for 1.5 hr at 100,0003 g at 4° C. twice. The pellets were then resuspended in 200 mL PBS plus 10 mL of Aldehyde sulfate beads (Aldehyde/Sulfate latex, 4% w/v 4 mm, Invitrogen). 600 mL of PBS was then added to the mixtures and kept at 4° C. on a tumbler overnight. Then 1 M glycine (400 mL) was added to the mixture and incubated at room temperature for 1 hr. Beads were collected by brief centrifugation and washed three times with PBS plus 10% FBS before resuspended in 1 mL PBS with 10% FBS. The fluorescence intensity of Gectosomes immobilized on the beads was measured by flow cytometry.

Gectosome Clearance in Mice: To measure the Gectosome level in circulation in vivo, female BALB/c mice (4 to 6 weeks old) were injected intravenously with sfGFP Gectosomes produced with or without CD47 in 293T cells. The concentration of sfGFP positive Gectosomes in the supernatant was determined by NTA. Gectosomes were buffer-exchanged and concentrated to 1010 particles in 150 mL of PBS using ultrafiltration with the 100 KDa cutoff Amicon Ultra-15 Centrifugal Filters). Concentrated Gectosomes were injected into BALB/c mouse (3 mice each group) through the tail vein. Three hours after injection, the injected mice were sacrificed to collect the EDTA-anticoagulated blood (150 mL) from mouse orbit. The blood samples were kept at room temperature for 1 hr prior to collecting the plasma by centrifugation at 3,000 rpm for 10 min at 4° C. Plasma (150 mL) was diluted to 5 ml with PBS and ultracentrifuged 1.5 hr at 100,0003 g at 4° C. twice. The pellet was resuspended in PBS and mixed with aldehyde sulfate beads as described above. The rate of Gectosome depletion was measured by flow cytometry.

Genome Editing in Mice: Female BALB/c mice (4 to 6 weeks old) were ordered from The Jackson Laboratory. For the investigation of whether Gectosome delivery of the SaCas9-sgPCSK9 gene editing complex, the control and PCKS9 Gectosomes were prepared by transient transfection of 293T cells growing in Freestyle 293 Expression Medium. Gectosomes were concentrated approximately 100 fold by ultrafiltration using 100 KDa cutoff Amicon Ultra-15 Centrifugal Filter Unit. Gectosomes were injected into 4-week-old female BALB/c mice via the tail vein. All dosages of Gectosomes were adjusted to 150 mL containing approximately 109 fluorescent Gectosomes in sterile phosphate-buffered saline. Mice received 109 particles/150 mL each tail vein injection for four times at 48 h of interval. For the measurement of the serum levels of PCSK9 and LDL-cholesterol, animals fasted overnight for 15 hr before blood collection by saphenous vein bleeds. Approximately 50 mL blood was collected from each mouse every 10 days after injection. The serum was collected and stored at −20C for subsequent analysis. Thirty days after injections all mice were sacrificed by carbon dioxide inhalation followed by cervical dislocation, and liver tissue samples were collected and stored at −80C for subsequent DNA or protein extraction. The level of PCSK9 protein in serum was determined by ELISA using a commercial ELISA kit (Mouse Proprotein Convertase 9/PCSK9 Quantikine ELISA Kit, MPC-900, R&D Systems) following the manufacturer's instructions. Serum LDL-cholesterol level was measured using a Mouse LDL-Cholesterol kit (Crystal Chem) per the manufacturer's instructions. Genomic DNA from mouse livers was isolated, and the PCSK9 gene-editing analysis was carried out as described above.

### Mathematical Modeling of Gectosomes

Estimation of Protein Numbers in Gectosome: To determinate the abundance of VSV-G-GFP11 and Cre-GFP1-10 in Gectosome, we performed quantitative Western blot experiments and measured the expressed amount of VSV-G-GFP11, Cre-GFP1-10 proteins with recombinant protein stands. As shown in FIG. 11E, there is about 7.47310⁻⁷ ng VSV-G-GFP11, 9.73310⁻⁸ ng Cre-GFP1-10 present per Gectosome when 293T cells were transfected with 1 mg VSV-G-GFP11, 2 mg Cre-GFP1-10, and 1 mg BlaM plasmids. Therefore, in a Gectosome, we estimate that:

$$\begin{matrix}\# VSV - G - GFP 11 = \frac{7.47 \times {10}^{- 7} \times 1 - {10}^{- 8}}{8.0 \times {10}^{- 8}} \times 6.02 \times {10}^{\text{?}} = 562 \times {10}^{\text{?}} molecules . & ( Equation 1 ) \\ \# Cre - GFP 1 - 10 = \frac{9.73 \times {10}^{- 8} \times {10}^{- 8}}{6.28 \times {10}^{?}} \times 6.02 \times {10}^{\text{?}} = 9.33 \times {10}^{\text{?}} molecules . \text{?} \\ \text{?} \text{indicates text missing or illegible when filed} & ( Equation 2 )\end{matrix}$$

3D Modeling for the Space-Filling of VSV-G-GFP11 and Cre-GFP1-10 Proteins in Gectosomes: The average measured diameter of single Cre Gectosome vesicles in this study is about 185 nm (FIG. 9B). Here we assume that Gectosome vesicles are spherical. Considering that the bilayer thickness is about 2.5-3.5 nm and one side of the lipid head is about 1 nm (Andersen and Koeppe, 2007), we estimate that the membrane thickness of a Gectosome is about 5 nm. If we model VSV-G protein into the membrane in the Orientations of Proteins in Membranes (OPM) database, the VSV-G protein attaches to the outer membrane surface of the vesicle. Cre-GFP1-10 will form a complex with VSV-G-GFP11 through an assumed irreversible complementation process; they are attached to the inner membrane of the Gectosome. We retrieved the protein structures of VSV-G monomer protein structure (PDB ID: 5I2S), sfGFP protein structure (PDB ID: 2B3P) and Cre recombinase monomer (PDB ID: 1NZB) in Blender with ePMV plugin, which show the following dimensions of the bounding boxes.

(1) VSV-G, x: 5.7 nm, y: 4.9 nm, z: 10.2 nm

(2) sfGFP, x: 5.3 nm, y: 4.8 nm, z: 5.0 nm.

(3) Cre recombinase monomer, x: 7.4 nm, y: 6.5 nm, z: 5.4 nm.

FIG. 11E illustrates the relative size and orientation of different protein structures in a Gectosome.

The Occupancy of VSV-G Proteins at the Surface of Gectosomes: Based on the structure of VSV-G (PDB ID: 512S) monomer, the center of VS V-G was measured with a dimension of 5 nm (x-axis) and 4 nm (y-axis) and it is about 100 nm away from the center of Gectosome. We approximate this area as a circle with a diameter of 4.5 nm. Therefore, 5.62310³ VSV-G proteins will occupy 71.1% of Gectosome surface based on the following calculation:

$$\frac{5.62 \times {10}^{3} \times \text{?}}{4 \times {( 100 )}^{2}} = 71.1 \% \\ \text{?} \text{indicates text missing or illegible when filed} \qquad ( Equation 3 )$$

The Occupancy of Cre-GFP Proteins Close to the Inner Membrane of a Gectosome: Based on the parameters shown in FIG. 11E, we can estimate the volume of the intra-Gectosome sphere (Vt; excluding membrane and the volume for the bounding hollow sphere that completely contains Cre and complemented GFP proteins (V^{Cre-GFP}).

$$\begin{matrix}{V}_{\text{?}}^{Cre - GFP} = \frac{4}{3} \text{?} {( 77.1 + 5 + 5.4 )}^{3} - \frac{4}{3} \text{?} {( 77.1 )}^{3} = 8.86 \times {10}^{\text{?}} {( nm )}^{3} & ( Equation 4 ) \\ V \text{?} = \frac{4}{3} \text{?} {( \frac{185}{2} - 5 )}^{3} = 2.81 \times {10}^{\text{?}} {( nm )}^{3} \\ \text{?} \text{indicates text missing or illegible when filed} & ( Equation 5 )\end{matrix}$$

To estimate the space that is occupied by Cre and complemented GFP complex, we use protein bounding volume (Fonseca and Winter, 2012). The bounding volume for one Cre monomer and one sfGFP protein is:

V_{b}^{Cm-GFP}=7.4×6.5×5.4×5.3×4.8×5=387 (nm)³ (Equation 6)

The total bounding volume that 933 Cre-GFP molecules is

$${V}_{\text{?}}^{Cre - GFP} = 9.33 \times {10}^{3} \times 367 = 3.6 \times {10}^{3} {( nm )}^{3} \\ \text{?} \text{indicates text missing or illegible when filed} \qquad ( Equation 7 )$$

which is about 40.7% of the volume of bounding hollow sphere for Cre-GFP proteins (V^{Cre-GFP}) and 12.9% of all the intra-Gectosome space volume (V^{t}).

3D Space-Filling of VSV-G-GFP11 and Cre-GFP1-10 Molecules in a Gectosome: We used an open-source 3D software Blender (blender.org) and the ePMV add-on to model the complemented VSV-G-GFP11 and Cre-GFP1-10 molecules in a Gectosome. The model is based on the space-filling model of the corresponding PDB protein structures at the nanometer scale. The protein structure of VSV-G, GFP11, and Cre-GFP1-10 monomers were represented with “Coarse Molecular Surface” by importing corresponding PDB structure file to ePMV in Blender. The unknown linker domains (e.g.: transmembrane domain) of the fused proteins were simplified as a cylinder, which links the VSV-G in the extra-Gectosome and the complemented sfGFP/Cre proteins. The outside view and the middle intersection view of the 3D model are illustrated in FIG. 3F.

Estimation of the Partial Specific Volume for Proteins Identified from Mass Spectrometry Data: To quantify the relative abundance of the proteins found in the Gectosome, we used the label-free quantitation (LFQ) method based on the mass spectrometry data. The relative molar abundance of a protein was calculated by normalizing their LFQ. values to the LFQ value for VSV-G protein. Based on the measured absolute abundance of VSV-G protein in Gectosome (5.62310³ molecules/Gectosome), we can convert the relative molar abundance to absolute quantification.

$$\# {P}_{i} = \frac{LFQ ( {P}_{i} )}{LFQ ( VSVG )} \times 5.62 \times {10}^{3} \qquad ( Equation 8 )$$

Here we used the following empirical equation to calculate the partial specific volume of proteins based on their molecular weight (Erickson, 2009).

V(nm³)=1.212×10−³ (nm³/Da)×MW(Da) (Equation 9)

To calculate the intra-Gectosome and extra-Gectosome protein volumes, we first identified the vesicle membrane proteins in the MS data using online software DAVID Bioinformatics Resources 6.8 (david.ncifcrf.gov/). The predicted partial specific volumes of the proteins in a Gectosome are listed in Table S2.

Image Analysis: The size of Gectosomes from TEM image was measured by using SerialEM software on transmission electron microscopy. The quan-titative analysis of the colocalization of complemented split GFP (VSV-G-GFP11/Cre-GFP1-10) and EEA1(n=37) or Lamp1(n=47) in HeLa cells were performed using the Jacop Plugin of ImageJ and the data are expressed as a Pearson's coefficient (r).

Statistical Analysis: Statistical analyses of data were performed with GraphPad Prism 7. Data are represented as mean±standard deviation or mean±SEM or average±standard error as indicated. For comparisons between two groups, statistical significance was determined using an unpaired student's t-test. The two-way ANOVA was used to compare the effects of different groups of treatments on PCSK9, LDL-cholesterol, and body weight of animals. Parkin localization on mitochondria was assessed with the MetaXpress application module called Transfluor Cell Scoring Application Module (Molecular Devices). Flow cytometric analysis was typically performed in three technical replicates (n=3) and the number of biological replicates is indicated for specific experiment in figure legends. Specific sample sizes, including the number of particles, cells, mice in each experiment, and p-values are indicated in figures and figure legends.

SUPPLEMENTAL TABLE 1
The evaluation of the protein number each Cre Gectosome from FIG. 3C.

| | | | Amount of | Amount in each | Protein No. in |
|---|---|---|---|---|---|
| | TotalGectosome | Amount of UC | Fraction 2 | Gectosome | each Gectosome |
| Proteins | No. | (ng) | (ng) | (ng/Gecto.) | (/Gecto.) |
| VSV-G-GFP 11 | 1.00E+09 | 742.0 | 747.0 | 7.47E−07 | 5.62E+03 |
| Cre-GFP1-10 | 1.00E+09 | 601.0 | 97.3 | 9.73E−08 | 9.33E+02 |

SUPPLEMENTAL TABLE 3
The volume information of Gectosome from MS data and Modeling. Related to FIG. 3 and 4.

| | Theoretical | Total protein | ExtraiJectosome | Intra-gectosome | VSV-G-GFP11 | Cre-GFP1-10 | BlaM |
|---|---|---|---|---|---|---|---|
| | lumen volume | volume | protein volume | protein volume | proteinvolume | proteinvolume | protein volume |
| Each Gectosome | (nm³) | (nm³) | (nm³) | (nm³) | (nm³) | (nm³) | (nm³) |
| VSV-G-GFP11/ | 3.43E+06 | 1.35E+06 | 5.63E+05 | 7.87E+05 | 5.45E+05 | 0.00E+00 | 6.80E+01 |
| BlaM | | | | | | | |
| VSV-G-GFP11/ | 2.81E+06 | 1.12E+06 | 5.59E+05 | 5.65E+05 | 5.45E+05 | 5.47E+04 | 0.00E+00 |
| Cre-GFP1-10/BlaM | | | | | | | |

SUPPLEMENTAL TABLE 4
DNA sequencing of the colonies containing the PCSK9 amplified region from
mouse livers edited by VSV-G-GF P-SaCas9-s gPCSK9 Related to FIG. 7.

| | Name of | Expected | Product | | |
|---|---|---|---|---|---|
| PCR Source | TA-Colony | Size | Size | Indel | Reference |
| Template: | Liver1-1 | 919 bp | 919 bp | WT | |
| Mouse livers edited by | Liver1-2 | 919 bp | 919 bp | 1 bp missense mutation | FIG. S7E |
| VSV-G-SaCas9- | Liver1-3 | 919 bp | 910 bp | 9 bp deletion | FIG. S7E |
| sgPCSK9 Gectosomes. | Liver1-4 | 919 bp | 286 bp | 633 bp deletion | FIG. S7F |
| | Liver1-5 | 919 bp | 919 bp | WT | |
| Primers: | Liver1-6 | 919 bp | 919 bp | WT | |
| PCSK9-F and PCSK9-R | Liver1-7 | 919 bp | 919 bp | WT | |
| | Liver1-8 | 919 bp | 183 bp | 736 bp deletion | FIG. S7F |
| | Liver2-1 | 919 bp | 183 bp | 736 bp deletion | FIG. S7F |
| | Liver2-2 | 919 bp | 919 bp | WT | |
| | Liver2-3 | 919 bp | 919 bp | WT | |
| | Liver2-4 | 919 bp | 910 bp | 9 bp deletion | FIG. S7E |
| | Liver2-5 | 919 bp | 918 bp | 1 bp missense mutation | FIG. S7E |
| | Liver2-7 | 919 bp | 918 bp | 1 bp missense mutation | FIG. S7E |
| | Liver2-8 | 919 bp | 919 bp | WT | |
| | Liver3-1 | 919 bp | 919 bp | WT | |
| | Liver3-2 | 919 bp | 158 bp | 801 bp deletion and 40 bp | FIG. S7F |
| | | | | repeat expansion | |
| | Liver3-3 | 919 bp | 919 bp | WT | |
| | Liver3-4 | 919 bp | 918 bp | 1 bp nonsense mutation | FIG. S7E |
| | Liver3-5 | 919 bp | 919 bp | WT | |
| | Liver3-6 | 919 bp | 917 bp | 1 bp deletion | FIG. S7E |
| | Liver3-7 | 919 bp | 917 bp | 1 bp missense mutation | FIG. S7E |

TABLE 1-1
Results of purification of VSV-G-sfGFP gectosomes from 293T cell culture supernatant.

| | | | | Total Protein | Total | VSV-G | Specific | Yield of | Purification |
|---|---|---|---|---|---|---|---|---|---|
| | | Particle | Volume | Concentration | Protein | Amount | Activity | Total Protein | Factor |
| Purification Method | | (×10⁷) | (μl) | (ng/μl) | (ng) | (ng) | (%) | (%) | (Fold) |
| 1 | 2K Spin Supernatant | 9.45 | 10 | 6880 | 68800 | 613.73 | 0.89 | | |
| 2 | 100K Spin Supernatant | 9.45 | 10 | 18 [symbol] .33 | 1893.3 | 405.73 | 21.43 | 2.75 | 24.08 |
| 3 | Flow cytometry Sort | 9.45 | 94. [symbol] | 42 | 3 [symbol] 69 | 2 [symbol] 6954.1 [symbol] 69 | 64.3 [symbol] | [symbol] 69.77 | 72.30 |

[symbol]

indicates data missing or illegible when filed

TABLE 2-1
Results of purification of BlaM-vpr-GFP encapsulated in gectosomes from cell culture supernatant.

| | | | | Total Protein | Total | BlaM | Specific | Yield of | Purification |
|---|---|---|---|---|---|---|---|---|---|
| | | Particle | Volume | Concentration | Protein | Amount | Activity | Total Protein | Factor |
| Purification Method | | (×10⁷) | (μl) | (ng/μl) | (ng) | (ng) | (%) | (%) | (Fold) |
| 1 | 2K Spin Supernatant | 14 | 30 | 2323.64 | [symbol] 9709.1 | 4.0 [symbol] | 0.00 [symbol] | | |
| 2 | 100K Spin Supernatant | 14 | 30 | 13.5 | 4 [symbol] 694.545 | 0. [symbol] 695 | 0.2 | 0.58 | 33.3 |
| 3 | Flow cytometry Sort | 14 | 3 [symbol] 0 | [symbol] .5 [symbol] | 4 [symbol] 699.77 | 1 [symbol] .1 [symbol] | 2.8 | 0.67 | 4 [symbol] .67 |

[symbol]

indicates data missing or illegible when filed

### REFERENCES

[1] Abudayyeh, O. O., Gootenberg, J. S., Essletzbichler, P., Han, S., Joung, J., Belanto, J. J., Verdine, V., Cox, D. B. T., Kellner, M. J., Regev, A., et al. (2017). RNA targeting with CRISPR-Cas13. Nature 550, 280-284.
[2] Ahi, Y. S., Bangari, D. S., and Mittal, S. K. (2011). Adenoviral vector immunity: its implications and circumvention strategies. Curr. Gene Ther. 11, 307-320.
[3 Al-Nedawi, K., Meehan, B., Micallef, J., Lhotak, V., May, L., Guha, A., and Rak, J. (2008). Intercellular transfer of the oncogenic receptor EGFRvIII by microvesicles derived from tumour cells. Nat. Cell Biol. 10, 619.
[4] Al-Nedawi, K., Meehan, B., Kerbel, R. S., Allison, A. C., and Rak, J. (2009). Endothelial expression of autocrine VEGF upon the uptake of tumor-derived microvesicles containing oncogenic EGFR. Proc. Natl. Acad. Sci. 106, 3794-3799.
[5] Barclay, A. N., and Van den Berg, T. K. (2014). The interaction between signal regulatory protein alpha (SIRPα) and CD47: structure, function, and therapeutic target. Annu. Rev. Immunol. 32, 25-50.
[6] Cabantous, S., Terwilliger, T. C., and Waldo, G. S. (2005). Protein tagging and detection with engineered self-assembling fragments of green fluorescent protein. Nat. Biotechnol. 23, 102-107.
[7] Capecchi, M. (1989). Altering the genome by homologous recombination. Science 244, 1288-1292.
[8] Cavrois, M., de Noronha, C., and Greene, W. C. (2002). A sensitive and specific enzyme-based assay detecting HIV-1 virion fusion in primary T lymphocytes. Nat. Biotechnol. 20, 1151-1154.
[9] Chao, M. P., Weissman, I. L., and Majeti, R. (2012). The CD47-SIRPα pathway in cancer immune evasion and potential therapeutic implications. Curr. Opin. Immunol. 24, 225-232.
[10] Charlesworth, C. T., Deshpande, P. S., Dever, D. P., Camarena, J., Lemgart, V. T., Cromer, M. K., Vakulskas, C. A., Collingwood, M. A., Zhang, L., Bode, N. M., et al. (2019). Identification of preexisting adaptive immunity to Cas9 proteins in humans. Nat. Med. 25, 249-254.
[11] Chevillet, J. R., Kang, Q., Ruf, I. K., Briggs, H. A., Vojtech, L. N., Hughes, S. M., Cheng, H. H., Arroyo, J. D., Meredith, E. K., Gallichotte, E. N., et al. (2014). Quantitative and stoichiometric analysis of the microRNA content of exosomes. Proc. Natl. Acad. Sci. 111, 14888-14893.
[12] Clarke, D. C., and Liu, X. (2010). Measuring the absolute abundance of the smad transcription factors using quantitative immunoblotting.
[12] Clarke, D. C., Betterton, M. D., and Liu, X. (2006). Systems theory of Smad signalling. IEE Proc Syst Biol 153, 412-424.
[13] Cocucci, E., and Meldolesi, J. (2015). Ectosomes and exosomes: shedding the confusion between extracellular vesicles. Trends Cell Biol. 25, 364-372.
[14] Cox, D. B. T., Gootenberg, J. S., Abudayyeh, O. O., Franklin, B., Kellner, M. J., Joung, J., and Zhang, F. (2017). RNA editing with CRISPR-Cas13. 10.
[15] Deregibus, M. C., Cantaluppi, V., Calogero, R., Lo Iacono, M., Tetta, C., Biancone, L., Bruno, S., Bussolati, B., and Camussi, G. (2007). Endothelial progenitor cell derived microvesicles activate an angiogenic program in endothelial cells by a horizontal transfer of mRNA. Blood 110, 2440-2448.
[16] Doudna, J. A., and Charpentier, E. (2014). The new frontier of genome engineering with CRISPR-Cas9. Science 346, 1258096-1258096.
[17] Follenzi, A., Santambrogio, L., and Annoni, A. (2007). Immune responses to lentiviral vectors. Curr. Gene Ther. 7, 306-315.
[18] Fu, A., Tang, R, Hardie, J., Farkas, M. E., and Rotello, V. M. (2014). Promises and pitfalls of intracellular delivery of proteins. Bioconjug. Chem. 25, 1602-1608.
[19] Gallardo, H. F., Tan, C., Ory, D., and Sadelain, M. Recombinant Retroviruses Pseudotyped With the Vesicular Stomatitis Virus G Glycoprotein Mediate Both Stable Gene Transfer and Pseudotransduction in Human Peripheral Blood Lymphocytes. 7.
[20] Hammond, S. M. (2001). Argonaute2, a Link Between Genetic and Biochemical Analyses ofRNAi. Science 293, 1146-1150.
[21] Haraszti, R. A., Didiot, M. -C., Sapp, E., Leszyk, J., Shaffer, S. A., Rockwell, H. E., Gao, F., Narain, N. R., DiFiglia, M., Kiebish, M. A., et al. (2016). High-resolution proteomic and lipidomic analysis of exosomes and microvesicles from different cell sources. J Extracell Vesicles 5, 32570.
[22] Hirschberg, K., Miller, C. M., Ellenberg, J., Presley, J. F., Siggia, E. D., Phair, R. D., and Lippincott-Schwartz, J. (1998). Kinetic analysis of secretory protein traffic and characterization of golgi to plasma membrane transport intermediates in living cells. J. Cell Biol. 143, 1485-1503.
[23] Hurley, J. H., Boura, E., Carlson, L. -A., and Różycki, B. (2010). Membrane Budding. Cell 143, 875-887.
[24] Jeppesen, D. K., Fenix, A. M., Franklin, J. L., Higginbotham, J. N., Zhang, Q., Zimmerman, L. J., Liebler, D. C., Ping, J., Liu, Q., Evans, R., et al. (2019). Reassessment of Exosome Composition. Cell 177, 428-445.e18.
[25] Kaczmarek, J. C., Kowalski, P. S., and Anderson, D. G. (2017). Advances in the delivery of RNA therapeutics: from concept to clinical reality. Genome Med. 9, 60.
[26] Kamerkar, S., LeBleu, V. S., Sugimoto, H., Yang, S., Ruivo, C. F., Melo, S. A., Lee, J. J., and Kalluri, R. (2017). Exosomes facilitate therapeutic targeting of oncogenic KRAS in pancreatic cancer. Nature 546, 498-503.
[27] Kanada, M., Bachmann, M. H., Hardy, J. W., Frimannson, D. O., Bronsart, L., Wang, A., Sylvester, M. D., Schmidt, T. L., Kaspar, R. L., Butte, M. J., et al. (2015). Differential fates of biomolecules delivered to target cells via extracellular vesicles. Proc. Natl. Acad. Sci. 112, E1433- E1442.
[28] Kowal, J., Arras, G., Colombo, M, Jouve, M., Morath, J. P., Primdal-Bengtson, B., Dingli, F., Loew, D , Tkach, M., and Théry, C. (2016). Proteomic comparison defines novel markers to characterize heterogeneous populations of extracellular vesicle subtypes. Proc. Natl. Acad. Sci. 113, E968-E977.
[29] Levin, A. A. (2019). Treating Disease at the RNA Level with Oligonucleotides. N. Lngl. J. Med. 380, 57-70.
[30] Liu, M. -L., Winther, B. L., and Kay, M. A. (1996). Pseudotransduction of Hepatocytes by Using Concentrated Pseudotyped Vesicular Stomatitis Virus G Glycoprotein (VSV-G)-Moloney Murine Leukemia Virus-Derived Retrovirus Vectors: Comparison of VSV-G and Amphotropic Vectors for Hepatic Gene Transfer. J VIROL 70, 6.
[31] Lodish, H. F., and Weiss, R. A. (1979). Selective Isolation of Mutants of Vesicular Stomatitis Virus Defective in Production of the Viral Glycoprotein. J VIROL 30, 13.
[32] Maeder, M. L., and Gersbach, C. A. (2016). Genome-editing Technologies for Gene and Cell Therapy. Mol. Ther. 24, 430-446.
[33] Mangeot, P. -E., Dollet, S., Girard, M., Ciancia, C., Joly, S., Peschanski, M., and l.otteau, V. (2011). Protein Transfer Into Human Cells by VSV-G-induced Nanovesicles. Mol. Ther. 19, 1656-1666.
[34] Mathieu, M., Martin-Jaular, L., Lavieu, G., and Théry, C. (2019). Specificities of secretion and uptake of exosomes and other extracellular vesicles for cell-to-cell communication. Nat. Cell Biol. 21, 9-17.
[35] Meister, G., and Tuschl, T. (2004). Mechanisms of gene silencing by double-stranded RNA. Nature 431, 343-349.
[36] Messenger, S. W, Woo, S. S., Sun, Z., and Martin, T. F. J. (2018). A Ca 2+-stimulated exosome release pathway in cancer cells is regulated by Munc13-4 J. Cell Biol. jcb.201710132.
[37] Narendra, D. P., Jin, S. M., Tanaka, A., Suen, D. -F., Gautier, C. A., Shen, J., Cookson, M. R., and Youle, R. J. (2010). PINK1 is selectively stabilized on impaired mitochondria to ECtivate Parkin PLoS Biol 8.
van Niel, G., D'Angelo, G., and Raposo, G. (2018). Shedding light on the cell biology of extracellular vesicles. Nat. Rev. Mol. Cell Biol. 19, 213-228.
[38] Pan, D., Gunther, R., Duan, W., Wendell, S., Kaemmerer, W., Kafri, T., Verma, I. M., and Whitley, C. B. (2002). Biodistribution and Toxicity Studies of VSVG-Pseudotyped Lentiviral Vector after Intravenous Administration in Mice with the Observation of in Vivo Transduction of Bone Marrow. Mol. Ther. 6, 19-29.
[39] Ran, F. A., Cong, L., Yan, W. X., Scott, D. A., Gootenberg, J. S., Kriz, A. J., Zetsche, B., S halem, O., Wu, X., Makarova, K. S., et al. (2015). In vivo genome editing using Staphylococcus aureus Cas9. Nature 520, 186-191.
[40] Raposo, G., and Stoorvogel, W. (2013). Extracellular vesicles: Exosomes, microvesicles, and friends. J Cell Biol 200, 373-383.
[41] Rolls, M. M., Webster, P., Balba, N. H., and Rose, J. K. (1994). Novel infectious particles generated by expression of the vesicular stomatitis virus glycoprotein from a self-replicating RNA. Cell 79, 497-506.
[42] Rose, N. F., Buonocore, L., Schell, J. B., Chattopadhyay, A., Bahl, K., Liu, X., and Rose. J. K. (2014). In vitro evolution of high-titer, virus-like vesicles containing a single structural protein. Proc. Natl. Acad. Sci. 111, 16866-16871.
[43] Shalem, O., Sanjana, N. E., and Zhang, F. (2015). High-throughput functional genomics using CRISPR-Cas9. Nat. Rev. Genet. 16, 299-311.
[44] Skog, J., Wurdinger, T., van Rijn, S., Meijer, D. H., Gainche, L., Curry Jr., W. T., Carter, B. S., Krichevsky, A. M., and Breakefield, X. O. (2008). Glioblastoma microvesicles transport RNA and proteins that promote tumour growth and provide diagnostic biomarkers. Nat. Cell Biol. 10, 1470.
[45] Sockolosky, J. T., Dougan, M., Ingram, J. R., Ho, C. C. M., Kauke, M. J., Almo, S. C., Ploegh, H. L., and Garcia, K. C. (2016). Durable antitumor responses to CD47 blockade require adaptive immune stimulation. Proc. Natl. Acad. Sci. U.S.A. 113, E2646-2654.
[46] Tkach, M., and Théry, C. (2016). Communication by Extracellular Vesicles: Where We Are and Where We Need to Go. Cell 164, 1226-1232.
[47] Valadi, H., Ekström, K, Bossios, A., Sjöstrand, M., Lee, J. J., and Lötvall, J. O. (2007). Exosome-mediated transfer of mRNAs and microRNAs is a novel mechanism of genetic exchange between cells. Nat Cell Biol 9, 654-659.
[48] Votteler, J., and Sundquist, W. I. (2013). Virus Budding and the ESCRT Pathway. Cell Host Microbe 14, 232-241.
[49] Votteler, J., Ogohara, C., Yi, S., Hsia, Y., Nattermann, U., Belnap, D. M., King, N. P., and Sundquist, W. I. (2016). Designed proteins induce the formation of nanocage-containing extracellular vesicles. Nature 540, 292-295.
[50] Weissenhorn, W., Hinz, A., and Gaudin, Y. (2007). Virus membrane fusion. FEBS Lett. 581,2150-2155.
[51] Weissenhorn, W., Poudevigne, E., Effantin, G, and Bassereau, P (2013). How to get out: ssRNA enveloped viruses and membrane fission. Curr. Opin. Virol. 3, 159-167.
[52] Wittrup, A., Ai, A., Liu, X., Hamar, P., Trifonova, R. , Charisse, K. , Manoharan, M., Kirchhausen, T., and Lieberman, J. (2015). Visualizing lipid-formulated siRNA release from endosomes and target gene knockdown. Nat. Biotechnol. 33, 870.
[53] Zhang, C., Lee, S., Peng, Y., Bunker, E., Giaime, E., Shen, J., Zhou, Z., and Liu, X. (2014a). PINK1 triggers autocatalytic activation of parkin to specify cell fate decisions. Curr. Biol. 24,1854-1865.
[54] Zhang, C., Lee, S., Peng, Y., Bunker, E., Giaime, E., Shen, J., Zhou, Z., and Liu, X. (2014b). PINK1 triggers autocatalytic activation of parkin to specify cell fate decisions. Curr Biol 24, 1854-1865.
[55] Zhang, C., Liu, Z., Bunker, E., Ramirez, A., Lee, S., Peng, Y., Tan, A. C., Eckhardt, S.G., Chapnick, D. A., and Liu, X. (2017). Sorafenib targets the mitochondrial electron transport chain complexes and ATP synthase to activate the PINK 1-Parkin pathway and modulate cellular drug response. J Biol Chem 292, 15105-15120.
[56] Zhang, Y., Chopp, M., Meng, Y , Katakowski, M., Xin, H., Mahmood, A., and Xiong, Y. (2015). Effect of exosomes derived from multipluripotent mesenchymal stromal cells on functional recovery and neurovascular plasticity in rats after traumatic brain injury. J Neurosurg 122, 856-867.
[57] Zomer, A., Maynard, C., Verweij, F. J., Kamermans, A., Schäfer, R., Beerling, E., Schiffelers, R. M., de Wit, E., Berenguer, J., Ellenbroek, S. I. J., et al. (2015). In Vivo Imaging Reveals Extracellular Vesicle-Mediated Phenocopying of Metastatic Behavior. Cell 161, 1046- 1057.
[58] U.S. patent application Ser. No. 13/505,506.
[59] U.S. Pat. No. 9,593,356.

SEQUENCE LISTING
Amino Acid
VSV-G
Vesicular Stomatitis Virus
SEQ ID NO. 1
MKCLLYLAFLFIGVNCKFTIVFPHNQKGNWKNVPSNYHYCPSSSDLNWH
NDLIGTAIQVKMPKSHKAIQADGWMCHASKWVTTCDFRWYGPKYITQSI
RSFTPSVEQCKESIEQTKQGTWLNPGFPPQSCGYATVTDAEAVIVQVTP
HHVLVDEYTGEWVDSQFINGKCSNYICPTVHNSTTWHSDYKVKGLCDSN
LISMDITFFSEDGELSSLGKEGTGFRSNYFAYETGGKACKMQYCKHWGV
RLPSGVWFEMADKDLFAAARFPECPEGSSISAPSQTSVDVSLIQDVERI
LDYSLCQETWSKIRAGLPISPVDLSYLAPKNPGTGPAFTIINGTLKYFE
TRYIRVDIAAPILSRMVGMISGTTTERELWDDWAPYEDVEIGPNGVLRT
SSGYKFPLYMIGHGMLDSDLHLSSKAQVFEHPHIQDAASQLPDDESLFF
GDTGLSKNPIELVEGWFSSWKSSIASFFFIIGLIIGLFLVLRVGIHLCI
KLKHTKKRQIYTDIEMNRLGK
Amino Acid
p6^{GAG}
Human immunodeficiency virus type 1 (HIV-1)
SEQ ID NO. 2
LQSRPEPTAPPEESFRSGVETTTPPQKQEPIDKELYPLTSLRSLFGNDP
SSQ
DNA
PINK1, Forward
Artificial
SEQ ID NO. 3
CACCGCCTGGAGGTGACAAAGAGCA
DNA
PINK1, Reverse
Artificial
SEQ ID NO. 4
AAACTGCTCTTTGTCACCTCCAGGC
DNA
GAPDH Forward
Artificial
SEQ ID NO. 5
GAAGGTGAAGGTCGGAGT
DNA
GAPDH Reverse
Artificial
SEQ ID NO. 6
GAAGATGGTGATGGGATTTC
DNA
PINK1Ex1
Artificial
SEQ ID NO. 7
CGCTGCTGCTGCGCTTCA
DNA
PINK1Ex3
Artificial
SEQ ID NO. 8
CTGCTCCATACTCCCCAGCC
DNA
PINK1Int2
Artificial
SEQ ID NO. 9
GTCTCCATAATCAGACACCT
DNA
PINK1Int3
Artificial
SEQ ID NO. 10
GGATGGTGAACTAACCAATC
DNA
mPCSK9, Forward
Artificial
SEQ ID NO. 11
GATGCCACTTTACTTCGGAGGA
DNA
mPCSK9, Reverse
Artificial
SEQ ID NO. 12
AGGAGGATTGGAGTGGGGATTA

What is claimed is:

1. A method of selectively delivering a target molecule to a recipient cell comprising the steps of:
transfecting a donor cell to heterologously express a two component delivery system comprising:
a protein capable of being incorporated into the membrane of an extracellular vesicle (EV) coupled with a first component of a split complement system;
a second component of said split complement system configured to be coupled with a molecule;
optionally a protein, or protein fragment configured to increase delivery of said target molecule;
anchoring said target molecule to a membrane capable of forming an EV by reconstituting said split complement system; and
encapsulating said target molecule and said reconstituted split complement system in an EV formed from said donor cell.

2. The method of claim 1, further comprising the step of fusing said EV formed from said donor cell with a recipient cell.

3. The method of claim 2, further comprising the step of releasing said target molecule from said EV formed from said donor cell into said recipient cell.

4. The method of claim 3, further comprising the step of administering a therapeutically effective amount of said target molecule to a subject in need thereof.

5. The method of claim 1, wherein said protein capable of being incorporated into the membrane of an EV comprises a fusogenic protein capable of being incorporated into the membrane of an EV.

6. The method of claim 5, wherein said EV comprises an ectosome.

7. The method of claim 5, wherein the fusogenic protein capable of being incorporated into the membrane of an EV comprises a fusogenic protein selected from the group consisting of: a vesicular stomatitis virus G (VSV-G) viral fusion protein, a protein according to SEQ ID NO. 1, and a fusogenic protein capable having at least 80% sequence identity with SEQ ID NO. 1.

8. The method of claim 7, wherein said protein configured to increase delivery of said target molecule comprises a protein or protein fragment selected from the group consisting of: a protein or protein fragment having a Gag peptide motif, and a p6^{Gag} peptide according to SEQ ID NO. 2.

9. The method of claim 7, wherein said VSV-G protein comprises a VSV-G protein having an additional binding motif selected from the group consisting of: a DNA binding motif; an RNA binding motif; a protein binding motif, and ligand binding motif.

10. The method of claim 9, wherein said VSV-G protein having an additional binding motif comprises a VSV-G protein coupled with a tag.

11. The method of claim 7, wherein said VSV-G protein comprises a fusion deficient VSV-G mutant protein.

12. The method of claim 1, wherein said first component of said split complement system comprises a GFP11 peptide and said second component of said split complement system comprises a GFP1-10 peptide, that when reconstituted form an active green fluorescent protein (GFP).

13. The method of claim 1, wherein said split complement system comprises a split complement system selected from the group consisting of: a split GFP system; a NanoBiT split ubiquitin system; a split beta-gal system; a split luciferase system; a split mCherry system; a split FRET system; and a split biotin system.

14. The method of claim 1 wherein said target molecule comprises a target molecule selected from the group consisting of: a protein, a protein fragment; a therapeutic protein; a cellular reprogramming protein; a labeled protein; a peptide aptamer; an antibody; an antibody fragment; tumor specific antigen peptide; a genome editing enzyme; an antigen; an oligonucleotide; a meganucleases; a nucleic acid; a DNA molecule; an RNA molecule; an RNAi molecule; a protein involved in the RNA-induced silencing complex (RISC); a therapeutic compound; a nanoparticle; a ligand; and a prodrug.

15. The method of claim 14 wherein said genome editing enzyme comprises a genome editing enzyme selected from the group consisting of: a nuclease; Cas9; dCas9; SaCas9; dSaCas9; LwaCas13; Cas13; C2c1; C2C3; C2c2; Cfp1; CasX; base editors constructed by dCas9 fusion to a cytidine deaminase protein, CRISPRi; CRISPRa; CRISPRX; CRISPR-STOP; a TALEN nuclease; and a Zinc-Finger nuclease; and a CRE recombinase.

16. The method of claim 15, further comprising the step of introducing to donor cell a sgRNA directed to a target gene, or transfecting said donor cell to heterologously express a sgRNA directed to a target gene.

17. The method of claim 16, wherein said sgRNA directed to a target gene binds with at least genome editing enzyme and is encapsulated in said EV.

18. The method of claims 17 and 9, wherein said sgRNA directed to a target gene is coupled with a VSV-G protein having an RNA binding motif.

19. The method of claim 14 wherein the protein involved in the RISC comprises AGO2.

20. The method of claim 19, further comprising the step of introducing to donor cell an RNAi molecule configured to downregulate expression of a target gene, or transfecting said donor cell to heterologously co-expressing an RNAi molecule configured to downregulate expression of a target gene.

21. The method of claim 20, wherein said a RNAi molecule configured to downregulate expression of a target gene binds with a protein involved in the RISC and is encapsulated in said EV.

22. The method of claim 21, wherein said RNAi molecule comprises an RNAi molecule selected from the group consisting of: a dsRNA molecule; an siRNA molecule; a miRNA molecule; a lincRNAs molecules and a shRNA molecule.

23. The method of claim 1, wherein said reconstituted split complement system emits a detectable signal.

24. The method of claim 23, further comprising the step of isolating one or more EVs based on said detectable signal generated by said reconstituted split complement system.

25. The method of claim 1, further comprising the step of transfecting said donor cell to overexpress one or more proteins that disrupt clearance of said EV by macrophages or dendritic cells, or alternatively transfecting said donor cell to overexpress one or more proteins that promoted clearance of said EV by macrophages or dendritic cells.

26. The method of claim 25, wherein said step of transfecting said donor cell to overexpress one or more proteins that disrupt macrophage clearance of said EV comprises the step of transfecting said donor cell to overexpress CD47.

27. The method of claim 1, wherein said step of transfecting said donor cell to overexpress one or more proteins that disrupt macrophage clearance of said EV comprises the step of transfecting said donor cell to overexpress CD47, or alternatively transfecting said donor cell to overexpress one or more proteins that promoted clearance of said EV by macrophages or dendritic cells comprises step of transfecting said donor cell to overexpress an anti-CD47 nanobody.

28. The method of claim of claim 1, performed in vitro, ex vivo or in vivo.

29. A method of selectively delivering a target ligand to a recipient cell comprising the steps of:
transfecting a donor cell to heterologously express a two component delivery system comprising:
a protein capable of being incorporated into the membrane of an extracellular vesicle (EV) coupled with a first component of a split complement system and optionally configured to be coupled with at least one target ligand;
a second component of said split complement system configured to be coupled with at least one target ligand;
optionally a protein, or protein fragment configured to increase delivery of said target ligand;
anchoring said at least one target ligand to a membrane capable of forming an EV by reconstituting said split complement system; and
encapsulating said target ligand and said reconstituted split complement system in an EV formed from said donor cell.

30. The method of claim 29, wherein said membrane-bound protein comprises a membrane-bound protein selected from the group consisting of: a vesicular stomatitis virus G (VSV-G) viral fusion protein a protein according to SEQ ID NO. 1, or a fusogenic protein capable having at least 80% sequence identity with SEQ ID NO. 1, and wherein said protein configured to increase delivery of said target molecule comprises a protein or protein fragment selected from the group consisting of: a protein or protein fragment having a Gag peptide motif, and a p6^{Gag} peptide according to SEQ ID NO. 2.

31. The method of claim 29, wherein said split complement system comprises a split complement system selected from the group consisting of: a split GFP system; a NanoBiT split ubiquitin system; a split beta-gal system; a split luciferase system; a split mCherry system; a split FRET system; and a split biotin system.

32. The method of claim 29, wherein said target ligand comprises a target molecule selected from the group consisting of: a protein, a protein fragment; a therapeutic protein; a cellular reprogramming protein; a labeled protein; a peptide aptamer; an antibody; an antibody fragment; tumor specific antigen peptide; a genome editing enzyme; an antigen; an oligonucleotide; a meganuclease; a nucleic acid; a DNA molecule; an RNA molecule; an RNAi molecule; a protein involved in the RNA-induced silencing complex (RISC); a therapeutic compound; a nanoparticle; a ligand; and a prodrug.

33. The method of claim 32, and further comprising a nucleotide configured to be coupled with said target ligand, or said membrane-bound protein, or said second component of said split complement system.

34. The method of claim 33, wherein a nucleotide comprises a nucleotide selected from the group consisting of: a sgRNA; an RNAi molecule; and a DNA molecule.

35. A method of transiently or stably transfecting a recipient cell through a programmable extracellular vesicle, comprising the steps of:
transfecting a donor cell to heterologously express a two component delivery system comprising:
a viral fusion protein G from Vesicular Stomatitis Virus (VSV-G) incorporated into the membrane of an extracellular vesicle (EV) coupled with a first component of a GFP split complement system and optionally configured to be coupled with at least one target ligand;
a second component of said GFP split complement system configured to be coupled with at least one target ligand;
a protein or protein fragment having a Gag peptide motif;
anchoring the at least one target ligand to a membrane capable of forming an EV by reconstituting said split complement system; and
forming one or more EVs from said donor cell encapsulating said at least one target ligand and said reconstituted split complement system.

36. The method of claim 35, wherein said split complement system comprises a split complement system selected from the group consisting of: a split GFP system; a NanoBiT split ubiquitin system; a split beta-gal system; a split luciferase system; a split mCherry system; a split FRET system; and a split biotin system.

37. The method of claim 35, wherein said target ligand comprises a target molecule selected from the group consisting of: a protein, a protein fragment; a therapeutic protein; a cellular reprogramming protein; a labeled protein; a peptide aptamer; an antibody; an antibody fragment; tumor specific antigen peptide; a genome editing enzyme; an antigen; an oligonucleotide; a meganucleases; a nucleic acid; a DNA molecule; an RNA molecule; an RNAi molecule; a protein involved in the RNA-induced silencing complex (RISC); a therapeutic compound; a nanoparticle; a ligand; and a prodrug.

38. The method of claim 37, and further comprising a nucleotide configured to be coupled with said target ligand, or said membrane-bound protein, or said second component of said split complement system.

39. The method of claim 35, wherein a nucleotide comprises a nucleotide selected from the group consisting of: a sgRNA; an RNAi molecule; and a DNA molecule.

40. A method of selectively delivering a target ligand to a recipient cell comprising the steps of:
transfecting a donor cell to heterologously express a protein capable of being incorporated into the membrane of an extracellular vesicle (EV) and further being configured to be coupled with at least one target ligand; and
optionally a protein, or protein fragment configured to increase delivery of said target ligand;
forming one or more EVs from said donor cell encapsulating said target ligand.

41. The method of claim 40, wherein said membrane-bound protein comprises a membrane-bound protein selected from the group consisting of: a vesicular stomatitis virus G (VSV-G) viral fusion protein a protein according to SEQ ID NO. 1, or a fusogenic protein capable having at least 80% sequence identity with SEQ ID NO. 1, and wherein said protein configured to increase delivery of said target molecule comprises a protein or protein fragment selected from the group consisting of: a protein or protein fragment having a Gag peptide motif, and a p6^{Gag} peptide according to SEQ ID NO. 2.

42. The method of claim 40, and further comprising a tag coupled with said protein capable of being incorporated into the membrane of an EV.

43. The method of claim 40, wherein said target ligand comprises a target molecule selected from the group consisting of: a protein, a protein fragment; a therapeutic protein; a cellular reprogramming protein; a labeled protein; a peptide aptamer; an antibody; an antibody fragment; tumor specific antigen peptide; a genome editing enzyme; an antigen; an oligonucleotide; a meganuclease; a nucleic acid; a DNA molecule; an RNA molecule; an RNAi molecule; a protein involved in the RNA-induced silencing complex (RISC); a therapeutic compound; a nanoparticle; a ligand; and a prodrug.

44. The method of claim 43, and further comprising a nucleotide configured to be coupled with said target ligand, or protein capable of being incorporated into the membrane, or encapsulated within said one or more EVs.

45. The method of claim 44, wherein a nucleotide comprises a nucleotide selected from the group consisting of: a sgRNA; an RNAi molecule; and a DNA molecule.

46. A composition comprising:
a gectosome having membrane bound vesicular stomatitis virus G (VSV-G) viral fusion protein coupled with a first component of a split complement system, and a second component of said split complement system, wherein said membrane-bound protein or said second component of said split complement system are configured to be coupled with at least one target molecule; and
a p6Gag peptide.

47. The composition of claim 46, wherein said split complement system comprises a split complement system selected from the group consisting of: a split GFP system; a NanoBiT split ubiquitin system; a split beta-gal system; a split luciferase system; a split mCherry system; a split FRET system; and a split biotin system.

48. The composition of claim 46, wherein said target molecule comprises a target molecule selected from the group consisting of: a protein, a protein fragment; a therapeutic protein; a cellular reprogramming protein; a labeled protein; a peptide aptamer; an antibody; an antibody fragment; tumor specific antigen peptide; a genome editing enzyme; an antigen; an oligonucleotide; a meganuclease; a nucleic acid; a DNA molecule; an RNA molecule; an RNAi molecule; a protein involved in the RNA-induced silencing complex (RISC); a therapeutic compound; a nanoparticle; a ligand; and a prodrug.

49. The composition of claim 48, and further comprising a nucleotide configured to be coupled with said target molecule, or said VSV-G viral fusion protein, or said first or second component of said split complement system or encapsulated within said gectosome.

50. The composition of claim 49, wherein a nucleotide comprises a nucleotide selected from the group consisting of: a sgRNA; an RNAi molecule; and a DNA molecule.

51. A composition comprising:
an extracellular vesicle (EV) having:
a membrane-bound protein coupled with a first component of a split complement system and further configured to be capable of being coupled with a target molecule;
a second component of said split complement system configured to be capable of being coupled with at least one target molecule; and
optionally a protein, or protein fragment configured to increase delivery of said target molecule.

52. The composition of claim 51, wherein said membrane-bound protein comprises a vesicular stomatitis virus G (VSV-G) viral fusion protein, a protein according to SEQ ID NO. 1, and a fusogenic protein capable having at least 80% sequence identity with SEQ ID NO. 1, and wherein said protein configured to increase delivery of said target molecule comprises a protein or protein fragment selected from the group consisting of: a protein or protein fragment having a Gag peptide motif, and a p6^{Gag} peptide according to SEQ ID NO. 2.

53. The composition of claim 51, wherein said split complement system comprises a split complement system selected from the group consisting of: a split GFP system; a NanoBiT split ubiquitin system; a split beta-gal system; a split luciferase system; a split mCherry system; a split FRET system; and a split biotin system.

54. The composition of claim 51, wherein said target molecule comprises a target molecule selected from the group consisting of: a protein, a protein fragment; a therapeutic protein; a cellular reprogramming protein; a labeled protein; a peptide aptamer; an antibody; an antibody fragment; tumor specific antigen peptide; a genome editing enzyme; an antigen; an oligonucleotide; a meganuclease; a nucleic acid; a DNA molecule; an RNA molecule; an RNAi molecule; a protein involved in the RNA-induced silencing complex (RISC); a therapeutic compound; a nanoparticle; a ligand; and a prodrug.

55. The composition of claim 54, and further comprising a nucleotide configured to be coupled with said target molecule, or said membrane-bound protein, or said second component of said split complement system, or encapsulated within said EV.

56. The composition of claim 51, wherein a nucleotide configured to be coupled with said target molecules or said membrane-bound protein comprises a nucleotide selected from the group consisting of: a sgRNA; an RNAi molecule; and a DNA molecule.

57. A method of amplifying an immune response in a subject comprising the steps of:
transfecting a donor cell to heterologously express:
a fusion deficient fusogenic protein coupled with a first component of a split complement system;
a second component of a split complement system fused with an antibody peptide or a tumor specific antigen peptide;
antibody peptide or a tumor specific antigen peptide to a membrane an antibody peptide or a tumor specific antigen peptide to a membrane;
anchoring said antibody peptide or a tumor specific antigen peptide to a membrane capable of forming an EV by reconstituting said split complement system;
forming one or more EVs from said donor cell wherein the antibody peptide or a tumor specific antigen peptide is presented on the surface of said one or more EVs;
isolating said one or more EVs; and
administering a therapeutically effective amount of said isolated EVs to a subject in need thereof wherein the antibody peptide or tumor specific antigen peptide presented on the surface of said isolated EVs elicit an immune response in said subject.

58. The method of claim 57, wherein said fusion deficient fusogenic protein comprises a fusion deficient VSV-G mutant protein, and wherein said protein configured to increase delivery of said target molecule comprises a protein or protein fragment selected from the group consisting of: a protein or protein fragment having a Gag peptide motif, and a p6^{Gag} peptide according to SEQ ID NO. 2.

59. The method of claim 57, wherein said split complement system comprises a split complement system selected from the group consisting of: a split GFP system; a NanoBiT split ubiquitin system; a split beta-gal system; a split luciferase system; a split mCherry system; a split FRET system; and a split biotin system.

60. The method of claim 57, wherein said first component of said split complement system comprises a GFP11 peptide and said second component of said split complement system comprises a GFP1-10 peptide, that when reconstituted form an active green fluorescent protein (GFP).

61. The method of claim 57, wherein said antibody peptide comprises a bispecific antibody peptide or a fragment thereof.

62. The method of claim 61, wherein said bispecific antibody peptide or a fragment thereof comprises a bispecific antibody peptide selected from the group consisting of: CD3; and EGFR.

63. The method of claim 57, wherein said tumor specific antigen peptide comprises a tumor specific antigen selected from the group consisting of: dopachrome-tautomerase (TRP2), melanocyte protein PMEL (gp100), HPV E6/7, MAGE 1, MAGE 3, NY-ESO, androgen receptor (AR), BCL-1, calprotectin, carcinoembryonic antigen (CEA), EGFRs, epithelial cell adhesion molecule (Ep-CAM), epithelial sialomucin, membrane estrogen receptors (mER), FAP HER2/neu, human high molecular weight melanoma-associated antigen (HMW-MAA), IL-6, MOC-1, MOC-21, MOC-52, melan-A/MART-1, melanoma-associated antigen, mucin, OKT9, progesterone receptor (PGR), prostate specific antigen (PSA), prostate stem cell antigen (PSCA), prostate-specific membrane antigen (PSMA), symaptophysin, VEGFRs, CD19, CD20, CD22, CD30 and CD33.

64. The method of claim 57, wherein said step of isolating one or more EVs comprises the step of isolating one or more EVs based on a detectable signal generated by said reconstituted split complement system.

65. The method of claim 57, wherein said immune response comprises CD8-T cell activation in a subject.

66. The method of claim 57, further comprising the step of transfecting said donor cell to overexpress one or more proteins that disrupt clearance of said EV by macrophages or dendritic cells, or alternatively transfecting said donor cell to overexpress one or more proteins that promoted clearance of said EV by macrophages or dendritic cells

67. The method of claim 66, wherein said step of transfecting said donor cell to overexpress one or more proteins that disrupt macrophage clearance of said EV comprises the step of transfecting said donor cell to overexpress CD47, or alternatively transfecting said donor cell to overexpress one or more proteins that promoted clearance of said EV by macrophages or dendritic cells comprises step of transfecting said donor cell to overexpress an anti-CD47 nanobody.

68. The method of claim of claim 57, performed in vitro, ex vivo or in vivo.

69. A method of amplifying an immune response in a subject comprising the steps of:
transfecting a donor cell to heterologously express a fusion deficient protein capable of being incorporated into the membrane of an extracellular vesicle (EV) and further being configured to be coupled with at least one antibody peptide, or a tumor specific antigen peptide;
forming one or more EVs from said donor cell wherein the antibody peptide or a tumor specific antigen peptide is presented on the surface of said one or more EVs;
isolating said one or more EVs; and
administering a therapeutically effective amount of said isolated EVs to a subject in need thereof wherein the antibody peptide or tumor specific antigen peptide presented on the surface of said isolated EVs elicit an immune response in said subject.

70. The method of claim 69, wherein said fusion deficient protein comprises a fusion deficient VSV-G mutant protein.

71. The method of claim 70, wherein said fusion deficient VSV-G mutant protein comprises a tagged fusion deficient VSV-G mutant protein.

72. The method of claim 71, wherein said step of isolating one or more EVs comprises the step of isolating one or more EVs based on the tag coupled with said fusion deficient VSV-G mutant protein.

73. The method of claim 70, wherein said antibody peptide comprises a bispecific antibody peptide or a fragment thereof.

74. The method of claim 73, wherein said bispecific antibody peptide or a fragment thereof comprises a bispecific antibody peptide selected from the group consisting of: CD3; and EGFR. 69, wherein said tumor specific antigen peptide comprises tumor specific antigen selected from the group consisting of: dopachrome-tautomerase (TRP2), melanocyte protein PMEL (gp100), HPV E6/7, MAGE 1, MAGE 3, NY-ESO, androgen receptor (AR), BCL-1, calprotectin, carcinoembryonic antigen (CEA), EGFRs, epithelial cell adhesion molecule (Ep-CAM), epithelial sialomucin, membrane estrogen receptors (mER), FAP HER2/neu, human high molecular weight melanoma-associated antigen (HMW-MAA), IL-6, MOC-1, MOC-21, MOC-52, melan-A/MART-1, melanoma-associated antigen, mucin, OKT9, progesterone receptor (PGR), prostate specific antigen (PSA), prostate stem cell antigen (PSCA), prostate-specific membrane antigen (PSMA), symaptophysin, VEGFRs, CD19, CD20, CD22, CD30 and CD33.

75. The method of claim 70, wherein said antibody peptide comprises a monoclonal antibody peptide or a fragment thereof.

76. The method of claim 69, wherein said immune response comprises CD8-T cell activation in a subject.

77. The method of claim 69, further comprising the step of transfecting said donor cell to overexpress one or more proteins that disrupt clearance of said EV by macrophages or dendritic cells, or alternatively transfecting said donor cell to overexpress one or more proteins that promoted clearance of said EV by macrophages or dendritic cells.

78. The method of claim 77, wherein said step of transfecting said donor cell to overexpress one or more proteins that disrupt macrophage clearance of said EV comprises the step of transfecting said donor cell to overexpress CD47, or alternatively transfecting said donor cell to overexpress one or more proteins that promoted clearance of said EV by macrophages or dendritic cells comprises step of transfecting said donor cell to overexpress an anti-CD47 nanobody.

79. A composition comprising an EV having a fusion deficient fusogenic protein capable of being incorporated into the membrane of an extracellular vesicle (EV) and further being configured to be coupled with at least one antibody peptide, or a tumor specific antigen peptide.

80. The composition of claim 79, wherein said fusion deficient fusogenic protein comprises a vesicular stomatitis virus G (VSV-G) viral fusion protein.

81. The composition of claim 80, wherein said fusion deficient VSV-G mutant protein comprises a tagged fusion deficient VSV-G mutant protein.

82. The composition of claim 79, wherein said antibody peptide comprises a bispecific antibody peptide or a fragment thereof.

83. The composition of claim 82, wherein said bispecific antibody peptide or a fragment thereof comprises a bispecific antibody peptide selected from the group consisting of: CD3; and EGFR.

84. The composition of claim 79, wherein said tumor specific antigen peptide comprises tumor specific antigen selected from the group consisting of: dopachrome-tautomerase (TRP2), melanocyte protein PMEL (gp100), HPV E6/7, MAGE 1, MAGE 3, NY-ESO, androgen receptor (AR), BCL-1, calprotectin, carcinoembryonic antigen (CEA), EGFRs, epithelial cell adhesion molecule (Ep-CAM), epithelial sialomucin, membrane estrogen receptors (mER), FAP HER2/neu, human high molecular weight melanoma-associated antigen (HMW-MAA), IL-6, MOC-1, MOC-21, MOC-52, melan-A/MART-1, melanoma-associated antigen, mucin, OKT9, progesterone receptor (PGR), prostate specific antigen (PSA), prostate stem cell antigen (PSCA), prostate-specific membrane antigen (PSMA), symaptophysin, VEGFRs, CD19, CD20, CD22, CD30 and CD33.

85. The composition of claim 79, wherein said immune response comprises CD8-T cell activation in a subject.

86. The composition of claim 79, and further comprising one or more proteins that disrupt macrophage or dendritic cell clearance of said EV by, or alternatively further comprising one or more proteins that promotes macrophage or dendritic cell clearance of said EV.

87. The composition of claim 86, and further comprising one or more proteins that disrupt macrophage or dendritic cell clearance of said EV by, or alternatively further comprising one or more proteins that promotes macrophage or dendritic cell clearance of said EV.

88. A composition comprising an EV having a fusion deficient fusogenic protein coupled with a first component of a split complement system, and a second component of said split complement system, wherein said membrane-bound protein and said second component of said split complement system are optionally configured to be coupled with at least one target molecule.

89. The composition of claim 88, wherein said fusion deficient fusogenic protein comprises a vesicular stomatitis virus G (VSV-G) viral fusion protein.

90. The composition of claim 89, wherein said split complement system comprises a split complement system selected from the group consisting of: a split GFP system; a NanoBiT split ubiquitin system; a split beta-gal system; a split luciferase system; a split mCherry system; a split FRET system; and a split biotin system.

91. The composition of claim 88, wherein said antibody peptide comprises a bispecific antibody peptide or a fragment thereof.

92. The composition of claim 91, wherein said bispecific antibody peptide or a fragment thereof comprises a bispecific antibody peptide selected from the group consisting of: CD3; and EGFR.

93. The composition of claim 88, wherein said tumor specific antigen peptide comprises tumor specific antigen selected from the group consisting of: dopachrome-tautomerase (TRP2), melanocyte protein PMEL (gp100), HPV E6/7, MAGE 1, MAGE 3, NY-ESO, androgen receptor (AR), BCL-1, calprotectin, carcinoembryonic antigen (CEA), EGFRs, epithelial cell adhesion molecule (Ep-CAM), epithelial sialomucin, membrane estrogen receptors (mER), FAP HER2/neu, human high molecular weight melanoma-associated antigen (HMW-MAA), IL-6, MOC-1, MOC-21, MOC-52, melan-A/MART-1, melanoma-associated antigen, mucin, OKT9, progesterone receptor (PGR), prostate specific antigen (PSA), prostate stem cell antigen (PSCA), prostate-specific membrane antigen (PSMA), symaptophysin, VEGFRs, CD19, CD20, CD22, CD30 and CD33.

94. The composition of claim 88, wherein said immune response comprises CD8-T cell activation in a subject.

95. The composition of claim 88, and further comprising one or more proteins that disrupt macrophage or dendritic cell clearance of said EV by, or alternatively further comprising one or more proteins that promotes macrophage or dendritic cell clearance of said EV.

96. The composition of claim 95, and further comprising one or more proteins that disrupt macrophage or dendritic cell clearance of said EV by, or alternatively further comprising one or more proteins that promotes macrophage or dendritic cell clearance of said EV.

97. The composition of claim 88, and further comprising a protein configured to increase delivery of said target molecule comprises a protein or protein fragment selected from the group consisting of: a protein or protein fragment having a Gag peptide motif, and a p6^{Gag} peptide according to SEQ ID NO. 2.


SEQUENCE LISTING (12 sequences)

SEQ ID NO: 1 - protein, 511 aa, Vesicular Stomatitis Virus
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 52 aa, Human immunodeficiency virus type 1
LQSRPEPTAPPEESFRSGVETTTPPQKQEPIDKELYPLTSLRSLFGNDPSSQ

SEQ ID NO: 3 - DNA, 25 bp, Artificial
  PINK1, Forward
caccgcctggaggtgacaaagagca

SEQ ID NO: 4 - DNA, 25 bp, Artificial
  PINK1, Reverse
caccgcctggaggtgacaaagagca

SEQ ID NO: 5 - DNA, 18 bp, Artificial
  GAPDH Forward
gaaggtgaaggtcggagt

SEQ ID NO: 6 - DNA, 20 bp, Artificial
  GAPDH Reverse
gaagatggtgatgggatttc

SEQ ID NO: 7 - DNA, 18 bp, Artificial
  PINK1Ex1
cgctgctgctgcgcttca

SEQ ID NO: 8 - DNA, 20 bp, Artificial
  PINK1Ex3
ctgctccatactccccagcc

SEQ ID NO: 9 - DNA, 20 bp, Artificial
  PINK1Int2
gtctccataatcagacacct

SEQ ID NO: 10 - DNA, 20 bp, Artificial
  PINK1Int3
ggatggtgaactaaccaatc

SEQ ID NO: 11 - DNA, 22 bp, Artificial
  mPCSK9, Forward
gatgccactttacttcggagga

SEQ ID NO: 12 - DNA, 22 bp, Artificial
  mPCSK9, Reverse
aggaggattggagtggggatta